(19) United States
(12) Patent Application Publication
(10) Pub. No.: US 2012/0064569 A9
(43) Pub. Date: Mar. 15, 2012

(54) PRODUCTION OF ATTENUATED NEGATIVE STRANDED RNA VIRUS VACCINES FROM CLONED NUCLEOTIDE SEQUENCES

(76) Inventors: Brian R. Murphy, Bethesda, MD (US); Peter L. Collins, Rockville, MD (US); Anna P. Durbin, Takoma Park, MD (US); Mario H. Skiadopoulos, Potomac, MD (US)

(21) Appl. No.: 12/136,765

(22) Filed: Jun. 10, 2008

Related U.S. Application Data
(63) Continuation of application No. 10/916,827, filed on Aug. 11, 2004, now abandoned.
Application No. 10/916,827 is a continuation of application No. 09/958,292, filed on Jan. 8, 2002, now abandoned.
Application No. 09/958,292 is a continuation-in-part of application No. 09/887,469, filed on Jun. 22, 2001, now Pat. No. 6,923,971.
Application No. 09/887,469 is a continuation-in-part of application No. 09/847,173, filed on May 3, 2001, now Pat. No. 6,790,449.
Application No. 09/847,173 is a division of application No. 08/720,132, filed on Sep. 27, 1996, now Pat. No. 6,264,957.
Application No. 08/720,132 is a continuation-in-part of application No. 10/722,000, filed on Nov. 25, 2003, now Pat. No. 7,846,455.
Application No. 10/722,000 is a continuation of application No. 09/291,894, filed on Apr. 13, 1999, now Pat. No. 6,689,367.
Application No. 09/291,894 is a continuation-in-part of application No. 08/892,403, filed on Jul. 15, 1997, now Pat. No. 5,993,824.
(60) Provisional application No. 60/129,006, filed on Apr. 13, 1999.
(60) Provisional application No. 60/213,708, filed on Jun. 23, 2000.
(60) Provisional application No. 60/007,083, filed on Sep. 27, 1995.
(60) Provisional application No. 60/047,634, filed on May 23, 1997.
(60) Provisional application No. 60/046,141, filed on May 9, 1997.
(60) Provisional application No. 60/021,773, filed on Jul. 15, 1996.

(51) Int. Cl. C12P 21/00; C12N 15/63; C12N 7/01; C12N 15/44

(52) U.S. Cl. 435/69.1; 435/235.1; 536/23.72; 435/320.1

(57) ABSTRACT

Attenuated, recombinant negative stranded RNA viruses suitable for vaccine use are produced from one or more isolated polynucleotide molecules encoding the virus. A recombinant genome or antigenome of the subject virus is modified to encode a mutation within a recombinant protein of the virus at one or more amino acid positions(s) corresponding to a site of an attenuating mutation in a heretologous, mutant negative stranded RNA virus. A similar attenuating mutation as identified in the heterologous negative stranded RNA virus is thus incorporated at a corresponding site within the recombinant virus to confer an attenuated phenotype on the recombinant virus. The attenuating mutation incorporated in the recombinant virus may be identical or conservative in relation to the attenuating mutation identified in the heterologous, mutant virus. By the transfer of mutations into recombinant negative stranded RNA viruses in this matter, candidate vaccine viruses are engineered to elicit a desired immune response against a subject virus in a host susceptible to infection thereby.

### BACKGROUND OF THE INVENTION

Negative stranded RNA viruses comprise a diverse order (Mononegavirales) of important and highly destructive pathogens. Human pathogens within this group include rabies virus (RaV), measles virus (MeV), mumps virus (MuV), respiratory syncytial virus (RSV) and parainfluenza viruses (PIV) of several genotypes. In the case of RSV, this pathogen outranks all other microbial pathogens as a cause of pneumonia and bronchiolitis in infants under one year of age. RSV is responsible for more than one in five pediatric hospital admissions due to respiratory tract disease and causes an estimated 91,000 hospitalizations and 4,500 deaths yearly in the United States alone. Human PSV viruses (e.g., HPIV1, HPIV2 and HPIV3) also exact a heavy toll among human populations, causing bronchiolitis, croup and pneumonia primarily in infants and children. Karron et al., J. Infect. Dis. 172: 1445-50 (1995); Collins et al. “Parainfluenza Viruses”, p. 1205-1243. In B. N. Fields et al., eds., Fields Virology, 3rd ed, vol. 1. Lippincott-Raven Publ., Philadelphia (1996); Murphy et al., Virus Res. 11:1-15 (1988). Infections by human PIV viruses are responsible for approximately 20% of hospitalizations among young infants and children for respiratory tract infections.

Despite decades of investigation to develop effective vaccine agents against RSV and PIV, no safe and effective vaccines have yet been approved to prevent the severe morbidity and significant mortality associated with these viruses. Other important members of the Mononegavirales similarly await effective vaccine development or would benefit from improved vaccines. One obstacle to development of live vaccines against negative stranded RNA viruses is the difficulty in achieving an appropriate balance between attenuation and immunogenicity. Genetic stability of attenuated viruses also can be a problem. Vaccine development in the case of RSV is also impeded by the relatively poor growth of RSV in cell culture and the instability of the virus particle. Another feature of RSV infection is that the immunity which is induced is not fully protective against subsequent infection A number of factors probably contribute to this, including the relative inefficiency of the immune system in restricting virus infection on the luminal surface of the respiratory tract, the short-lived nature of local mucosal immunity, rapid and extensive virus replication, reduced immune responses in the young due to immunological immaturity, immunosuppression by transplacentally derived maternal serum antibodies, and certain features of the virus such as a high degree of glycosylation of the G protein. Also, as will be described below, RSV exists as two antigenic subgroups A and B, and immunity against one subgroup is of reduced effectiveness against the other.

Formalin-inactivated RSV has been tested as a vaccine against RSV in the mid-1960s, but failed to protect against RSV infection or disease, and in fact exacerbated symptoms during subsequent infection by the virus. (Kim et al., Am. J. Epidemiol., 89:422-434 (1969), Chin et al., Am. J. Epidemiol., 89:449-463 (1969); Kapikian et al., Am. J. Epidemiol., 89:405-421 (1969)). More recently, vaccine development for RSV has focused on attenuated RSV mutants. Friedewald et al., J. Amer. Med. Assoc. 204:690-694 (1968) reported a cold passaged mutant of RSV (cpRSV) which appeared to be sufficiently attenuated to be a candidate vaccine. This mutant exhibited a slight increased efficiency of growth at 26° C. compared to its wild-type (wt) parental virus, but its replication was neither temperature sensitive nor significantly cold-adapted. The cold-passaged mutant, however, was attenuated for adults. Although satisfactorily attenuated and immunogenic for infants and children who had been previously infected with RSV (i.e., seropositive individuals), the cpRSV mutant retained a low level virulence for the upper respiratory tract of seronegative infants.

Similarly, Gharpure et al., J. Virol. 3:414-421 (1969) reported the isolation of temperature sensitive RSV (tsRSV) mutants which are also promising vaccine candidates. One mutant, ts-1, was evaluated extensively in the laboratory and in volunteers. The mutant produced asymptomatic infection in adult volunteers and conferred resistance to challenge with wild-type virus 45 days after immunization. Again, while seropositive infants and children underwent asymptomatic infection, seronegative infants developed signs of rhinitis and other mild symptoms. Furthermore, instability of the ts phenotype was detected, although virus exhibiting a partial or complete loss of temperature sensitivity represented a small proportion of virus recoverable from vaccinees, and was not associated with signs of disease other than mild rhinitis.

These and other studies revealed that certain cold-passaged and temperature sensitive RSV strains were underattenuated and caused mild symptoms of disease in some vaccinees, particularly seronegative infants, while others were overattenuated and failed to replicate sufficiently to elicit a protective immune response, (Wright et al., Infect. Immun., 37:397-400 (1982)). Moreover, genetic instability of candidate vaccine mutants has resulted in loss of their temperature-sensitive phenotype, further hindering development of effective RSV vaccines. See generally, Hodes et al., Proc. Soc. Exp. Biol. Med. 145:1158-1164 (1974), McIntosh et al., Pediatr. Res. 8:689-696 (1974), and Belshe et al., J. Med. Virol., 3:101-110 (1978).

Abandoning the approach of creating suitably attenuated RSV strains through poorly controlled biological methods such as cold-passaging, investigators tested subunit vaccine candidates using purified RSV envelope glycoproteins. The glycoproteins induced resistance to RSV infection in the lungs of cotton rats, Walsh et al., J. Infect. Dis. 155:1198-1204 (1987), but the antibodies had very weak neutralizing activity and immunization of rodents with purified subunit vaccine led to disease potentiation reminiscent of the formerly treated RSV vaccine (Murphy et al., Vaccine 8:497-502 (1990)).

Vaccinia virus recombinant-based vaccines which express the F or G envelope glycoprotein have also been explored. These recombinants express RSV glycoproteins which are indistinguishable from the authentic viral counterpart, and rodents infected intradermally with vaccinia-RSV F and G recombinants developed high levels of specific antibodies that neutralized viral infectivity. Indeed, infection of cotton rats with vaccinia-F recombinants stimulated almost complete resistance to replication of RSV in the lower respiratory tract and significant resistance in the upper tract (Olmsted et al., Proc. Natl. Acad. Sci. USA 83:7462-7466 (1986)). However, immunization of chimpanzees with vaccinia-F and -G recombinant provided almost no protection against RSV challenge in the upper respiratory tract (Collins et al., Vaccine 8:164-168 (1990)) and inconsistent protection in the lower respiratory tract (Crowe et al., Vaccine 11:1395-1404 (1993)).

The unfulfilled promises of biologically attenuated viral strains, subunit vaccines, and other strategies for vaccine development to treat RSV and other negative stranded RNA viruses underscores a need for new methods to develop novel vaccines, particularly methods for manipulating recombinant vaccine candidates to incorporate genetic changes to yield new phenotypic properties in viable, attenuated recombinants. However, manipulation of the genomic RNA of RSV and other negative-sense RNA viruses has heretofore proven difficult. Major obstacles in this regard include non-infectivity of naked genomic RNA of these viruses, poor viral growth in tissue culture, lengthy replication cycles, virion instability, a complex genome, and a refractory organization of gene products.

In the case of PIV3, two live attenuated vaccine candidates have received particular attention. One of these candidates is a bovine PIV3 (BPIV3) strain that is antigenically related to HPIV3, and which has been shown to protect animals against HPIV3. BPIV3 is attenuated, genetically stable and immunogenic in human infants and children (Karron et al., J. Inf. Dis. 171:1107-14 (1995a); Karron et al., J. Inf. Dis. 172:1445-1450, (1995b)). A second PIV3 vaccine candidate, JS cp45 is a cold-adapted mutant of the JS wildtype (wt) strain of HPIV3 (Karron et al., (1995b), supra; Belshe et al., J. Med. Virol. 10:235-42 (1982)). This live-attenuated, cold-passaged (cp) PIV3 vaccine candidate exhibits temperature-sensitive (ts), cold-adaptation (ca), and attenuation (att) phenotypes which are stable after viral replication in vivo. The cp45 virus is protective against human PIV3 challenge in experimental animals and is attenuated, genetically stable, and immunogenic in seronegative human infants and children (Hall et al., Virus Res. 22:173-184 (1992); Karron et al., J. Infect. Dis. 172(6): 1445-1450 (1995b), supra).

Recombinant DNA technology has made it possible to recover infectious negative-stranded RNA viruses from cDNA, to genetically manipulate viral clones to construct novel vaccine candidates, and to rapidly evaluate their level of attenuation and phenotypic stability (for reviews, see Conzelmann, J. Gen. Virol. 77:381-89 (1996); Palese et al., Proc. Natl. Acad. Sci. U.S.A. 93:11354-58, (1996)). In this context, recombinant rescue has been reported for infectious respiratory syncytial virus (RSV), human parainfluenza virus 3 (HPIV3), rabies virus (RaV), vesicular stomatitis virus (VSV), measles virus (MeV), Sendai virus (SeV) rinderpest virus, simian virus type S, and bovine RSV from cDNA-encoded antigenomic RNA in the presence of essential viral proteins (see, e.g., Garcin et al., EMBO J. 14:6087-6094 (1995); Lawson et al., Proc. Natl. Acad. Sci. U.S.A. 92:4477-81 (1995); Radecke et al., EMBO J. 14:5773-5784 (1995); Schnell et al., EMBO J. 13:4195-203 (1994); Whelan et al., Proc. Natl. Acad. Sci. U.S.A. 92:8388-92 (1995); Hoffman et al., J. Virol. 71:4272-4277 (1997); Kato et al., Genes to Cells 1:569-579 (1996), Roberts et al., Virology 247(1), 1-6 (1998); Baron et al., J. Virol. 71:1265-1271 (1997); International Publication No. WO 97/06270; Collins et al., Proc. Natl. Acad. Sci. USA 92:11563-11567 (1995); Durbin et al., Virology 235:323-332 (1997); U.S. patent application Ser. No. 08/892,403, filed Jul. 15, 1997 (corresponding to published International Application No. WO 98/02530 and priority U.S. Provisional Application Nos. 60/047,634, filed May 23, 1997, 60/046,141, filed May 9, 1997, and 60/021,773, filed Jul. 15, 1996); Juhasz et al., J. Virol. 71(8):5814-5819 (1997); He et al. Virology 237:249-260 (1997); Whitehead et al., Virology 247(2):232-9 (1998a); Whitehead et al., J. Virol. 72(5):4467-4471 (1998b); Jin et al. Virology 251:206-214 (1998); Bucholz et al. J. Virol. 73:251-259 (1999); and Whitehead et al., J. Virol. 73:3438-3442 (1999), each incorporated herein by reference).

Despite the availability of reverse genetics methods to recover and modify recombinant, negative stranded RNA viruses, an urgent need remains in the art for additional tools and methods to engineer safe and effective vaccines to alleviate the serious health problems attributable to RSV, PIV and other pathogens within the Mononegavirales. Among the remaining challenges in this context is the difficulty of achieving suitably attenuated, immunogenic and genetically stable vaccine candidates. To achieve this goal, existing methods for identifying and incorporating attenuating mutations into recombinant viral strains must be expanded. Surprisingly, the present invention fulfills this goal and provides additional advantages as described hereinbelow.

### SUMMARY OF THE INVENTION

The present invention provides novel methods and compositions for designing and producing attenuated, recombinant negative stranded RNA viruses suitable for vaccine use. According to the methods of the invention, recombinant negative stranded RNA viruses are produced from one or more isolated polynucleotide molecules encoding the virus. This is achieved by coexpressing in a cell or cell-free system one or more polynucleotide molecules encoding a recombinant genome or antigenome of the virus along with essential viral proteins necessary to produce an infectious virus or viral particle.

The recombinant genome or antigenome of the subject virus is modified to encode a mutation within a recombinant protein of the virus at one or more amino acid position(s) corresponding to a site of an attenuating mutation in a heterologous, mutant negative stranded RNA virus. The mutation which is thus “transferred” in this adoptive or iterative fashion surprisingly confers an attenuated phenotype on the recombinant virus. In this manner, candidate vaccine viruses are recombinantly engineered to elicit an immune response against selected negative stranded RNA viruses in a host susceptible to infection by the subject virus.

In related aspects of the invention, isolated polynucleotide molecules and vectors are provided that encode an attenuated, recombinant negative stranded viral genome or antigenome. Consistent with the above aspects, the subject genome or antigenome encodes a mutation within a selected, recombinant protein of the virus at an amino acid position(s) corresponding to the site of an attenuating mutation in a heterologous, mutant negative stranded RNA virus.

Also provided within the invention are methods and compositions incorporating attenuated, recombinant negative stranded RNA virus mutated as above for prophylaxis and treatment of infection by the subject virus.

In preferred embodiments of the invention, the recombinant negative stranded RNA virus is either a respiratory syncytial virus (RSV), parainfluenza virus (PIV) or measles virus. In conjunction with each of these embodiments, the heterologous, mutant negative stranded RNA virus may be a heterologous RSV, human parainfluenza virus (HPIV1, HPIV2, HPIV3), bovine PIV (BPIV), Sendai virus (SeV), Newcastle disease virus (NDV), simian virus 5 (SV5), Mumps virus (MuV), measles virus (MeV), canine distemper virus (CDV), rabies virus (RaV), or vesicular stomatitis virus (VSV).

Various target proteins are amenable to introduction of attenuating mutations from one negative stranded RNA virus at a corresponding site within a heterologous protein, in accordance with the methods of the invention. Throughout the order Mononegavirales, five target proteins are strictly conserved and show moderate to high degrees of sequence identity for specific regions or domains. In particular, all known members of the order share a homologous constellation of five proteins: a nucleocapsid protein (N), a nucleocapsid phosphoprotein (P), a nonglycosylated matrix (M) protein, at least one surface glycoprotein (HN, H, or G) and a large polymerase (L) protein. These proteins all represent useful targets for incorporating attenuating mutations by altering one or more conserved residues in a protein of the recombinant virus at a site corresponding to the site of an attenuating mutation identified in the heterologous, mutant virus.

In more detailed aspects of the invention, additional proteins are targeted that may be shared only among particular families, subfamilies, genera or species within the Mononegavirales. For example, all members of the Paramyxovirus family have at least two surface glycoproteins, HN (or H or G) and F. Almost all members of the Respirovirus, Rubulavirus and Morbillivirus genera have a cysteine-rich protein V. Respiroviruses and Morbilliviruses also encode homologous C proteins. Pneumoviruses and a subset of Rubulaviruses (Simian virus 5 (SV5) and mumps virus (MuV)) share homologous surface glycoproteins SH. Within the Pneumovirus genus (including bovine, ovine and caprine RSV and pneumonia virus of mice—alternatively referred to herein as murine RSV), several additional proteins, NS1, NS2, M2(ORF1) and M2(ORF2) are conserved. Avian pneumoviruses lack NS1 and NS2 but have M2(ORF1), M2(ORF2), and SH proteins. Each of the foregoing proteins provide useful targets for heterologous transfer of attenuating mutations between taxa sharing the target protein of interest.

In this context, the methods of the invention are based on identification of an attenuating mutation in a first negative stranded RNA virus. The mutation, identified in terms of mutant versus wild-type sequence at the subject amino acid position(s) marking the site of the mutation, provides an index for sequence comparison against a homologous protein in a different virus that is the target virus for recombinant attenuation. The attenuating mutation may be previously known or may be identified by mutagenic and reverse genetics techniques applied to generate and characterize biologically-derived mutant virus. Alternatively, attenuating mutations of interest may be generated and characterized de novo, eg., by site directed mutagenesis and conventional screening methods.

Each attenuating mutation identified in a negative stranded RNA virus provides an index for sequence comparison against a homologous protein in one or more heterologous negative stranded virus(es). In this context, existing sequence alignments may be analyzed, or conventional sequence alignment methods may be employed to yield sequence comparisons for analysis, to identify corresponding protein regions and amino acid positions between the protein bearing the attenuating mutation and a homologous protein of a different virus that is the target recombinant virus for attenuation. Where one or more residues marking the attenuating mutation have been altered from a “wild-type” identity that is conserved at the corresponding amino acid position(s) in the target virus protein, the genome or antigenome of the target virus is recombinantly modified to encode an amino acid deletion, substitution, or insertion to alter the conserved residue(s) in the target virus protein and thereby confer an analogous, attenuated phenotype on the recombinant virus.

Within this rational design method for constructing attenuated recombinant negative stranded viruses, the wild-type identity of residue(s) at amino acid positions marking an attenuating mutation in one negative stranded RNA virus may be conserved strictly, or by conservative substitution, at the corresponding amino acid position(s) in the target virus protein. Thus, the corresponding residue(s) in the target virus protein may be identical, or may be conservatively related in terms of amino acid side-group structure and function, to the wild-type residue(s) found to be altered by the attenuating mutation in the heterologous, mutant virus. In either case, analogous attenuation in the recombinant virus may be achieved according to the methods of the invention by modifying the recombinant genome or antigenome of the target virus to encode the amino acid deletion, substitution, or insertion to alter the conserved residue(s). In this context, it is preferable to modify the genome or antigenome to encode an alteration of the conserved residue(s) that corresponds conservatively to the alteration marking the attenuating mutation in the heterologous, mutant virus. For example, if an amino acid substitution marks a site of mutation in the mutant virus compared to the corresponding wild-type sequence, then a substitution should be engineered at the corresponding residue(s) in the recombinant virus. Preferably the substitution will be identical or conservative to the substitute residue present in the mutant viral protein. However, it is also possible to alter the native amino acid residue at the site of mutation non-conservatively with respect to the substitute residue in the mutant protein (e.g., by using any other amino acid to disrupt or impair the identity and function of the wild-type residue). In the case of mutations marked by deletions or insertions, these can transferred as corresponding deletions or insertions into the recombinant virus, however the particular size and amino acid sequence of the deleted or inserted protein fragment can vary.

Within alternative aspects of the invention, mutations incorporated within recombinant negative stranded virus may confer a variety of phenotypes in addition to or associated with the desired, attenuated phenotype. Thus, exemplary mutations incorporated within recombinant proteins of the virus may confer temperature sensitive (ts), cold-adapted (ca), small plaque (sp), or host range restricted (hr) phenotypes, or a change in growth or immunogenicity, in addition to or associated with the attenuated phenotype.

In exemplary embodiments of the invention, a ts or non-ts mutation is incorporated within the large polymerase L protein of a negative stranded virus, for example HPIV3. An attenuating mutation is identified in a heterologous, mutant negative stranded RNA virus (e.g. RSV), which may be any virus sharing a conservative protein structural relationship with PIV3 spanning the mutation of interest. In more detailed embodiments, the attenuating mutation in the heterologous, mutant virus comprises an amino acid substitution of phenylalanine at position 521 of the L protein of human RSV cpts530 (ATCC VR 2452).

In other exemplary embodiments, a ts or non-ts attenuating mutation is incorporated within a recombinant protein of a parainfluenza virus (PIV), for example, human PIV1 (HPIV1), human PIV2 (HPIV2), human PIV3 (HPIV3), bovine PIV (BPIV), or murine PIV (MPIV or Sendai virus). The recombinant genome or antigenome is modified to encode an amino acid substitution, deletion or insertion within a N, P, C, D, V, M, F, HN or L protein of the recombinant virus. The mutation may confer a ts or non-ts attenuation phenotype on the recombinant virus. In preferred aspects, the attenuating mutation in the heterologous, mutant negative stranded RNA virus corresponds to a mutation of the biologically-derived mutant HPIV3 strain JS cp45, and is preferably an amino acid substitution within the HPIV3 JS cp45 L protein. Exemplary mutations in this context include an amino acid substitution of tyrosine at position 942 of the L protein of HPIV3 JS cp45, an amino acid substitution of leucine at position 992 of the L protein of HPIV3 JS cp45, and/or an amino acid substitution of threonine at position 1558 of the L protein of HPIV3 JS cp45.

Yet additional mutations identified in heterologous, mutant negative stranded RNA virus for use in constructing recombinant PIV of the invention comprise an amino acid substitution within the F protein of HPIV3 JS cp45. In one example, the attenuating mutation in the heterologous virus comprises an amino acid substitution of isoleucine at position 420 of the F protein of HPIV3 JS cp45. Alternatively, the attenuating mutation may comprise an amino acid substitution of alanine at position 450 of the F protein of HPIV3 JS cp45. Likewise, attenuation of recombinant PIV can be achieved by modifying the recombinant PIV genome or antigenome to encode an analogous mutation to an attenuating mutation identified in RSV. In one such example described below, the attenuating mutation identified in RSV comprises an amino acid substitution of phenylalanine at position 521 of the RSV L protein. The PIV genome or antigenome is modified to encode an alteration of a conserved residue that corresponds conservatively to the alteration marking the attenuating mutation in the heterologous RSV mutant. In one embodiment, the mutation is incorporated within a recombinant HPIV3 protein and comprises an amino acid substitution of phenylalanine at a corresponding position 456 of the L protein of said HPIV3.

Yet additional PIV vaccine candidates within the invention can be achieved by modifying the recombinant PIV genome or antigenome to encode an analogous mutation to an attenuating mutation identified in Sendai virus (SeV). In one example described below, the attenuating mutation comprises an amino acid substitution of phenylalanine at position 170 of the C protein of SeV. The PIV genome or antigenome is modified to encode an alteration of a conserved residue that corresponds conservatively to the alteration marking the attenuating mutation in the heterologous, SeV mutant. In one embodiment, the mutation is incorporated within a recombinant HPIV3 protein and comprises an amino acid substitution of phenylalanine at position 164 of the C protein of HPIV3.

In additional exemplary embodiments, a ts or non-ts attenuating mutation is incorporated within a recombinant protein of a measles virus (MeV). The heterologous, mutant negative stranded RNA virus in which the attenuating mutation is identified may be a respiratory syncytial virus (RSV), human parainfluenza virus (HPIV)1, HPIV2, HPIV3, bovine PIV (BPIV), Sendai virus (SeV), Newcastle disease virus (NDV), simian virus 5 (SV5), Mumps virus (MuV), measles virus (MeV), canine distemper virus (CDV), rabies virus (RaV), or vesicular stomatitis virus (VSV). Preferably, the heterologous, mutant virus is HPIV3 JS cp45. In more detailed embodiments, the attenuating mutation identified in HPIV3 JS cp45 comprises an amino acid substitution of tyrosine at position 942 or leucine at position 992 of the L protein.

Yet additional embodiments of the invention are provided in which the recombinant negative stranded RNA virus is a chimeric virus. In particular, the virus has a recombinant genome or antigenome comprising a partial or complete genome or antigenome of one species, subgroup, or strain of negative stranded RNA virus combined with a heterologous gene or gene segment of a different species, subgroup, or strain of negative stranded RNA virus. The attenuating mutation may optionally be incorporated within a protein or protein region encoded by the heterologous gene or gene segment. In preferred aspects, the chimeric virus is a PIV having a complete genome or antigenome of one PIV species, subgroup, or strain combined with at least one gene or gene segment of the HN and F glycoprotein genes of a heterologous PIV species, subgroup, or strain. In other embodiments, the chimeric virus is a RSV in which the recombinant genome or antigenome incorporates a gene or gene segment of a F, G or SH glycoprotein gene from a heterologous RSV species, subgroup, or strain. Preferably, the F and G glycoprotein genes of human RSV subgroup A are substituted by the F and G glycoprotein genes of human RSV subgroup B in a RSV A background.

In other aspects of the invention, the recombinant negative stranded RNA virus is further modified or attenuated by additional changes to the recombinant genome or antigenome. In one embodiment, the genome or antigenome is further modified to encode one or more additional attenuating mutations adopted from a biologically-derived mutant negative strand RNA virus. For example in a recombinant RSV, the genome or antigenome is modified to encode at least one and up to a full complement of attenuating mutations present within a panel of biologically-derived mutant RSV strains, said panel comprising (ATCC VR 2450), cpts RSV 248/404 (ATCC VR 2454), cpts RSV 248/955 (ATCC VR 2453), cpts RSV 530 (ATCC VR 2452), cpts RSV 530/1009 (ATCC VR 2451), cpts RSV 530/1030 (ATCC VR 2455), RSV B-1 cp52/2B5 (ATCC VR 2542), and RSV B-1 cp-23 (ATCC VR 2579). Alternatively, in a recombinant PIV the recombinant genome or antigenome encodes at least one and up to a full complement of attenuating mutations present within HPIV3 JS cp45. Preferably, at least one of the attenuating mutation(s) contemplated herein are stabilized by multiple nucleotide changes in a codon specifying the mutation.

In other aspects of the invention, the recombinant negative stranded RNA virus is further modified or attenuated by a nucleotide modification specifying a phenotypic change selected from a change in growth characteristics, attenuation, temperature-sensitivity, cold-adaptation, small plaque size, host range restriction, or a change in immunogenicity. For example, in RSV the recombinant genome or antigenome may incorporate a modification of the SH, NS1, NS2 or G gene, such as a gene deletion or ablation of gene expression. Other nucleotide modifications in RSV and other negative stranded RNA viruses comprise a nucleotide deletion, insertion, addition or rearrangement within a cis-acting regulatory sequence or within the recombinant genome or antigenome.

In other related aspects the invention provides isolated, recombinant negative stranded RNA virus that are attenuated and elicit an immune response in a host susceptible to infection by the subject virus. The virus comprises a recombinant genome or antigenome and viral proteins necessary to produce an infectious viral particle of the RNA virus. The recombinant genome or antigenome is modified to encode a mutation within a recombinant protein of the virus at an amino acid position corresponding to an amino acid position of an attenuating mutation identified in a heterologous, mutant negative stranded RNA virus. The mutation, by incorporation within the recombinant protein, confers an attenuated phenotype on the recombinant virus.

Also provided are isolated polynucleotide molecules encoding a recombinant genome or antigenome of a recombinant negative stranded RNA virus. The recombinant genome or antigenome is likewise modified to encode a mutation within a recombinant protein of the virus at an amino acid position corresponding to an amino acid position of an attenuating mutation identified in a heterologous, mutant negative stranded RNA virus. The mutation, by incorporation within the recombinant protein, confers an attenuated phenotype on the recombinant virus. In related aspects, expression vectors are provided which comprise an operably linked transcriptional promoter, a polynucleotide molecule encoding a recombinant genome or antigenome of a recombinant negative stranded RNA virus as set forth above, and a transcriptional terminator.

The invention also provides a method for stimulating the immune system of an individual to induce protection against a negative stranded RNA virus. The method includes administering to the individual an immunologically sufficient amount of an attenuated, recombinant negative stranded virus as described above combined with a physiologically acceptable carrier. In related aspects, an immunogenic composition is provided which elicits an immune response against a negative stranded RNA virus. The composition comprises an immunologically sufficient amount of an attenuated, recombinant negative stranded virus of the invention combined with a physiologically acceptable carrier. Preferably, the attenuated, recombinant negative stranded virus is a RSV, PIV or measles virus.

### BRIEF DESCRIPTION OF THE DRAWINGS

FIG. 1, panel A provides an amino acid sequence alignment of the L polymerase proteins of RSV (SEQ ID NO. 44), PIV3 (SEQ ID NO. 45), measles (SEQ ID NO. 46) and Sendai (SEQ ID NO. 47) viruses spanning RSV Phe-521 (denoted by arrow). A consensus sequence (SEQ ID NO. 48) was generated from an exact match of at least three residues of each position. The numerical position of the first amino acid in the sequence presented is indicated. Residues conserved in all four viruses are in bold type. Underlined residues are also conserved in the PIV2, canine distemper virus, simian parainfluenza virus 41, simian parainfluenza virus 5, avian pneumovirus, Newcastle disease virus, Hendra virus, and rinderpest virus L polymerase proteins (Accession numbers P26676, P24658, P35341, Q88434, Y09630, U65312, X05399, AF017149, P41357, respectively). The position of the phenylalanine 521 to leucine mutation in RSV cpts530 corresponds to aa 456 in the PIV3 L polymerase.

FIG. 1, panel B provides a schematic representation of the F456L mutation and cp45 mutations that were introduced into PIV3 rwt. Relative positions of the introduced cp45 mutations=(*) and the F456L mutation=(♦).

FIG. 1, panel C shows the nucleotide sequence (positive sense) encoding the F456L mutation (SEQ ID NO. 50) compared to wt sequence (SEQ ID NO. 49). The PIV3 nucleotide sequence numbered according to the complete rwt antigenome is shown for the wt virus and the mutant sequence is shown below. Nucleotide changes are underlined and the codon bearing the F456L mutation is in bold type. The introduced Xmnl restriction endonuclease recognition site in the mutant sequence is in italic type.

FIG. 2 depicts replication of rcp45 and rcp45-456 in the upper respiratory tract of chimpanzees. Mean virus titers in the nasopharyngeal swab specimens on the indicated date post-infection from animals infected with □, rcp45-456, n=6, or ▪, rcp45 n=4. Statistically significant difference between indicated values: (a) P<0.005; (b) p<0.05; (c) p<0.025; Student's t-test. (d) Limit of detection ≦0.5 log₁₀ TCID₅₀/ml.

FIG. 3 depicts organization of the HPIV3 P/C/D/V ORFs (not to scale). The three reading frames of the P mRNA are shown (+1, +2 and +3) with the P, C, D and V ORFs represented by rectangles. Amino acid lengths are indicated. The position of the RNA editing site is shown as a vertical line and its sequence motif is shown and numbered according to its nucleotide position in the complete HPIV3 antigenomic sequence.

FIG. 3, panel A depicts organization of the unedited P mRNA. The sequence containing the translational start sites of the P and C ORFs is shown (SEQ ID NO. 52) and is numbered according to the complete antigenomic sequence. The nucleotide positions of the P, C, D, and V ORFs in the complete antigenomic sequence are: P, 1784-3595; C, 1794-2393; D, 2442-2903; V, 2792-3066. Relative to the P mRNA, the AUG that opens the P ORF is at positions 80-82.

FIG. 3, panel B depicts organization of an edited version of the P mRNA that contains an insertion of two nontemplated G residues (GG) (SEQ ID NO. 53) in the editing site (SEQ ID NO. 51). This changes the reading register so that the upstream end of the P ORF in frame+2 is fused to the D ORF in frame+3. The resulting chimeric protein contains the N-terminal 241 amino acids encoded by the P ORF fused to the C-terminal 131 amino acids encoded by the D ORF.

FIG. 4 shows results of a radioimmunoprecipitation assay demonstrating expression of the C protein in rF164S. Lane (a) ³⁵S-labeled cell lysates were immunoprecipitated with a polyclonal C-specific rabbit antiserum. A 22 kD band corresponding to the C protein (open arrow) is clearly present in rJS and rF164S lysates. Lane (b) cell lysates were immunoprecipitated by a mixture of two monoclonal antibodies specific to the HPIV3 HN protein. The 64 kD band corresponding to the HN protein (closed arrow) is present in each virus lysate confirming they are indeed HPIV3 and express similar levels of proteins. Mock lane indicates tissue culture lysates harvested from uninfected cells.

FIG. 5 shows the results of multicycle replication of the recombinant mutant virus rF164S compared with the parent virus rJS. The virus titers are shown as TCID₅₀/ml and are the average of duplicate samples.

### DESCRIPTION OF THE SPECIFIC EMBODIMENTS

The methods of the invention provide attenuated, recombinant negative stranded RNA viruses suitable for vaccine use. The recombinant negative stranded RNA viruses are produced from one or more isolated polynucleotide molecules encoding the virus. Production of a recombinant virus is achieved by coexpressing in a cell or cell-free system one or more polynucleotide molecules that encode: (i) a recombinant genome or antigenome of the virus and (ii) essential viral proteins necessary to produce an infectious virus or subviral particle.

The recombinant genome or antigenome of the subject recombinant virus is modified to encode a mutation within a recombinant protein of the virus at one or more amino acid position(s) corresponding to a site of an attenuating mutation in a heterologous, mutant negative stranded RNA virus. The attenuating mutation identified in the heterologous negative stranded RNA virus is thus “transferred” to a corresponding site within the recombinant virus to confer an attenuated phenotype on the recombinant virus. The transferred mutation typically is identical or conservative to the attenuating mutation identified in the heterologous, mutant virus, although non-conservative substitutions also can be made. By incorporation of transferred mutations into recombinant negative stranded RNA viruses in this manner, candidate vaccine viruses are engineered to elicit a desired immune response against a subject virus in a host susceptible to infection thereby.

The instant invention embodies a novel paradigm for rationally designing attenuated vaccine viruses based on the identification of attenuating mutations in a heterologous negative stranded RNA virus. Attenuating mutations in the heterologous virus are mapped to one or more amino acid deletion(s), substitution(s), or insertion(s) in a protein of interest in the heterologous, mutant virus, eg., by conventional nucleotide or amino acid sequence comparison between the mutant virus and its non-attenuated parental virus. In this context, the parental virus is typically a biologically-derived strain that is wild-type, at least for the attenuated phenotype. However, partially attenuated mutant strains may also be used, wherein an additional attenuating mutation may arise through artificial mutagenesis or by natural polymorphism, etc. In addition, parental virus in which attenuating mutations may be introduced and subsequently mapped include artificially produced virus such as are provided through cDNA viral clones in accordance with known reverse genetic methods.

Thus, the attenuating mutations identified in heterologous negative stranded RNA viruses may be previously known or may be generated and/or identified by conventional mutagenic and/or reverse genetics techniques. These techniques may be applied to generate and characterize biologically-derived mutant virus, or to generate and characterize attenuating mutations of interest de novo, eg., by site directed mutagenesis of a wild-type or non-attenuated mutant viral cDNA clone in conjunction with conventional screening methods to identify attenuated derivatives. Reverse genetic methods for recovery and genetic manipulation of infections viral clones are known for representative viral groups throughout the Mononegavirales (for reviews, see Conzelmann, J. Gen. Virol. 77:381-89 (1996); Palese et al., Proc. Natl. Acad. Sci. U.S.A. 93:11354-58, (1996)).

For example, rescue of infectious viral clones has been reported for rabies virus (RaV), vesicular stomatitis virus (VSV), measles virus (MeV), and Sendai virus (SeV) rinderpest (Baron et al., J. Virol. 71:1265-1271 (1997)) and simian virus S (He et al., Virology 237:249-260 (1997), see page 5) from cDNA-encoded antigenomic RNA coexpressed with essential viral proteins for infectivity, namely the nucleocapsid N, phosphoprotein P, and large polymerase subunit L (see, eg., Garcin et al., EMBO J. 14:6087-6094 (1995); Lawson et al., Proc. Natl. Acad. Sci. U.S.A. 92:4477-81 (1995): Radecke et al., EMBO J. 14:5773-5784 (1995); Schnell et al., EMBO J. 13:4195-203 (1994); Whelan et al., Proc. Natl. Acad. Sci. U.S.A. 92:8388-92 (1995); and International Publication No. WO 97/06270, each incorporated herein by reference).

Rescue of infectious respiratory syncytial virus (RSV) has also been achieved through development of a novel system using cDNA-encoded antigenomic RNA coexpressed with the nucleocapsid N, phosphoprotein P, large polymerase subunit L, and a previously uncharacterized product of the M2 ORF1 gene (see, U.S. patent application Ser. No. 08/720,132, filed Sep. 27, 1996, which is a continuation of U.S. Provisional Application No. 60/007,083, filed Sep. 27, 1995, and U.S. patent application Ser. No. 08/892,403, filed Jul. 15, 1997, which corresponds to published International Application No. WO 98/02530 and is a continuation-in-part of U.S. Provisional Application Nos. 60/047,634, filed May 23, 1997, 60/046,141, filed May 9, 1997, and 60/021,773, filed Jul. 15, 1996, each incorporated herein by reference). These disclosures include description of representative constructs for use in producing infectious, recombinant RSV, including recombinant RSV clones incorporating attenuating mutations adopted from biologically-derived RSV mutants. One such construct is a recombinant viral clone incorporating an attenuating mutation of the RSV mutant cpts530, designated D53-530-sites ( ). Further description of methods and compositions for recovery and recombinant manipulation of RSV clones is provided in Collins et al., Proc. Natl. Acad. Sci. USA 92:11563-7 (1995); Juhasz et al., Vaccine 17:1416-1424 (1999); Juhasz et al., J. Virol. 71(8):5814-5819 (1997); Whitehead et al., Virology 247(2):232-9 (1998a); Whitehead et al., J. Virol. 72(5):4467-4471 (1998b); and Whitehead et al., J. Virol. 73:(4)3438-3442 (1999), each incorporated herein by reference.

In another important example, rescue of infectious parainfluenza virus (PIV) has also been achieved through using cDNA-encoded antigenomic RNA coexpressed with the N, P and L proteins (see, U.S. patent application Ser. No. 09/083,793 filed May 22, 1998, which corresponds to published International Application No. WO 98/53078 and is a continuation-in-part of U.S. Provisional Application Nos. 60/047,575 filed May 23, 1997, and 60/059,385, each incorporated herein by reference). These references include description of the following plasmids for use in producing infectious PIV clones: p3/7(131) (ATCC 97990); p3/7(131)2G (ATCC 97989); and p218(131) (ATCC 97991); each deposited under the terms of the Budapest Treaty with the American Type Culture Collection (ATCC) of 10801 University Boulevard, Manassas, Va. 20110-2209.

Development of reverse genetics systems for recovery and manipulation of negative stranded RNA viruses allows for detailed analysis and mapping of attenuating mutations to develop useful recombinant vaccine candidates specific to the subject viral species. Heretofore, transfer of attenuating mutations by recombinant methods within the Order Mononegarirales has not been tested or achieved. Nonetheless, the wide range of attenuated mutant strains identified for such representative taxa as RSVs, PIVs, measles and other members of the Mononegavirales, combined with the powerful tools provided through reverse genetics, serves as a rich source for determining useful mutations for heterologous transfer within the methods of the invention.

Attenuating mutations in heterologous negative stranded RNA viruses may be identified in biologically-derived mutant strains for incorporation within a heterologous, recombinant virus of the invention. The subject mutations may occur naturally or may be introduced into wild-type or partially attenuated parental strains by well known mutagenesis procedures. For example, attenuated mutant viral strains can be produced by chemical mutagenesis during virus growth in cell cultures to which a chemical mutagen has been added, by selection of virus that has been subjected to passage at suboptimal temperatures in order to introduce growth restriction mutations, or by selection of a mutagenized virus that produces small plaques (sp) or temperature sensitive (ts) virus in cell culture (see, eg., U.S. patent application Ser. No. 08/327,263, incorporated herein by reference).

By “biologically-derived” mutant is meant any mutant virus that is not produced by recombinant means. Thus, biologically-derived mutants include naturally occurring mutants having genomic variations from a reference wild-type sequence, eg., partially attenuated mutant PIV strains. Likewise, biologically-derived mutants include mutants derived from any parental viral strain without recombinant methods by, inter alia, artificial mutagenesis and selection procedures.

One well known procedure for generating biologically-derived negative stranded RNA virus involves subjecting a wild type or partially attenuated virus to passage in cell culture at progressively lower, attenuating temperatures. For example in the case of RSV, wild-type virus is typically cultivated at approximately 34-37° C. Partially attenuated mutants are produced by passage in cell cultures (e.g., primary bovine kidney cells) at suboptimal temperatures, e.g., 20-26° C. Thus, the cp mutant or other partially attenuated strain, eg., ts-1 or spRSV, is adapted to efficient growth at a lower temperature by passage in MRC-5 or Vero cells, down to a temperature of about 20-24° C. preferably 20-22° C. This selection of mutant RSV during cold-passage substantially eliminates any residual virulence in the derivative strains as compared to the partially attenuated parent.

Alternatively, specific mutations can be introduced into biologically-derived viruses by subjecting a wild type or partially attenuated parent virus to chemical mutagenesis, eg., to introduce ts mutations or, in the case of viruses which are already ts, additional ts mutations sufficient to confer increased attenuation and/or stability of the ts phenotype on the attenuated derivative. Means for the introduction of ts mutations into negative stranded RNA viruses include replication of the virus in the presence of a mutagen such as 5-fluorouridine or 5-fluorouracil in a concentration of about 10⁻³ to 10⁻⁵ M, preferably about 10⁻⁴ M, exposure of virus to nitrosoguanidine at a concentration of about 100 μg/ml, according to the general procedure described in, e.g., Gharpure et al., J. Virol. 3:414-421 (1969) and Richardson et al., J. Med. Virol. 3:91-100 (1978). Other chemical mutagens can also be used. Attenuation can result from a ts mutation in almost any viral gene, although a particularly amenable target for this purpose has been found to be the highly conserved polymerase (L) gene.

The level of temperature sensitivity of replication in exemplary attenuated virus for use within the invention is determined by comparing its replication at a permissive temperature with that at several restrictive temperatures. The lowest temperature at which the replication of the virus is reduced 100-fold or more in comparison with its replication at the permissive temperature is termed the shutoff temperature. In experimental animals and humans, both the replication and virulence of exemplary mutant RSV strains approximately correlate with the mutant's shutoff temperature. Replication of mutants with a shutoff temperature of 39° C. is moderately restricted, whereas mutants with a shutoff of 38° C. replicate less well and symptoms of illness are mainly restricted to the upper respiratory tract. A virus with a shutoff temperature of 35 to 37° C. will typically be fully attenuated in humans. Thus, attenuated biologically-derived mutant RSV for use within the invention which are ts will have a shutoff temperature in the range of about 35 to 39° C., and preferably from 35 to 38° C. The addition of a ts mutation into a partially attenuated strain produces multiply attenuated virus useful within vaccine compositions of the invention.

A number of attenuated RSV strains as candidate vaccines have been developed using multiple rounds of chemical mutagenesis to introduce multiple mutations into a virus which had already been attenuated during cold-passage (e.g., Connors et al., Virology 208: 478-484 (1995); Crowe et al., Vaccine 12: 691-699 (1994a); and Crowe et al., Vaccine 12: 783-790 (1994b), incorporated herein by reference). Evaluation of these biologically-derived mutants in accepted rodent and chimpanzee models, as well as in human adults and infants, indicates that certain of these candidate vaccine strains are genetically stable, highly immunogenic, and attenuated. Similar descriptions of attenuated mutant viruses are provided for PIV and other negative stranded RNA viral subjects of the invention (see, eg., U.S. patent application Ser. No. 08/892,403 and corresponding International Application No. WO 98/02530; U.S. patent application Ser. No. 09/083,793 and corresponding International Application No. WO 98/53078, each incorporated herein by reference).

In accordance with the methods of the invention, nucleotide sequence analysis of attenuated mutant viruses involving comparison of mutant to parental, eg., wild-type or partially attenuating, DNA or amino acid sequences can be employed to map attenuating mutations to specific nucleotide and amino acid changes. Often these changes will involve individual amino acid substitutions, however other mutations subject to conservative transfer according to the methods of the invention involve multiple amino acid substitutions, amino acid insertions or deletions, as well as more extensive alterations of conserved regions or domains within a target protein.

By employing the above-noted reverse genetic methods, nucleotide and amino acid changes identified in an attenuated mutant virus can be introduced into a previously characterized, cDNA-encoded virus, thereby allowing the artisan to distinguish between silent, incidental mutations and those responsible for the desired phenotypic changes. In this regard, subject mutations are introduced separately and in various combinations into the genome or antigenome of infectious RSV clones. This process, coupled with evaluation of phenotype characteristics of parental and derivative viruses, further identifies mutations responsible for such desired characteristics as attenuation, temperature sensitivity, cold-adaptation, small plaque size, host range restriction, etc.

Mutations thus identified and mapped provide a rich source of candidate mutations for designing recombinant negative stranded RNA viruses using the heterologous transfer methods described herein. Exemplary disclosures which identify and characterize such attenuating mutations in negative stranded RNA viruses are provided in U.S. patent application Ser. No. 08/892,403 and corresponding International Application No. WO 98/02530, and U.S. patent application Ser. No. 08/083,793 and corresponding International Application No. WO 98/53078. These and other references incorporated herein provide a “menu” of attenuating mutations that are candidates for transfer into heterologous viral clones according to the methods of the invention.

For example, U.S. patent application Ser. No. 08/892,403 and corresponding International Application No. WO 98/02530 describe cold passaged (cp) and temperature sensitive (ts) mutants of RSV (subfamily Pneumovirinae; genus Pneumovirus), including the exemplary mutants designated cpts RSV 248 (ATCC VR 2450), cpts RSV 248/404 (ATCC VR 2454), cpts RSV 248/955 (ATCC VR 2453), cpts RSV 530 (ATCC VR 2452), cpts RSV 530/1009 (ATCC VR 2451), cpts RSV 530/1030 (ATCC VR 2455), RSV B-1 cp52/2B5 (ATCC VR 2542), and RSV B-1 cp-23 (ATCC VR 2579. From these biologically-derived mutants an exemplary panel of attenuating mutations has been mapped and characterized, including specific nucleotide changes in the large polymerase gene L resulting in amino acid substitutions at parental residue/sequence positions Phe₅₂₁, Gln₈₃₁, Met₁₁₆₉, and Tyr₁₃₂₁, as exemplified by the attenuating substitutions, Leu for Phe₅₂₁, Leu for Gln₈₃₁, Val for Met₁₁₆₉, and Asn for Tyr₁₃₂₁. Each of these mutations occurs in the highly conserved L protein and confers a ts phenotype on the mutant virus. However, additional mutations have been identified in RSV and other negative stranded RNA viruses, as well as in other conserved proteins, which confer a range of attenuating phenotypes including ts and non-ts attenuating phenotypes eg., as present in cold passaged (cp) small plaque (sp), cold-adapted (ca) or host-range restricted (hr) mutant strains.

Thus, an additional menu of exemplary mutations for incorporation within recombinant negative stranded viruses according to the methods of the invention has been identified for the distantly related paramyxovirus, human PIV3 (subfamily Paramyxovirinae; genus Respirovirus). One such panel of mutations has been identified and characterized in the biologically-derived (cold-passaged) HPIV3 mutant virus strain JS cp45 (see, U.S. patent application Ser. No. 08/083,793 and corresponding International Application No. WO 98/53078, incorporated herein by reference). Among the mutations mapped and characterized within this strain are nucleotide changes encoding is attenuating amino acid substitutions in the polymerase L gene at parental residue/sequence positions Tyr₉₄₂, Leu₉₉₂, and/or Thr₁₅₅₈. In the exemplary JS cp45 mutant L protein, Tyr₉₄₂ is replaced by His, Leu₉₉₂ is replaced by Phe, and Thr₁₅₅₈ is replaced by Ile. These mutations have been successfully incorporated in various PIV recombinants, including the recombinants designated as r942, r992, r1558, r942/992, r992/1558, r942/1558, and r942/992/1558 incorporating the numerically indicated mutations singly and in combination.

Other exemplary mutations identified in HPIV3 JS cp45 have been mapped and characterized to encode attenuating amino acid substitutions in the F and C proteins of HPIV3. These include mutations encoding non-ts attenuating amino acid substitutions in the C protein of the P gene at the parental residue/position Ile₉₆ of JS HPIV3, as exemplified by the substitution of Ile₉₆ to Thr. Further exemplary mutations identified in the F protein of HPIV3 encode amino acid substitutions at parental residue/positions Ile₄₂₀ and Ala₄₅₀, as exemplified by the substitutions Ile₄₂₀ to Val and Ala₄₅₀ to Thr.

Also identifiable in this manner are attenuating mutations in non-coding portions of a negative stranded viral gene. For example, attenuating mutations may include single or multiple base changes in a gene start sequence, as exemplified by an attenuating base substitution in the RSV M2 gene start sequence at nucleotide 7605. Where such mutations map to conserved nucleotide positions within a heterologous negative stranded RNA virus, they also are amenable to transfer between heterologous taxa according to the methods of the invention.

Each attenuating mutation thus identified in a negative stranded RNA virus provides an index for sequence comparison against a homologous protein in one or more heterologous negative stranded virus. To practice this aspect of the invention, existing sequence alignments may be analyzed, or conventional sequence alignment methods may be employed to conduct sequence comparisons to identify corresponding protein regions and amino acid positions between a protein bearing an identified attenuating mutation in one negative stranded RNA virus and a homologous protein in a different virus that is the target virus for recombinant attenuation. The focus of this exercise is to identify one or more residues that are associated with the attenuating mutation in the first (heterologous) virus, i.e., which has been altered from a parental sequence where the parent lacks the mutant phenotype. By sequence alignment it is then determined whether the parental sequence of the mutant is conserved, by the presence of an identical or conservative amino acid residue at a corresponding amino acid position(s) in the target (recombinant) virus protein. Typically, the “wild-type” sequence element(s) thus conserved will occur in a conserved region or domain of the protein, however isolated residues and blocks of amino acid residues are also widely conserved among different taxa within the Mononegavirales and can provide equally useful targets for heterologous transfer of attenuating mutations between heterologous RNA viruses (wherein all or part of the conserved sequence element(s) bearing the mutant change is copied or imported into the recombinant virus to yield a novel attenuated derivative).

Various conserved proteins among the Mononegavirales provide useful targets within the invention for introducing attenuating mutations found or generated in one, heterologous negative stranded RNA virus to a different, recombinant virus. This is attributable to the remarkable degree of structural and functional conservation among various taxa within the Mononegavirales. Throughout this order, five target proteins are universally conserved as accepted homologous proteins derived from a distant common ancestral virus. These proteins typically show moderate to high degrees of sequence identity, particularly within specific regions or domains that are postulated to share common functional attributes. Specifically, all known negative stranded RNA viruses share a homologous constellation of proteins comprising a nucleocapsid protein (N), a nucleocapsid phosphoprotein (P), a nonglycosylated matrix (M) protein, at least one surface attachment glycoprotein (HN, H, or G) and a large polymerase (L) protein. These proteins each exhibit conserved sequence elements that represent useful targets for transferring attenuating mutations by alteration of one or more conserved residues shared between a target virus and a heterologous parental virus for which mutant derivatives or constructs have been identified to exhibit an amino acid change that specifies an attenuated phenotype. Additional proteins are also targeted in this manner that are only shared among particular families, subfamilies, genera or species within the Mononegavirales.

The Order Mononegavirales embraces the families Filoviridae, Paramyxoviridae, Bornaviridae and Rhabdoviridae, which are all comprised of viruses with monopartite negative-stranded RNA genomes, Pringle, Arch Virol. 117:137-140 (1991). A summary of the taxonomy within this group, including identification of family/subfamily/generic and type specific groupings is provided by Pringle, Arch Virol. 142(11): 2321-2326 (1997). A representative classification of the Mononegavirales, and expanded classification of the Paramyxoviridae, is set forth herein in Table 1. Common features apparent in the genetic organization of these three families of viruses with linear negative stranded RNA genomes justify their grouping together as an order, particularly in view of the fact that genetic recombination occurs rarely, if at all, in these viruses and consequently phenotypic relationships are likely to reflect genetic continuity.

TABLE 1
Nonsegmented Negative Strand RNA Animal Viruses

```
Order Mononegavirales
  Family Rhabdoviridae
    Genus Vesiculovirus (vesicular stomatitis virus)
    Genus Lyssavirus (rabies virus)
    Genus Ephemerovirus (bovine ephemeral fever virus)
    Other genera
  Family Filoviridae (Ebola virus, Marburg virus)
  Family Bornaviridae (Borna disease virus)
  Family Paramyxoviridae
Classification of Respiratory Syncytial Virus within
Family Paramyxoviridae and Members of the Indicated Taxa.
Subfamily Paramyxovirinae
  Genus Respirovirus
    Sendai virus (murine parainfluenza virus type 1)
    human parainfluenza virus type 1
    human parainfluenza virus type 3
    bovine parainfluenza virus type 3
  Genus Morbillivirus
    measles virus
    canine distemper virus
    rinderpest virus
    cetacean (dolphin) Morbillivirus
    phocine (seal) distemper virus
    pest-des-petits-ruminants virus
    Hendra virus (newly identified Australian pathogen)
  Genus Rubulavirus
    mumps virus
    simian virus 5 (canine parainfluenza virus type 2)
    human parainfluenza virus type 2
    human parainfluenza virus type 4
    avian parainfluenza virus (including Newcastle disease virus)
    porcine Rubulavirus
    simian virus type 41
    Mapuera virus
Subfamily Pneumovirinae
  Genus Pneumovirus
    human respiratory syncytial virus (subgroups A and B)
    bovine respiratory syncytial virus
    ovine and caprine respiratory syncytial virus
    pneumonia virus of mice
  Genus avian pneumovirus¹
    avian pneumovirus (formerly turkey rhinotracheitis virus)
¹Assignment as a separate genus, yet to be named, is planned.
```

The Families Bornaviridae and Filoviridae are represented by single genera. The Genus Bornavirus as yet contains only a single species (borna disease virus). Four species are recognized in the Genus Filovirus (type species Marburg virus) by virtue of nucleotide sequence and antigenic divergence and differential expression of the attachment (G) protein. The Family Rhabdoviridae includes five genera, Vesiculovirus, Lyssavirus, Ephemerovirus, Cytorhabdovirus, and Nucleorhabdovirus, which in addition to sequence and antigenic differences are distinguished by host range, presence of supplementary genes, and intracellular site of multiplication. The Family Paramyxoviridae is represented by two sub-families; the Paramyxovirinae with three genera, Respirovirus (type species HPIV1), Morbillivirus (type species MeV), and Rubulavirus (type species MuV), and the Pneumovirinae (type species human RSV) and one planned additional genus which will include avian pneumovirus.

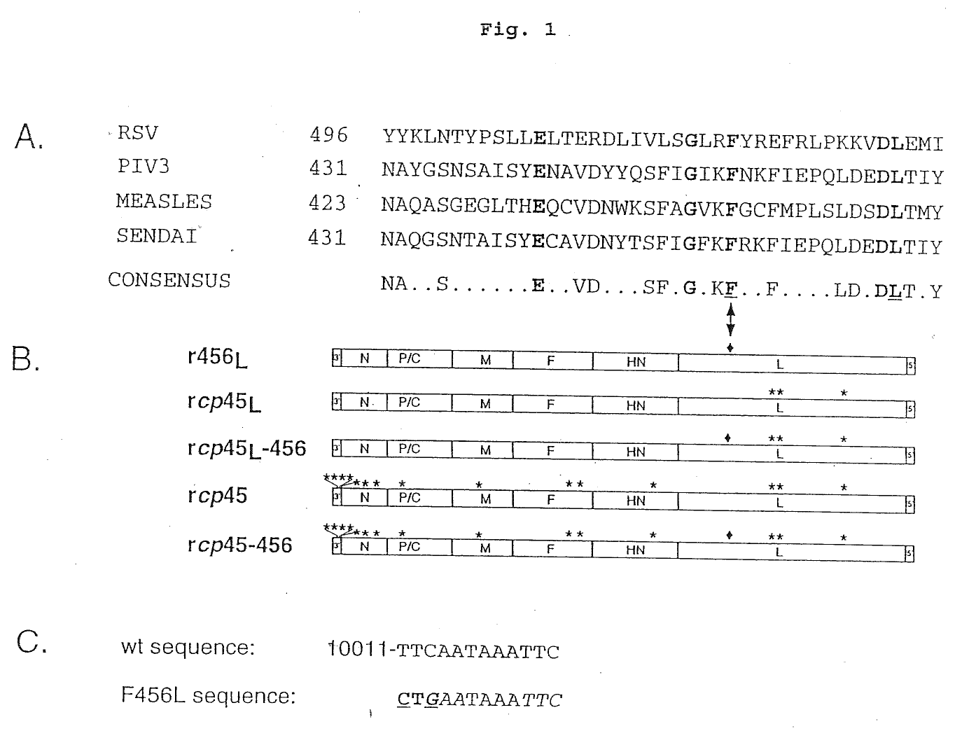

In all members of the order there are 5-10 genes and transcription is initiated from a single presumptive 3′-terminal promoter by a viral RNA-dependent RNA polymerase. With the exception of some pneumoviruses there is strict conservation of gene order. In FIG. 1 of Pringle, Arch Virol. 142(11): 2321-2326 (1997) genes are compared for sixteen viruses representing different families, subfamilies, and genera within the Mononegavirales, which genes are classified and grouped as homologs between the various taxa described. Thus, VSV is shown to exhibit the minimal complement of five genes; nucleoprotein (N), phosphoprotein (P), matrix protein (M), attachment protein (G), and polymerase protein (L). Borna disease virus of the Bornaviridae, Ebola virus of the Filoviridae and eight members of the Rhabdoviridae exhibit a basic five gene pattern (N-P-M-G-L), augmented in the case of Ebola virus and four of the eight rhabdoviruses by insertion of one or more genes between G and L, or between P and M in the case of three of the remaining four rhabdoviruses. Paramyxoviruses of the Subfamily Paramyxovirinae exhibit an increase in complexity; the basic 5 gene pattern is enhanced by multiple encoding of genetic information in the P gene and the insertion of an additional envelope protein gene (F) between M and the attachment protein (H or HN), and in addition by SH in certain of the Rubulaviruses. All of the paramyxoviruses have at least two surface glycoproteins, HN (or H or G) and F. Almost all members of the Respirovirus, Rubulavirus and Morbillivirus genera have a cysteine-rich protein V. Respiroviruses and Morbilliviruses also encode homologous C proteins. Pneumoviruses and a subset of Rubulaviruses (Simian virus 5 (SV5) and mumps virus (MuV)) share homologous surface glycoproteins SH. Within the Pneumovirus genus (including bovine, ovine and caprine RSV and pneumonia virus of mice—alternatively referred to herein as murine RSV) several additional proteins, NS1, NS2, M2(ORF1) and M2(ORF2) are conserved. The paramyxoviruses of the subfamily Pneumovirinae, exhibit unique deviations from the basic pattern, the most significant being possession of additional genes. In the avian, human and murine pneumoviruses the M2 gene encodes two proteins in different reading frames, both of which have significant effects on RNA synthesis in vitro. In the human and murine pneumoviruses two genes encoding non-structural proteins NS1 and NS2 of unknown function are located between the 3′-leader region and N. The avian pneumovirus is closest to the basic pattern, lacking the two unique 3′ terminal NS genes and retaining the standard paramyxovirus gene order with the exception of insertion of the M2 gene. This conservative pattern of genome organization suggests evolution by expansion of intergenic regions and by gene duplication, rather than by introduction of genetic information from outside. Pringle, Semin. Virol. 8:49-57, 1997.

As noted above, the methods of the invention are based on identification of an attenuating mutation in a first negative stranded RNA virus, which mutation is mapped by comparison of a mutant versus a wild-type sequence to yield identification of a subject amino acid position(s) marking the site of the mutation. Preferably, these mutations are incorporated into a non-attenuated or partially attenuated, recombinant viral clone to verify that the subject change in fact specifies an attenuated phenotype. A host of exemplary attenuating mutations in this context are provided in the instant disclosure, and others are readily identifiable in accordance with known mutagenic and reverse genetics techniques in accordance with the description herein. Each attenuating mutation identified in one negative stranded RNA virus provides an index for sequence comparison against a homologous protein in one or more heterologous negative stranded virus(es).

To practice the above aspects of the invention, existing sequence alignments may be analyzed, or conventional sequence alignment methods may be employed to yield sequence comparisons for analysis, to identify corresponding protein regions and amino acid positions between the protein bearing the attenuating mutation and a homologous protein of a different virus that is the target recombinant virus for attenuation. Where one or more residues marking the attenuating mutation have been altered from a parental, eg., wild-type, identity that is conserved (i.e., identical or represented by a conservatively related amino acid residue) at the corresponding amino acid position(s) in the target virus protein, the genome or antigenome of the target virus is recombinantly modified to encode an identical or conservative amino acid deletion, substitution, or insertion to alter the conserved residue(s) in the target virus protein and thereby confer an analogous, attenuated phenotype on the recombinant virus.

Sequence alignments and analysis to practice this aspect of the invention focus on homologous, “counterpart” genes, gene segments, proteins and/or protein domains in which a polynucleotide or amino acid reference sequence is used as a defined sequence to provide a basis for statistical sequence comparison. For example, the reference sequence may be a defined segment of a cDNA or gene, a complete cDNA or gene sequence or a sequence of a protein or sub-portion thereof.

Generally, a reference sequence, for use in defining counterpart genes or gene segments, is at least 20 nucleotides in length, frequently at least 25 nucleotides in length, and often at least 50 nucleotides in length and that for proteins and protein segments is at least 20 amino acids in length. Since two polynucleotides or amino acid sequences may each (1) comprise a sequence (i.e., a portion of the complete polynucleotide or protein sequence) that is similar between the two polynucleotides or proteins, and (2) may further comprise a sequence that is divergent between the two polynucleotides or proteins, sequence comparisons between two (or more) polynucleotides or proteins are typically performed by comparing sequences of the two subject sequences over a “comparison window” to identify and compare local regions of sequence identity or similarity. A “comparison window”, as used herein, refers to a conceptual segment of at least 20 contiguous nucleotide or amino acid positions wherein a sequence may be compared to a reference sequence of at least 20 contiguous nucleotides or amino acids, and wherein the portion of the polynucleotide or amino acid sequence in the comparison window may comprise additions or deletions (i.e., gaps) of 20 percent or less as compared to the reference sequence (which does not comprise additions or deletions) for optimal alignment of the two sequences.

Optimal alignment of sequences for aligning a comparison window may be conducted by the local homology algorithm of Smith & Waterman, Adv. Appl. Math. 2:482 (1981), by the homology alignment algorithm of Needleman & Wunsch, J. Mol. Biol. 48:443 (1970), by the search for similarity method of Pearson & Lipman, Proc. Natl. Acad. Sci. USA 85:2444 (1988) (each of which is incorporated by reference), by computerized implementations of these algorithms (GAP, BESTFIT, FASTA, and TFASTA in the Wisconsin Genetics Software Package Release 7.0, Genetics Computer Group, 575 Science Dr., Madison, Wis., incorporated herein by reference), or by inspection, and the best alignment (i.e., resulting in the highest percentage of sequence similarity over the comparison window) generated by the various methods is selected.

The term “sequence identity” as used herein means that two polynucleotide or protein sequences are identical (i.e., on a nucleotide-by-nucleotide or residue-by-residue basis) over the window of comparison. The term “percentage of sequence identity” is calculated by comparing two optimally aligned sequences over the window of comparison, determining the number of positions at which the identical nucleic acid base (e.g., A, T, C, G, U, or J) or amino acid residue occurs in both sequences to yield the number of matched positions, dividing the number of matched positions by the total number of positions in the window of comparison (i.e., the window size), and multiplying the result by 100 to yield the percentage of sequence identity.

The term “substantial identity” as used herein denotes a characteristic of two polynucleotide or amino acid sequences which share at least 85 percent sequence identity, preferably at least 90 to 95 percent sequence identity, and sometimes at 99 percent or greater sequence identity over a comparison window of at least 20 nucleotide or amino acid positions, frequently over a window of at least 25-50 nucleotides or amino acids, wherein the percentage of sequence identity is calculated by comparing the reference sequence to a comparison sequence which may include deletions or additions which total 20 percent or less of the reference sequence over the window of comparison. The reference sequence may be a subset of a larger sequence.

As applied to proteins and structural elements within proteins, the term “sequence identity” means that the sequences share one or more identical amino acids at corresponding positions. The term “sequence similarity” means that two sequences are share one or more conservatively related amino acids at corresponding positions, eg., attributable to conservative substitutions. The term “substantial sequence identity” means that two peptide sequences, when optimally aligned, such as by the programs GAP or BESTFIT using default gap weights, share at least 85 percent sequence identity, preferably at least 90 percent sequence identity, more preferably at least 95 percent sequence identity or more (e.g., 99 percent sequence identity). The term “substantial similarity” means that two peptide sequences share corresponding percentages of sequence similarity.

Conservative sequence relationships exist even when amino acid residues at corresponding positions between two sequences are not identical but differ by a conservative amino acid structural relationship. In this context, conservative amino acid substitutions refer to the general interchangeability of amino acid residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. Stereoisomers (e.g., D-amino acids) of the twenty conventional amino acids, unnatural amino acids such as α,α-disubstituted amino acids, N-alkyl amino acids, lactic acid, and other unconventional amino acids may also be suitable components for polypeptides of the present invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). Moreover, amino acids may be modified by glycosylation, phosphorylation and the like.

To facilitate practice of the above aspects of the invention, reference is made to the known molecular phylogeny of conserved viral proteins within the order Mononegavirales. In this regard, extensive studies have provided detailed assessments of protein relatedness on a precise, molecular level. These studies include detailed sequence comparisons for conserved proteins among the Mononegavirales, yielding widely accepted maps of conserved structural domains, sequence elements and constrained, isolated residues within these proteins. Each of these conserved protein domains, sequence elements and constrained residues facilitate practice of the invention by providing useful targets for conservative transfer of attenuating mutations from a heterologous, attenuated mutant virus to a different, recombinant virus according to the methods of the invention.

For example, Poch et al., J. Gen. Virol. 5:1153-62, (1990) (incorporated herein by reference) provide a detailed comparison of the deduced amino acid sequences for five L proteins within the Mononegavirales, including L proteins of rhabdoviruses (VSV and RaV) and paramyxoviruses (SeV, NDV and MeV). Conventional alignment methods (incorporated herein by reference) reveal that these L proteins from diverse virus families exhibit a high degree of homology along most of their length—with strongly invariant amino acids embedded in conserved blocks separated by regions which are relatively all well conserved. Thus, these different L proteins from heterologous negative stranded RNA viruses possess a conservative structure of concatenated functional domains. For example, the L protein includes a highly conserved central region which is thought to contain the active site for RNA synthesis. Other conserved structural domains, motifs and sequence elements, including stringently conserved, isolated residues are also identified, some of which are distributed around the conserved central core and are thought to be important for polymerase activity. These conserved sequence elements identified in the L protein (see, Poch et al., J. Gen. Virol. 5:1153-62, (1990) and Poch et al. EMBO J. 8(12)3867-3874, (1989), particularly FIG. 1, incorporated herein by reference), provide detailed information to compare with L protein sequences of heterologous, parental strains from which attenuating mutations have been generated and mapped. Thus, using these and other sequence methods and data, including the sequence methods and data presented in the publications incorporated herein, it can be readily determined whether a residue(s) marking an attenuating mutation has been altered from a parental, eg., wild-type, identity that is conserved (i.e., identical or represented by a conservatively related amino acid residue) at the corresponding amino acid position(s) in a target virus L protein—which determination indicates a high probability for successful incorporation of the attenuating mutation identified in the heterologous virus into the antigenome or genome of the recombinant, target virus.

Further detail concerning structural conservation among different L protein homologs throughout the Mononegavirales is provided in Stec et al., Virology 183:273-287 (1991), incorporated herein by reference. This sequence analysis is based on published sequences of L genes and proteins for three paramyxoviruses, two from the genus Respirovirus (PIV3 and SeV), and one from the genus Rubulavirus (NDV), one paramyxovirus from the genus Morbillivirus (MeV), and two viruses from the family Rhabdoviridae (RaV and VSV) (see eg., Schubert et al., 1985; Shioda et al., 1986; Yusoff et al., 1987; Blumberg et al., 1988; Galinski et al., 1988; and Teart et al., 1988, each incorporated herein by reference), and provides additional sequence information and alignment results for RSV. These results confirm the teachings of Poch et al., supra, regarding the highly significant sequence conservation within the paramyxovirus and rhabdovirus families, and identify yet additional conserved structural domains, motifs and sequence elements.

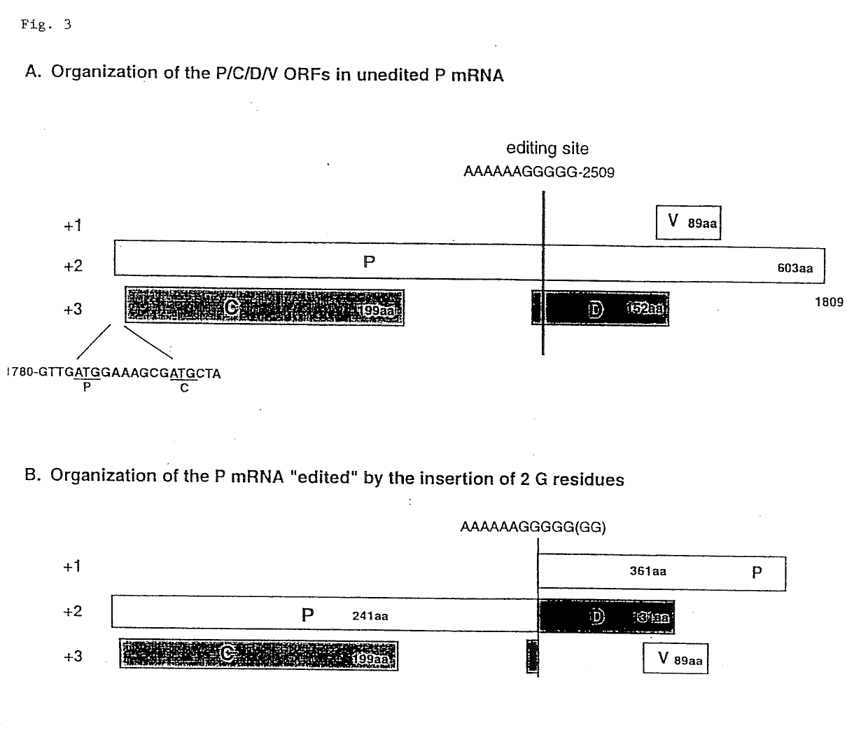

Briefly, the disclosure of Stec et al. relies on conventional alignment methods which, among other similar methods, are useful within the instant invention. In particular, Stec et al. aligned heterologous negative stranded RNA viral sequences using the accepted methods of Wilbur and Lipman Proc. Natl. Acad. Sci. USA 80:726-730 (1983), using deletion and gap penalties of 12 and 6, respectively, and the similarity scoring matrix of Dayhoff et al., in “Atlas of Protein Sequence and Structure” (M. O. Dayhoff, Ed.), Vol. 5, Suppl. 3, pp. 345-352 Natl. Biomed. Res. Found., Silver Spring, Md. (1978), each incorporated herein by reference. The similarity scoring system was used to construct pairwise global dot matrix alignments between RSV and other negative-strand RNA virus L proteins listed in Table 1. By these methods, regions of unambiguous sequence relatedness were detected between the RSV L protein and the L proteins of distantly related paramyxoviruses and rhabdoviruses. As illustrated in FIG. 3, the regions of sequence relatedness between the RSV L protein and each of the others are colinear and concentrated in the amino-proximal region, representing approximately one-fifth of each molecule. The same pattern of sequence similarity was noted in previous alignments of the other paramyxovirus and rhabdovirus L proteins (Blumberg et al., 1988, Galinski et al., 1988; Teart et al., 1988, each incorporated herein by reference), including the five-way alignment of SeV, MeV, NDV, RaV, and VSV L proteins conducted by Poch et al. (1989), supra.

In the seven-way alignment of Stec et al., supra, the RSV L protein was determined to contain an amino-terminal extension of about 70 amino acids and a carboxy-terminal truncation of about 100 amino acids relative to other negative stranded RNA viruses. However, this change does not affect the reliability of alignment methods to identify conservative structural elements, and is thought to be attributable to the fact that the RSV L protein is encoded, in part, by the overlap of the L gene with its upstream neighbor, the M2 gene (previously called 22K) (Collins et al., 1987, incorporated herein by reference).

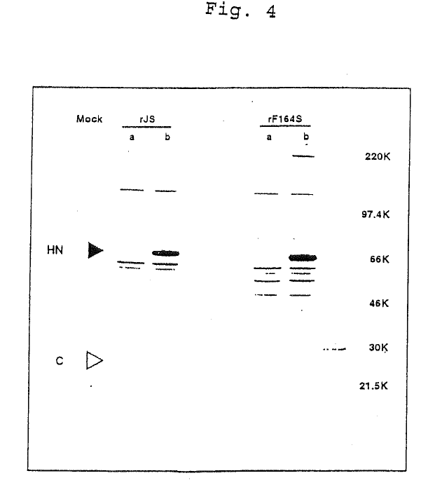

The disclosure of Stec et al., supra, confirms the presence of a number of short segments in the L proteins of heterologous negative stranded RNA viruses that are almost exactly conserved between different taxa. These nearly identical segments are also shown to be conserved in the RSV L protein, and reside within the highly conserved region shown in FIG. 4 of Stec et al. (incorporated herein by reference, see boxed sequences). The amino acid identity within these six segments varies from 30 to 80% among the seven proteins. Numerous residues within the identified segments are invariant among the seven different negative stranded RNA viruses for which the alignment was conducted. Moreover, it is noteworthy that where substitutions have occurred in these conserved segments, many are marked by conservative substitutions. As noted previously, the high degree of identity of these segments among each member of two virus families suggests that they are important for L protein functions.

In more specific detail, the most highly conserved region of the L proteins aligned between divergent taxa within the Mononegavirales in FIG. 4 of Stec et al., also contains four distinct polymerase motifs (underlined and designated A-D) consistent with the motifs identified by Poch et al., (1989), supra. These regions homologous to the four polymerase motifs were identified in the RSV L protein between amino acids 696 to 887. Three of the four elements (A-C) coincide with the highly conserved segments of the paramyxovirus and rhabdovirus L proteins described above. The D segment was less well conserved among these viruses but does contain a single invariant lysine and a conserved glycine (in RSV, K-886 and G-877, respectively) that typifies this element.

Similarly detailed sequence alignments and analyses have been published for other proteins shared among the negative stranded RNA viruses and useful as target proteins within the instant invention. In this regard, Barr et al., J. Gen. Virol. 72:677-685 (1991), incorporated herein by reference, analyzed sequence conservation in the N protein among heterologous members of the Mononegavirales. In particular, this study showed a high level of amino acid identity (60%) between the predicted amino acid sequences of RSV and PVM N proteins (see FIG. 7, incorporated herein by reference). Amino acid residues 1 to 150 and 150 to 393 contain 38% and 74% identity respectively, whereas residues 245 to 315 contain 68 identical amino acids out of 71 (96% identity). This high degree of conservation of these proteins is consistent with observations that the N proteins of RSV and PVM are serologically related (Gimenez et al., 1984; Ling & Pringle, 1989).

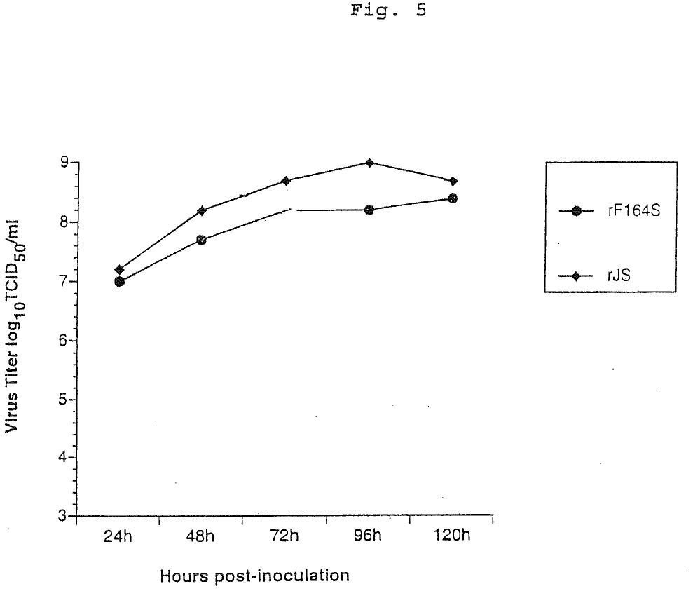

Computer matrix comparisons of the amino acid sequences of the N proteins of PVM and more divergent members of the Mononegavirales also reveal regions of conservation (Barr et al., supra, FIG. 5). Sequence conservation in this context extends as far as between the N proteins of PVM and Ebola virus. Furthermore, hydropathy profiles of N proteins from members of widely diverse groups of non-segmented negative-strand viruses resemble each other in the region of greatest sequence similarity between paramyxovirus and Morbilliviruses (Galinski et al., 1985, incorporated herein by reference). Approximately 180 amino acids, commencing 130 to 170 residues from the amino terminus of each protein form alternating hydrophobic and hydrophilic regions. Secondary structure predictions for these amino acid sequencers (Garnier et al., 1978, incorporated herein by reference) suggest that this region of conserved hydropathy may commence with a high proportion of α-helix but that it terminates with a high proportion of β-sheet and reverse turn. These data suggest that these conserved proteins may have a similar folded structure over the region of similar hydropathy.

Further alignment studies of representative N proteins focusing on the region of conserved hydropathy using the well known program CLUSTAL (Higging & Sharp, Gene 33:237-244 (1988), incorporated herein by reference) point to yet additional conserved protein domains, structural motifs and isolated, conserved amino acid residues (see, Barr et al., FIG. 7, boxes A, B, and C). From this study, it was found that conservation among the SeV, VSV and PVM N proteins is particularly high in a defined region within the carboxy-terminal half of the PVM sequence (FIG. 7, box C). This region was also identified in DIAGON comparisons between Ebola virus and PVM with a window smaller than 99. Two short regions of similarity were also been detected within the sequences, each composed of hydrophobic regions interrupted by a single, conserved, basic amino acid (K or R: boxes A and B in FIG. 7). Corresponding regions are similarly spaced in other paramyxovirus and rhabdovirus N proteins. The levels of identity in these regions are described in Table 1 of Barr et al. Although the highest proportions of identities are seen within a virus family, high levels of similarity are demonstrable in these regions between virus families, particularly when conservative substitutions are taken into account.

Also facilitating practice of the invention are independent published alignments confirming the high degree of molecular conservation of N proteins between heterologous members of the Mononegavirales. For example, Parks et al., Virus Res. 22:259-279, (1992), incorporated herein by reference, describe computer-assisted alignment of amino acid sequences of N proteins from 10 different paramyxoviruses. As shown in FIG. 3 of the reference, a distinct region near the middle of the N protein (SV5 residues 323-340) contains a large stretch of sequence conservation among N proteins of different taxa, including an invariant hydrophobic motif F-X4-Y-X4-S-Y-A-M-G (where X is any residue). A second highly conserved domain, which is enriched in the negatively charged amino acids glutamate and aspartate, was identified in the C-terminal region of the N proteins (SV5 residues 455-469).

In another study of N protein molecular conservation, Miyahara et al., Arch. Virol. 124:255-268 (1992), incorporated herein by reference, compared the amino acid sequence of the HPIV-1 N protein with corresponding sequences of 12 other paramyxoviruses, HPIV-2 (Yuasa et al., Virology 179:777-784 (1990)); HPIV3 (Galinski et al., Virology 149:139-151 (1986)), HPIV-4A and -4B (Kondo et al., Virology 174:1-8 (1990)), SV5 and SV41 (Tsurudome et al., J. Gen. Virol. 72:2289-2292 (1991)), MuV (Elango, Virus Res. 12:77-86 (1989)), SeV (Morgan et al., Virology 135:279-287 (1984)), PIV-3 (Sakai et al., Nucleic Acids Res. &:2927-2944 (1987)), NDV (Ishida et al., Nucleic Acids Res. 14:6551-6564 (1986)), MeV, and (CDV) (Rozenblatt et al., J. Virol. 53:684-690 (1985)) (each of the foregoing references incorporated herein by reference.

As shown in FIG. 3 of Miyahara et al., the N gene of HPIV1 shows extensive homology with SeV; the nucleotide and amino acid identities were 70.8% and 87.8%, respectively. The N protein of HPIV-1 also showed high amino acid identities with HPIV-3 (63.1%) and BPIV-3 (63.3%), and lesser identities to NDV (20.9%), MeV (20.5%), CDV (19.6%), SV41 (18.6%), SV5 (17.9%), HPIV-4A (17.9%), HPIV-4B (17.5%), HPIV-2 (17.5%) and MuV (17.1%). A protease-sensitive “hinge” was found at the junction of two defined domains of the SeV K protein; (1) an amino-terminal domain which interacts directly with the RNA, and (2) a carboxyl-terminal domain which lies on the surface of the assembled nucleocapsid (Heggeness et al., Virology 114:555-562 (1981), incorporated herein by reference)

As further described in Miyahara et al., twenty-six amino acids were conserved in all the paramyxovirus N proteins, and a conserved N domain was identified in the N protein between amino acids 260-360. In addition, thirty-seven out of 40 glycines and 13 out of 13 prolines were conserved in the N proteins of HPIV-1 and SV, suggesting that these proteins SeV maintain a common tertiary structure.

Miyahara also compared M protein sequences between the HPIV-1 M protein and 13 other paramyxoviruses (see, eg., FIG. 4 of Miyahara et al.). These heterologous viruses also exhibited a high degree of structural conservation of M protein sequence elements. For example, HPIV-1 and SeV showed levels of nucleotide and amino acid identity for M of 72.6% and 88.4%, respectively. HPIV-1 also showed high levels of conservation with HPIV-3 (65.7%) and BPIV-3 (65.4%), and moderate conservation with MeV (36.4%), CDV (34.6%) and RPV (36.7%). All of 5 cysteines, 20 and 22 prolines and 24 of 25 glycines of HPIV-1 were conserved in SeV, almost all of which were also conserved in PIV-3. These finding indicate that a tertiary structure of M protein may be conserved in HPIV-1, SeV, HPIV-3 and BPIV-3. In addition, fourteen amino acids were conserved in all the paramyxovirus M proteins compared.

Additional sequence alignments and analyses have been published for the P protein that is also universally conserved among the Mononegavirales and is thus a particularly useful target for heterologous transfer of mutations within the invention. See, eg., Kondo et al., Virology 178:321-326 (1990), incorporated herein by reference. In this context, it is noted that the P proteins of SeV (Giorgi et al., Cell 35:82-836 (1983); Neubert, Nucleic Acids Res. 17: 10-101 (1989), incorporated herein by reference) and PIV3 (Luk et al., Virology 153:318-325 (1986), incorporated herein by reference) are closely similar in size, composed of 568 and 603 amino acids, respectively. Among different taxa, eg., MuV, SV5, PIV-2 and NDV, the P gene encodes proteins containing 391, 392, 395 and 395 amino acids, respectively (Takeuchi et al., J. Gen. Virol. 69:2043-2049 (1988); Thomas et al., Cell 54:891-902 (1988); and Sato et al., Virus Res. 7:241-255 (1987)). The P proteins of MeV and CDV are intermediate in size (Barret et al., Virus Res. 3:367-372 (1985; Bellini et al., J. Virol. 53:908-919 (1985).

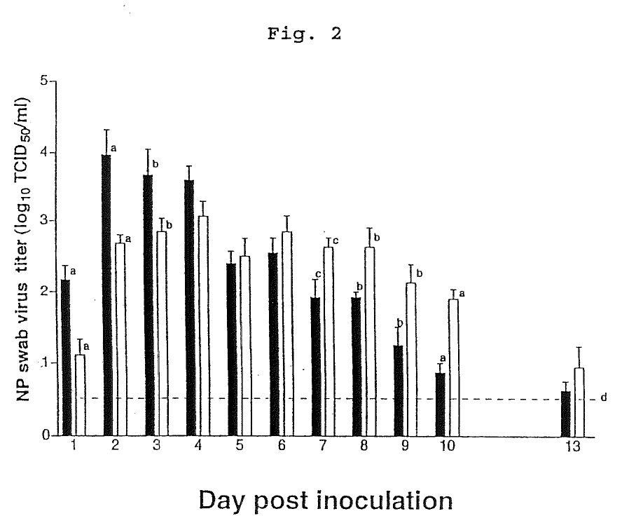

The P-specific regions of all the heterologous viruses compared by Kondo et al., Virology 178:321-326 (1990) could be aligned according to the conventional methods employed (incorporated herein by reference). Distinct conservative structural elements were identified between PIV-4A, PIV-4B, SV5, PIV-2, MuV, NDV, MeV, CDV, PIV-3 and SeV (see FIG. 2 of the Kondo paper, incorporated herein by reference). Thus, this study and other published studies of P protein molecular phylogeny identify yet additional conserved protein domains, structural motifs, amino acid segments and isolated residues for alignment with sites of mutation to evaluate targets for incorporation of attenuating mutations from heterologous, mutant viruses into recombinant vaccine strains.

Further sequence alignments and analyses are also provided which facilitate practice of the invention applied to the full range of proteins represented within the Mononegavirales. Briefly, Yuasa et al., Virology 179:777-784, (1990), incorporated herein by reference, identifies conserved structural elements in the 3′ gene end and N gene of human and non-human parainfluenza viruses, PIV-4A, PIV-4B, MuV, NDV, MeV, PIV-3, HPIV-3, SeV, and RSV (see, eg., FIGS. 2 and 4). Kawano et al., Virology, 174:308-313 (1990) provides an exemplary alignment and molecular analysis of HN proteins for eight heterologous paramyxoviruses (see, eg., FIG. 2). tsurudome et al., J. Gen. Virol. 72:2289-2292 (1991) provides an exemplary alignment and analysis for the N protein of HPIV-2, SV5, and SV41 (see, eg., FIG. 3). Spriggs et al., 1986, Virology 152:241-251 (1986) identifies structurally conserved elements in the F protein among heterologous paramyxoviruses, including RSV. Higuchi et al., J. Gen. Virol. 73:1005-1010 (1992) (see, eg., FIG. 4), Kawano et al., Nuc. Acids. Res. 19(10):2739-2746 (1991) (see, eg., FIGS. 2 and 6), Muhlberger et al, Virology 187:534-547 (1992) (see, eg., FIGS. 4 and 6), and Ogawa et al., J. Gen. Virol. 73:2743-2750 (1992) (see, eg., FIG. 3) for the L proteins and 3′ and 5′ non-coding genome ends among a large assemblage of taxa within the Mononegavirales. A more comprehensive review, which includes additional citations of references detailing molecular conservation among the N, P, C, L, M, HN, F, SH, V, D and additional ORFs and gene products within the Mononegavirales is provided by Collins et al., Fields Virology, Fields et al. eds., 3rd edition, Chapter 41:1205-1241, Lippincott-Raven, Philadelphia (1996). Each of these studies are incorporated herein by reference, specifically including their alignments and figures identifying conserved structural elements at defined positions representing target sites for incorporation of attenuating mutations within recombinant vaccine viruses according to the teachings of the invention.

In more detailed aspects of the invention, recombinant negative stranded RNA virus that has been attenuated by transfer of a mutation identified in a heterologous virus is engineered as a chimeric virus, for example a chimeric RSV or PIV virus. Chimeric negative stranded viruses of the invention are recombinantly engineered to incorporate nucleotide sequences from more than one viral strain or subgroup to produce an infectious, chimeric virus or subviral particle. In this manner, candidate vaccine viruses are recombinantly engineered to elicit an immune response in a mammalian host, including humans and non-human primates. Chimeric viruses according to the invention may elicit an immune response to a specific viral subgroup or strain, or they may elicit a polyspecific response against multiple viral subgroups or strains.

In exemplary embodiments, chimeric virus incorporating an attenuating mutation as described above may also have heterologous genes or gene segments of a heterologous virus added to or incorporated within the recombinant genome or antigenome of the subject virus, for example by substituting counterpart sequence(s) from a heterologous RSV to produce a chimeric RSV genome or antigenome. A chimeric virus of the invention thus includes a partial or complete “recipient” viral genome or antigenome from one viral strain or subgroup virus combined with an additional or replacement “donor” gene or gene segment of a different viral strain or subgroup.

In preferred aspects of the invention, the chimeric attenuated virus is an RSV comprised of a partial or complete human RSV A or B subgroup genome or antigenome combined with a heterologous gene or gene segment from a different human RSV A or B subgroup virus. To generate this recombinant virus, heterologous donor genes or gene segments from one RSV strain or subgroup is/are combined with or substituted within a recipient genome or antigenome that serves as a backbone for insertion or addition of the donor gene or gene segment. Thus, the recipient genome or antigenome acts as a vector to import and express heterologous genes or gene segments to yield chimeric RSV that exhibit novel structural and/or phenotypic characteristics. Preferably, addition or substitution of a heterologous gene or gene segment within a selected recipient RSV strain yields novel phenotypic effects, for example attenuation, growth changes, altered immunogenicity, or other desired phenotypic changes, as compared with corresponding phenotypes of the unmodified recipient and/or donor.

Exemplary, attenuated chimeric RSV have been developed and characterized which incorporate both human RSV B subgroup glycoprotein genes F and G substituted to replace counterpart F and G glycoprotein genes within an RSV A genome. This exemplary chimera has been further modified to incorporate attenuating point mutations selected from (i) a panel of mutations specifying temperature-sensitive amino acid substitutions Gln₈₃₁ to Leu, and Tyr₁₃₂₁ to Asn; (ii) a temperature-sensitive nucleotide substitution in the gene-start sequence of gene M2; and (iii) an attenuating panel of mutations adopted from cold-passaged RSV specifying amino acid substitutions Val267 Ile in the RSV N gene, and Cys319 to Tyr and His₁₆₉₀ Tyr in the RSV polymerase gene L; or (iv) a deletion of the SH gene (see, e.g., U.S. patent application Ser. No. 09/291,894). Preferably, these and other examples of chimeric viruses incorporate at least two attenuating mutations which may be derived from the same or different mutant viruses.

Exemplary, attenuated chimeric PIV have also been developed and characterized which incorporate heterologous sequences from HPIV-1 and HPIV-3, as well as attenuating mutations adopted from the PIV3 mutant, cp45, as described in U.S. Ser. No. 09/083,793, filed May 22, 1998 (corresponding to International Publication No. WO 98/53078) and its priority, provisional application filed May 23, 1997, Serial No. 60/047,575, each incorporated herein by reference. More recently, all of the attenuating mutations identified in cp45, with the exception of those in the F protein, have been successfully incorporated in an attenuated, recombinant HPIV3-1 chimera.

The introduction of heterologous immunogenic proteins, domains and epitopes to produce chimeric negative stranded RNA viruses is particularly useful to generate novel immune responses in an immunized host. Addition or substitution of an immunogenic gene or gene segment from one donor virus subgroup or strain within a recipient genome or antigenome of a different viral subgroup or strain can generate an immune response directed against the donor subgroup or strain, the recipient subgroup or strain, or against both the donor and recipient subgroup or strain. To achieve this purpose, chimeric viruses may also be constructed that express a chimeric protein, e.g., an immunogenic protein having a cytoplasmic tail and/or transmembrane domain specific to one viral strain or subgroup fused to an ectodomain of a different virus. Other exemplary recombinants of this type may express duplicate protein regions, such as duplicate immunogenic regions.

Although it is often useful to add or substitute entire genes (including cis-acting elements and coding regions) within a chimeric genome or antigenome, it is also useful to transfer only a portion of a donor gene of interest. Quite commonly, non-coding nucleotides such as cis-acting regulatory elements and intergenic sequences need not be transferred with the donor gene coding region. In addition, a variety of gene segments provide useful donor polynucleotides for inclusion within a chimeric genome or antigenome to express chimeric virus having novel and useful properties. Thus, heterologous gene segments may beneficially encode a cytoplasmic tail, transmembrane domain or ectodomain, an epitopic site or region, a binding site or region, an active site or region containing an active site, etc., of a selected protein from one virus. These and other gene segments can be added or substituted for a counterpart gene segment(s) in another virus to yield novel chimeric recombinants, for example recombinants expressing a chimeric protein having a cytoplasmic tail and/or transmembrane domain of one virus glycoprotein fused to an ectodomain of a glycoprotein of another virus. Useful genome segments in this regard range from about 15-35 nucleotides in the case of gene segments encoding small functional domains of proteins, e.g., epitopic sites, to about 50, 75, 100, 200-500, and 500-1,500 or more nucleotides for gene segments encoding larger domains or protein regions.

To construct chimeric virus bearing a transferred attenuating mutation, heterologous genes may be added or substituted in whole or in part to a background genome or antigenome to form a chimeric genome or antigenome. The mutation may be present, along with one or more additional mutations, in the heterologous gene (i.e., donor gene) or gene segment or may be introduced within the partial or complete, recipient antigenome or genome “background.” In the case of chimeras generated by substitution, a selected protein or protein region (e.g., a cytoplasmic tail, transmembrane domain or ectodomain, an epitopic site or region, a binding site or region, an active site or region containing an active site, etc.) from one virus is substituted for a counterpart gene or gene segment in a different viral genome or antigenome to yield novel recombinants having desired phenotypic changes compared to wild-type or parent strains. As used herein, “counterpart” genes, gene segments, proteins or protein regions refer to two counterpart polynucleotides from a heterologous source, including different genes in a single species or strain, or different variants of the same gene, including species and allelic variants among different viral subgroups or strains.

Counterpart genes and gene segments typically share at least moderate structural similarity. For example counterpart gene segments may encode a common structural domain of a protein of interest, such as a cytoplasmic domain, transmembrane domain, ectodomain, binding site or region, epitopic site or region, etc. Typically, they will share a common biological function as well. For example, protein domains encoded by counterpart gene segments may provide a common membrane spanning function, a specific binding activity, an immunological recognition site, etc. Counterpart genes and gene segments for use in constructing attenuated chimeric viruses within the invention embrace an assemblage of alternate species having a range of size and sequence variation. However, selection of counterpart genes and gene segments relies on substantial sequence identity between the subject counterparts, as defined hereinabove. In this context, a selected polynucleotide reference sequence is as a sequence or portion of a sequence present in either the donor or recipient genome or antigenome. This reference sequence is used as a defined sequence to provide a basis for sequence comparison. For example, the reference sequence may be a defined segment of a cDNA or gene, or a complete cDNA or gene sequence.

Well known cDNA-based methods are useful to construct a large panel of recombinant, chimeric viruses and subviral particles incorporating attenuating mutations identified in a heterologous virus. These recombinant constructs offer improved characteristics of attenuation and immunogenicity for use as vaccine agents. Among desired phenotypic changes in this context are resistance to reversion from an attenuated phenotype, improvements in attenuation in culture or in a selected host environment, immunogenic characteristics (e.g., as determined by enhancement, or diminution, of an elicited immune response), upregulation or downregulation of transcription and/or translation of selected viral products, etc.

In additional aspects of the invention, attenuated recombinant viruses incorporating an attenuating mutation identified in a heterologous virus are further modified by introducing one or more additional attenuating mutations that specify an altered attenuating phenotype. These mutations may be generated de novo and tested for attenuating effects according to a rational design mutagenesis strategy. Alternatively, the attenuating point mutations are identified in biologically-derived mutants, e.g., an RSV or PIV ts or cp mutant, and thereafter incorporated into an attenuated recombinant virus of the invention. The recombinant thus further attenuated may be a chimeric virus.

Further attenuating mutations in biologically-derived RSV for incorporation within a vaccine strain may occur naturally or may be introduced into wild-type strains by well known mutagenesis procedures as described above and in U.S. Ser. No. 08/327,263, incorporated herein by reference.

By “biologically-derived” mutant virus is meant any mutant virus not produced by recombinant means. Thus, biologically-derived mutant viruses may be of any negative stranded RNA viral species, subgroup or strain, e.g., naturally occurring RSV or PIV having a mutant genomic sequence or RSV or PIV having genomic variations from a reference wild-type sequence, e.g., having a mutation specifying an attenuated phenotype. Likewise, biologically-derived viruses include mutants derived from a parental strain by, inter alia, artificial mutagenesis and selection procedures.

Further attenuating mutations identified as described above are compiled into a “menu” and are then introduced as desired, singly or in combination, to adjust a candidate vaccine virus to an appropriate level of attenuation, immunogenicity, genetic resistance to reversion from an attenuated phenotype, etc., as desired. Preferably, recombinant mutant viruses of the invention are attenuated by incorporation of at least one, and, more preferably, two or more attenuating point mutations identified from such a menu, for example a panel of RSV mutants such as cpts RSV 248 (ATCC VR 2450), cpts RSV 248/404 (ATCC VR 2454), cpts RSV 248/955 (ATCC VR 2453), cpts RSV 530 (ATCC VR 2452), cpts RSV 530/1009 (ATCC VR 2451), cpts RSV 530/1030 (ATCC VR 2455), RSV B-1 cp52/2B5 (ATCC VR 2542), and RSV B-1 cp-23 (ATCC VR 2579). Additional mutations may be incorporated from conspecific or heterologous viruses, including viruses having ts, Cp, or non-ts or non-cp attenuating mutations as identified, e.g., in small plaque (sp), cold-adapted (ca) or host-range restricted (hr) mutant strains. Attenuating mutations may be selected in coding portions of genes or in non-coding regions such as a cis-regulatory sequence. For example, attenuating mutations may include single or multiple base changes in a gene start sequence, as exemplified by a single base substitution in the RSV M2 gene start sequence at nucleotide 7605. In this manner, attenuation of recombinant vaccine candidates can be finely calibrated for use in one or more classes of patients, including seronegative infants. The capability of producing virus from cDNA allows for routine incorporation of these mutations, individually or in various selected combinations, into a full-length cDNA clone, whereafter the phenotypes of rescued recombinant viruses containing the introduced mutations can be readily determined.

By identifying and incorporating specific attenuating mutations associated with desired phenotypes, e.g., a Cp or ts phenotype, into infectious recombinant viral clones, the invention provides for other, site-specific modifications at, or within close proximity to, the identified mutation. Whereas most attenuating mutations produced in biologically-derived viruses are single amino acid substitutions, other “site specific” mutations can also be incorporated by recombinant techniques into recombinant viruses of the invention. As used herein, site-specific mutations include insertions, substitutions, deletions or rearrangements of from 1 to 3, up to about 5-15 or more altered nucleotides (e.g., altered from a wild-type sequence, from a sequence of a selected mutant strain, or from a parent recombinant clone subjected to mutagenesis). Such site-specific mutations may be incorporated at, or within the region of, a selected attenuating mutation. Alternatively, the mutations can be introduced in various other contexts within a viral clone, for example at or near a cis-acting regulatory sequence or nucleotide sequence encoding a protein active site, binding site, immunogenic epitope, etc. Site-specific viral mutants typically retain a desired attenuating phenotype, but may exhibit substantially altered phenotypic characteristics unrelated to attenuation, e.g., enhanced or broadened immunogenicity, or improved growth. Further examples of desired, site-specific mutants include recombinant viruses that incorporate additional, stabilizing nucleotide mutations in a codon specifying an attenuating mutation. Where possible, two or more nucleotide substitutions are introduced at codons that specify attenuating amino acid changes in a parent mutant or recombinant clone, yielding a recombinant having genetic resistance to reversion from an attenuated phenotype. In other embodiments, site-specific nucleotide substitutions, additions, deletions or rearrangements are introduced upstream (N-terminal direction with regard to the encoded viral proteins) or downstream (C-terminal direction), e.g, from 1 to 3, 5-10 and up to 15 nucleotides or more 5′ or 3′, relative to a targeted nucleotide position, e.g., to construct or ablate an existing cis-acting regulatory element.

In addition to single and multiple point mutations and site-specific mutations, changes to attenuated recombinant viruses of the invention include deletions, insertions, substitutions or rearrangements of whole genes or gene segments. These mutations may alter small numbers of bases (e.g., from 15-30 bases, up to 35-50 bases or more), or large blocks of nucleotides (e.g., 50-100, 100-300, 300-500, 500-1,000 bases) in the donor or recipient genome or antigenome, depending upon the nature of the change (i.e., a small number of bases may be changed to insert or ablate an immunogenic epitope or change a small gene segment, whereas large block(s) of bases are involved when genes or large gene segments are added, substituted, deleted or rearranged.

In additional aspects, the invention provides for supplementation of mutations adopted into a recombinant negative stranded viral CDNA from heterologous viruses, e.g., cp and ts mutations, with additional types of mutations involving the same or different genes in a further modified recombinant virus. In this regard, viral proteins can be selectively altered in terms of expression levels, or can be added, deleted, substituted or rearranged, in whole or in part, alone or in combination with other desired modifications, to yield a recombinant, attenuated virus with additional novel vaccine characteristics.

Thus, in addition to, or in combination with, attenuating mutations adopted from a heterologous viral mutant, the present invention also provides a range of additional methods for attenuating and otherwise modifying recombinant vaccine candidates based on recombinant engineering. In accordance with this aspect of the invention, a variety of alterations can be produced in an isolated polynucleotide sequence encoding the recombinant genome or antigenome. More specifically, to achieve desired structural and phenotypic changes in recombinant PIV, the invention allows for introduction of modifications which delete, substitute, introduce, or rearrange a selected nucleotide or plurality of nucleotides from a parent genome or antigenome, as well as mutations which delete, substitute, introduce or rearrange whole gene(s) or gene segment(s), within a parent genome or antigenome.

Desired modifications of attenuated recombinant viruses are typically selected to specify a desired phenotypic change, e.g., a change in viral growth, temperature sensitivity, ability to elicit a host immune response, attenuation, etc. These changes can be brought about either in a donor or recipient genome or antigenome by, e.g., mutagenesis of a parent clone to ablate, introduce or rearrange a specific gene(s) or gene region(s) (e.g., a gene segment that encodes a protein structural domain, such as a cytoplasmic, transmembrane or extracellular domain, an immunogenic epitope, binding region, active site, etc.). Genes of interest in this regard include any viral gene, e.g., 3′-NS1-NS2-N-P-M-SH-G-F-M2-L-5′ in the case of RSV, as well as heterologous genes from other viruses.

Also provided are modifications in a recombinant vaccine candidate which alter or ablate expression of a selected gene, e.g., by introducing a termination codon within a selected RSV coding sequence, changing the position of a gene relative to an operably linked promoter, introducing an upstream start codon to alter rates of expression, modifying (e.g., by changing position, altering an existing sequence, or substituting an existing sequence with a heterologous sequence) transcription signals to alter phenotype (e.g., growth, temperature restrictions on transcription, etc.), and various other deletions, substitutions, additions and rearrangements that specify quantitative or qualitative changes in viral replication, transcription of selected gene(s), or translation of selected protein(s).

The ability to analyze and incorporate other types of attenuating mutations into recombinant vaccine candidates extends to a broad assemblage of targeted changes in recombinant clones. For example, deletion of the SH gene in RSV yields a recombinant RSV having novel phenotypic characteristics, including enhanced growth. Thus, in recombinant RSV of the invention an SH gene deletion (or any other non-essential gene or gene segment deletion), is combined in a recombinant virus with one or more additional mutations specifying an attenuated phenotype, e.g., one or more point mutation(s) adopted from a heterologous virus optionally supplemented by one or more further attenuating mutations adopted from a biologically-derived attenuated mutant. In certain embodiments, the SH gene or NS2 gene of RSV is deleted in combination with one or more cp and/or ts mutations adopted from cpts248/404, cpts530/1009, cpts530/1030, or another selected mutant RSV strain, to yield a recombinant RSV having increased yield of virus, enhanced attenuation, and resistance to phenotypic reversion, due to the combined effects of the different mutations.

In the case of one exemplary SH-minus RSV clone, the modified viral genome is 14,825 nt long, 398 nucleotides less than wild-type. By engineering similar mutations that decrease genome size, e.g., in other coding or noncoding regions elsewhere in the RSV genome, such as in the P; M, F and M2 genes, the invention provides several readily obtainable methods and materials for improving chimeric RSV growth.

In addition, a variety of other genetic alterations can be produced in a recombinant genome or antigenome for incorporation into infectious viruses attenuated in accordance with the methods of the invention. Additional heterologous genes or gene segments (e.g. from different viral genes, or different viral strains or types) may be inserted in whole or in part, the order of genes changed, gene overlap removed, a genome promoter replaced with its antigenome counterpart, portions of genes removed or substituted, and even entire genes deleted. Different or additional modifications in the sequence can be made to facilitate manipulations, such as the insertion of unique restriction sites in various intergenic regions or elsewhere. Nontranslated gene sequences can be removed to increase capacity for inserting foreign sequences.

Also provided within the invention are genetic modifications in an attenuated recombinant vaccine candidate virus which alter or ablate the expression of a selected gene or gene segment without removing the gene or gene segment from the recombinant clone. For example, this can be achieved by introducing a termination codon within a selected coding sequence, changing the position of a gene or introducing an upstream start codon to alter its rate of expression, or changing transcription signals to alter phenotype (e.g., growth, temperature restrictions on transcription, etc.).

Preferred mutations in this context include mutations directed toward cis-acting signals, which can be identified, e.g., by mutational analysis of viral minigenomes. For example, insertional and deletional analysis of the leader and trailer and flanking sequences identified viral promoters and transcription signals and provided a series of mutations associated with varying degrees of reduction of RNA replication or transcription. Saturation mutagenesis (whereby each position in turn is modified to each of the nucleotide alternatives) of these cis-acting signals also has identified many mutations which reduced (or in two cases increased) RNA replication or transcription. Any of these mutations can be inserted into a recombinant antigenome or genome as described herein.

Evaluation and manipulation of trans-acting proteins and cis-acting RNA sequences using the complete antigenome cDNA is assisted by the use of viral minigenomes (see, e.g., Grosfeld et al., J. Virol. 69: 5677-5686 (1995), incorporated herein by reference), whose helper-dependent status is useful in the characterization of those mutants which are too inhibitory to be recovered in replication-independent infectious virus.

Other mutations within recombinant viruses of the present invention involve replacement of the 3′ end of the genome with its counterpart from the antigenome, which is associated with changes in RNA replication and transcription. In addition, the intergenic regions (Collins et al., Proc. Natl. Acad. Sci. USA 83:4594-4598 (1986), incorporated herein by reference) can be shortened or lengthened or changed in sequence content, and the naturally-occurring gene overlap (Collins et al., Proc. Natl. Acad. Sci. USA 84:5134-5138 (1987), incorporated herein by reference) can be removed or changed to a different intergenic region by the methods described herein.

In one exemplary embodiment, the level of expression of specific proteins, such as the RSV protective F and G antigens, can be increased by substituting the natural codon usage with one which has been designed to be consistent with efficient translation and assembled into synthetic CDNA. In this context, it has been shown that codon usage can be a major factor in the level of translation of mammalian viral proteins (Haas et al., Current Biol. 6:315-324 (1996)). Examination of the codon usage of the mRNAs encoding the F and G proteins of RSV, which are the major protective antigens, shows that the usage is consistent with poor expression. Thus, codon usage can be improved by the recombinant methods of the invention to achieve improved expression for selected genes.

In another exemplary embodiment, a sequence surrounding a translational start site (preferably including a nucleotide in the −3 position) of a selected viral gene is modified, alone or in combination with introduction of an upstream start codon, to modulate gene expression of the attenuated recombinant virus by specifying up- or down-regulation of translation.

In more specific embodiments, attenuated RSV gene expression can be modulated by altering a transcriptional GS signal of a selected gene(s) of the virus. In one exemplary embodiment, the GS signal of NS2 is modified to include a defined mutation (e.g., the 404(M2) mutation described hereinbelow) to superimpose a ts restriction on viral replication. Yet additional attenuated RSV clones can incorporate modifications to a transcriptional GE signal. For example, RSV clones may be generated which have a substituted or mutated GE signal of the NS1 and NS2 genes for that of the N gene, resulting in decreased levels of readthrough mRNAs and increased expression of proteins from downstream genes. The resulting recombinant virus will exhibit increased growth kinetics and increased plaque size, providing but one example of alteration of RSV growth properties by modification of a cis-acting regulatory element in the RSV genome.

In another example, specific expression of the G protein of an attenuated recombinant RSV is increased by modification of the G mRNA. The G protein is expressed as both a membrane bound and a secreted form, the latter form being expressed by translational initiation at a start site within the G translational open reading frame. The secreted form can account for as much as one-half of the expressed G protein. Ablation of the internal start site (e.g., by sequence alteration, deletion, etc.), alone or together with altering the sequence context of the upstream start site yields desired changes in G protein expression. Ablation of the secreted form of G also will improve the quality of the host immune response to exemplary, chimeric RSV, because the soluble form of G is thought to act as a “decoy” to trap neutralizing antibodies. Also, soluble G protein has been implicated in enhanced immunopathology due to its preferential stimulation of a Th2-biased response.

In alternative embodiments, levels of attenuated recombinant viral gene expression are modified at the level of transcription. In one aspect, the position of a selected gene in the RSV gene map can be changed to a more promoter-proximal or promotor-distal position, whereby the gene will be expressed more or less efficiently, respectively. According to this aspect, modulation of expression for specific genes can be achieved yielding reductions or increases of gene expression from two-fold, more typically four-fold, up to ten-fold or more compared to wild-type levels. In one example, the NS2 gene of RSV (second in order in the RSV gene map) is substituted in position for the SH gene (sixth in order), yielding a predicted decrease in expression of NS2. Increased expression of selected RSV genes due to positional changes can be achieved up to 10-fold or more, often attended by a commensurate decrease in expression levels for reciprocally, positionally substituted genes.

In other exemplary embodiments, viral genes may be transpositioned singly or together to a more promoter-proximal or promoter-distal site within the recombinant viral gene map to achieve higher or lower levels of gene expression, respectively. These and other transpositioning changes yield novel clones having attenuated phenotypes, for example due to decreased expression of selected viral proteins involved in RNA replication.

In more detailed aspects of the invention, attenuated recombinant viruses are further modified by ablating expression of a viral gene, for example the NS2 gene of RSV, at the translational level without deletion of the gene or of a segment thereof, by, e.g., introducing two tandem translational termination codons into a translational open reading frame (ORF). This yields viable virus in which a selected gene has been silenced at the level of translation, without deleting its gene. These forms of “knock-out” virus can exhibit reduced growth rates and small plaque sizes in tissue culture. Thus, the methods and compositions of the invention provide yet additional, novel types of attenuating mutations which ablate expression of a viral gene that is not one of the major viral protective antigens or essential for viral growth. In this context “knockout” virus phenotypes produced without deletion of a gene or gene segment can be alternatively produced by deletion mutagenesis, as described herein, to effectively preclude correcting mutations that may restore synthesis of a target protein. Methods for producing these and other knock-outs are well known in the art (as described, for example, in Kretzschmar et al., Virology 216:309-316 (1996); Radecke et al., Virology 217:418-412 (1996); and Kato et al., EMBO J. 16:178-587 (1987); and Schneider et al., Virology 277:314-322 (1996), each incorporated herein by reference).

Infectious recombinant viral clones of the invention can also be engineered according to the methods and compositions disclosed herein to enhance immunogenicity and induce a level of protection greater than that provided by infection with a wild-type or parent recombinant virus. For example, an immunogenic epitope from a heterologous viral strain or type, e.g., PIV, can be added to a recombinant clone, e.g., RSV, by appropriate nucleotide changes in the polynucleotide sequence encoding the chimeric genome or antigenome. Alternatively, viruses can be engineered to add or ablate (e.g., by amino acid insertion, substitution or deletion) immunogenic epitopes associated with desirable or undesirable immunological reactions.

Within the methods of the invention, additional genes or gene segments may be inserted into or proximate to a recipient genome or antigenome. These genes may be under common control with recipient genes, or may be under the control of an independent set of transcription signals. Genes of interest include those encoding cytokines (e.g., IL-2 through IL-15, especially IL-2, IL-6 and IL-12, etc.), gamma-interferon, and proteins rich in T helper cell epitopes. These additional proteins can be expressed either as a separate protein, or as a chimera engineered from a second copy of one of the viral proteins, such as SH. This provides the ability to modify and improve the immune responses against the virus both quantitatively and qualitatively.

In exemplary embodiments of the invention, insertion of foreign genes or gene segments, and in some cases of noncoding nucleotide sequences, within a recombinant viral genome results in a desired increase in genome length causing yet additional, desired phenotypic effects. Increased genome length results in attenuation of the resultant virus, dependent in part upon the length of the insert. In addition, the expression of certain proteins, e.g. a cytokine, from a heterologous source into recombinant attenuated viruses of the invention will result in further attenuation of the virus due to the action of the protein. This has been described for IL-2 expressed in vaccinia virus (e.g. Flexner et al., Nature 33:-259-62 (1987)) and is also expected for gamma interferon.

Deletions, insertions, substitutions and other mutations involving changes of whole viral genes or gene segments within recombinant viruses of the invention yield highly stable vaccine candidates, which are particularly important in the case of immunosuppressed individuals. Many of these changes will result in attenuation of resultant vaccine strains, whereas others will specify different types of desired phenotypic changes. For example, certain viral genes are known which encode proteins that specifically interfere with host immunity (see, e.g., Kato et al., EMBO. J. 16:578-87 (1997), incorporated herein by reference). Ablation of such genes in recombinant vaccine viruses is expected to reduce virulence and pathogenesis and/or improve immunogenicity.

Additional mutations to further attenuate recombinant viruses of the invention include introduction of heterologous genes or cis-acting elements that confer host range restriction and other desired phenotypes favorable for vaccine use. In exemplary embodiments, bovine RSV sequences are selected for introduction into human RSV based on known aspects of bovine RSV structure and function, as provided in, e.g., Pastey et al., J. Gen. Virol. 76:193-197 (1993); Pastey et al., Virus Res. 29:195-202 (1993); Zamora et al., J. Gen. Virol. 73:737-741 (1992); Mallipeddi et al., J. Gen. Virol. 74:2001-2004 (1993); Mallipeddi et al., J. Gen. Virol. 73:2441-2444 (1992); and Zamora et al., Virus Res. 24:115-121 (1992), each incorporated herein by reference, and in accordance with the teachings disclosed herein. In other embodiments of the invention, mutations of interest for introduction within chimeric RSV are modeled after a tissue culture-adapted nonpathogenic strain of pneumonia virus of mice (the murine counterpart of human RSV) which lacks a cytoplasmic tail of the G protein (Randhawa et al., Virology 207:240-245 (1995)). Accordingly, in one aspect of the invention the cytoplasmic and/or transmembrane domains of one or more of the human RSV glycoproteins, F, G and SH, are added, deleted, modified, or substituted within an attenuated recombinant RSV using a heterologous counterpart sequence (e.g., a sequence from a cytoplasmic, or transmembrane domain of a F, G, or SH protein of murine pneumonia virus) to achieve a desired attenuation. As another example, a nucleotide sequence at or near the cleavage site of the F protein, or the putative attachment domain of the G protein, can be modified by point mutations, site-specific changes, or by alterations involving entire genes or gene segments to achieve novel effects on viral growth in tissue culture and/or infection and pathogenesis.

In addition to the above described modifications to recombinant vaccine viruses, different or additional modifications in viral clones can be made to facilitate manipulations, such as the insertion of unique restriction sites in various intergenic regions or elsewhere. Nontranslated gene sequences can be removed to increase capacity for inserting foreign sequences.

In another aspect of the invention, compositions (e.g., isolated polynucleotides and vectors comprising a recombinant negative stranded RNA viral genome incorporating a mutation identified in a heterologous virus) are provided for producing an isolated attenuated infectious virus. Using these compositions and methods, infectious viruses and subviral particles are generated from a recombinant genome or antigenome and selected viral proteins, e.g., a nucleocapsid (N) protein, a nucleocapsid phosphoprotein (P), a large (L) polymerase protein, and an RNA polymerase elongation factor in the case of RSV. In related aspects of the invention, compositions and methods are provided for introducing the aforementioned structural and phenotypic changes into a recombinant virus to yield infectious, attenuated vaccine viruses.

Introduction of the foregoing defined mutations into an infectious, recombinant virus are achieved by a variety of well known methods as referenced above. By “infectious clone” is meant cDNA or its product, synthetic or otherwise, which can be transcribed into genomic or antigenomic RNA capable of serving as template to produce an infectious virus or subviral particle. Thus, defined mutations can be introduced by conventional techniques (e.g., site-directed mutagenesis) into a cDNA copy of the genome or antigenome. The use of antigenome or genome cDNA subfragments to assemble a complete antigenome or genome cDNA as described herein has the advantage that each region can be manipulated separately (smaller cDNAs are easier to manipulate than large ones) and then readily assembled into a complete cDNA. Thus, the complete antigenome or genome cDNA, or any subfragment thereof, can be used as template for oligonucleotide-directed mutagenesis. This can be through the intermediate of a single-stranded phagemid form, such as using the Muta-Gene® kit of Bio-Rad Laboratories (Richmond, Calif.) or a method using a double-stranded plasmid directly as template such as the Chameleon mutagenesis kit of Stratagene (La Jolla, Calif.), or by the polymerase chain reaction employing either an oligonucleotide primer or template which contains the mutation(s) of interest. A mutated subfragment can then be assembled into the complete antigenome or genome cDNA. A variety of other mutagenesis techniques are known and available for use in producing the mutations of interest in the antigenome or genome cDNA. Mutations can vary from single nucleotide changes to replacement of large cDNA pieces containing one or more genes or genome regions.

Thus, in one illustrative embodiment mutations are introduced by using the Muta-gene phagemid in vitro mutagenesis kit available from Bio-Rad. cDNA encoding a portion of an RSV genome or antigenome is cloned into the plasmid pTZ18U, and used to transform CJ236 cells (Life Technologies). Phagemid preparations are prepared as recommended by the manufacturer. Oligonucleotides are designed for mutagenesis by introduction of an altered nucleotide at the desired position of the genome or antigenome. The plasmid containing the genetically altered genome or antigenome fragment is then amplified and the mutated piece is then reintroduced into the fall-length genome or antigenome clone.

The invention also provides methods for producing an attenuated infectious recombinant virus incorporating a mutation identified in a heterologous virus from one or more isolated polynucleotides, e.g., one or more cDNAs. A cDNA encoding a subject viral genome or antigenome is constructed for intracellular or in vitro coexpression with the necessary viral proteins to form, for example, infectious RSV. By “antigenome” is meant an isolated positive-sense polynucleotide molecule which serves as the template for the synthesis of progeny viral genome. Preferably a cDNA is constructed which is a template for synthesis of a positive-sense version of the genome, corresponding to the replicative intermediate RNA, or antigenome, so as to minimize the possibility of hybridizing with positive-sense transcripts of the complementing sequences that encode proteins that facilitate generation of a transcribing, replicating nucleocapsid. For purposes of the present invention, the genome or antigenome of the recombinant virus need only contain those genes or portions thereof necessary to render the viral or subviral particles encoded thereby infectious. Further, the genes or portions thereof may be provided by more than one polynucleotide molecule, i.e., a gene may be provided by complementation or the like from a separate nucleotide molecule.

By recombinant virus is meant a complete virus or virus-like subviral particle derived directly or indirectly from a recombinant expression system or propagated from virus or subviral particles produced therefrom. The recombinant expression system will employ a recombinant expression vector which comprises an operably linked transcriptional unit comprising an assembly of at least a genetic element or elements having a regulatory role in viral gene expression, for example, a promoter, a structural or coding sequence which is transcribed into viral RNA, and appropriate transcription initiation and termination sequences.

To produce infectious virus from cDNA-expressed genome or antigenome, the genome or antigenome is expressed according to known methods to (i) produce a nucleocapsid capable of RNA replication, and (ii) render progeny nucleocapsids competent for both RNA replication and transcription. Transcription by the genome nucleocapsid provides the other proteins and initiates a productive infection. Alternatively, additional viral proteins essential for a productive infection can be supplied by coexpression.

A viral antigenome may be constructed for use in the present invention by, e.g., assembling cloned cDNA segments, representing in aggregate the complete antigenome, by polymerase chain reaction (PCR; described in, e.g., U.S. Pat. Nos. 4,683,195 and 4,683,202, and PCR Protocols: A Guide to Methods and Applications, Innis et al., eds., Academic Press, San Diego (1990), incorporated herein by reference) of reverse-transcribed copies of viral mRNA or genome RNA. For example, cDNAs containing the antigenome or portions thereof are assembled in an appropriate expression vector, such as a plasmid or various available cosmid, phage, or DNA virus vectors. The vector may be modified by mutagenesis and/or insertion of synthetic polylinker containing unique restriction sites designed to facilitate assembly. In some cases, such as RSV, use of a particular vector (pBR322) stabilizes the viral sequence which may otherwise sustain nucleotide deletions or insertions. Likewise, propagation of plasmid may be facilitated by selection of a particular bacterial strain (e.g., DH10B) to avoid an artifactual duplication and insertion which otherwise occurs (e.g., in the vicinity of nt 4499 for RSV).

In certain embodiments of the invention, complementing sequences encoding proteins necessary to generate a transcribing, replicating viral nucleocapsid are provided by one or more helper viruses. Such helper viruses can be wild-type or mutant. Preferably, the helper virus can be distinguished phenotypically from the virus encoded by the cDNA. For example, it is desirable to provide monoclonal antibodies which react immunologically with the helper virus but not the virus encoded by the cDNA. Such antibodies can be neutralizing antibodies. In some embodiments, the antibodies can be used in affinity chromatography to separate the helper virus from the recombinant virus. To aid the procurement of such antibodies, mutations can be introduced into the cDNA to provide antigenic diversity from the helper virus, such as in the RSV HN or F glycoprotein genes.

A variety of nucleotide insertions and deletions can be made in the viral genome or antigenome to generate a modified, attenuated viral clone. Members of the Paramyxovirus and Morbillivirus genera typically abide by a “rule of six,” i.e., genomes (or minigenomes) replicate efficiently only when their nucleotide length is a multiple of six (thought to be a requirement for precise spacing of nucleotide residues relative to encapsidating N protein).

Alternative means to construct cDNA encoding a recombinant negative stranded RNA viruses incorporating a mutation identified in a heterologous virus include by reverse transcription-PCR using improved PCR conditions (e.g., as described in Cheng et al., Proc. Natl. Acad. Sci. USA 91:5695-5699 (1994), incorporated herein by reference) to reduce the number of subunit cDNA components to as few as one or two pieces. In other embodiments different promoters can be used (e.g., T3, SP6) or different ribozymes (e.g., that of hepatitis delta virus. Different DNA vectors (e.g., cosmids) can be used for propagation to better accommodate the large size genome or antigenome.

Isolated polynucleotides (e.g., cDNA) encoding the viral genome or antigenome and, separately or in cis, the necessary viral proteins, are inserted by transfection, electroporation, mechanical insertion, transduction or the like, into cells which are capable of supporting a productive viral infection, e.g., HEp-2, FRhL-DBS2, MRC, and Vero cells. Transfection of isolated polynucleotide sequences may be introduced into cultured cells by, for example, calcium phosphate-mediated transfection (Wigler et al., Cell 14:725 (1978); Corsaro and Pearson, Somatic Cell Genetics 7:603 (1981); Graham and Van der Eb, Virology 52:456 (1973)), electroporation (Neumann et al., EMBO J. 1:841-845 (1982)), DEAE-dextran mediated transfection (Ausubel et al., (ed.) Current Protocols in Molecular Biology, John Wiley and Sons, Inc., NY (1987), cationic lipid-mediated transfection (Hawley-Nelson et al., Focus 15:73-79 (1993)) or a commercially available transfection regent, e.g., LipofectACE® (Life Technologies) (each of the foregoing references are incorporated herein by reference).

The viral proteins are encoded by one or more expression vectors which can be the same or separate from that which encodes the genome or antigenome, and various combinations thereof. Additional proteins may be included as desired, encoded by its own vector or by a vector encoding essential viral proteins. Expression of the genome or antigenome and proteins from transfected plasmids can be achieved, for example, by each cDNA being under the control of a promoter for T7 RNA polymerase, which in turn is supplied by infection, transfection or transduction with an expression system for the T7 RNA polymerase, e.g., a vaccinia virus MVA strain recombinant which expresses the T7 RNA polymerase (Wyatt et al., Virology, 210:202-205 (1995), incorporated herein by reference). The viral proteins, and/or T7 RNA polymerase, can also be provided from transformed mammalian cells, or by transfection of preformed mRNA or protein.

Alternatively, synthesis of antigenome or genome can be conducted in vitro (cell-free) in a combined transcription-translation reaction, followed by transfection into cells. Or, antigenome or genome RNA can be synthesized in vitro and transfected into cells expressing viral proteins.

To select candidate vaccine viruses according to the invention, the criteria of viability, attenuation and immunogenicity are determined according to well known methods. Viruses which will be most desired in vaccines of the invention must maintain viability, have a stable attenuation phenotype, exhibit replication in an immunized host (albeit at lower levels), and effectively elicit production of an immune response in a vaccinee sufficient to confer protection against serious disease caused by subsequent infection from wild-type virus. In this context, viruses of the invention are not only viable and more attenuated then previous mutants, but are more stable genetically in vivo than those previously studied mutants—retaining the ability to stimulate a protective immune response and in some instances to expand the protection afforded by multiple modifications, e.g., induce protection against different viral strains or subgroups, or protection by a different immunologic basis, e.g., secretory versus serum immunoglobulins, cellular immunity, and the like.

To propagate recombinant negative stranded RNA viruses incorporating a mutation identified in a heterologous virus, a number of cell lines which allow for viral growth may be used. Preferred cell lines for propagating attenuated viruses for vaccine use include DBS-FRhL-2, MRC-5, and Vero cells. Highest RSV yields are usually achieved with epithelial cell lines such as Vero cells. Cells are typically inoculated with virus at a multiplicity of infection ranging from about 0.001 to 1.0 or more, and are cultivated under conditions permissive for replication of the virus, e.g., at about 30-37° C. and for about 3-5 days, or as long as necessary for virus to reach an adequate titer. Virus is removed from cell culture and separated from cellular components, typically by well known clarification procedures, e.g., centrifugation, and may be further purified as desired using procedures well known to those skilled in the art.

Recombinant virus which has been attenuated as described herein can be tested in various well known and generally accepted in vitro and in vivo models to confirm adequate attenuation, resistance to phenotypic reversion, and immunogenicity for vaccine use. In in vitro assays, the modified virus (e.g., a multiply attenuated, biologically-derived or recombinant virus) is tested for temperature sensitivity of virus replication, i.e. ts phenotype, and for the small plaque phenotype. Modified viruses are further tested in animal models of viral infection. A variety of animal models for RSV have been described and are summarized in Meignier et al., eds., Animal Models of Respiratory Syncytial Virus Infection, Merieux Foundation Publication, (1991), which is incorporated herein by reference. A cotton rat model of RSV infection is described in U.S. Pat. No. 4,800,078 and Prince et al., Virus Res. 3:193-206 (1985), which are incorporated herein by reference, and is considered reasonable predictive of attenuation and efficacy in humans and non-human primates. In addition, a primate model of RSV infection using the chimpanzee is reasonably predictive of attenuation and efficacy in humans, as is described in detail in Richardson et al., J. Med. Virol. 3:91-100 (1978); Wright et al., Infect. Immun. 37:397-400 (1982); Crowe et al., Vaccine 11:1395-1404 (1993), each incorporated herein by reference. Other models are known for a wide range of negative stranded RNA viruses. The correlation of data derived from these animal models relating to the level of attenuation and immunogenicity of recombinant negative stranded RNA viruses is generally accepted. For example, the therapeutic effect of RSV neutralizing antibodies in infected cotton rats has been shown to be highly relevant to subsequent experience with immunotherapy of monkeys and humans infected with RSV. Indeed, the cotton rat appears to be a reasonably reliable experimental surrogate for the response of infected monkeys, chimpanzees and humans to immunotherapy with RSV neutralizing antibodies. For example, the amount of RSV neutralizing antibodies associated with a therapeutic effect in cotton rats as measured by the level of such antibodies in the serum of treated animals (i.e., serum RSV neutralization titer of 1:302 to 1:518) is in the same range as that demonstrated for monkeys (i.e., titer of 1:539) or human infants or small children (i.e., 1:877). A therapeutic effect in cotton rats was manifest by a one hundred fold or greater reduction in virus titer in the lung (Prince et al., J. Virol. 61:1851-1854) while in monkeys a therapeutic effect was observed to be a 50-fold reduction in pulmonary virus titer. (Hemming et al., J. Infect. Dis. 152:1083-1087 (1985)). Finally, a therapeutic effect in infants and young children who were hospitalized for serious RSV bronchiolitis or pneumonia was manifest by a significant increase in oxygenation in the treated group and a significant decrease in amount of RSV recoverable from the upper respiratory tract of treated patients. (Hemming et al., Antimicrob. Agents Chemother. 31:1882-1886 (1987)). Therefore, based on these studies, the cotton rat constitutes a relevant model for predicting success of chimeric and non-chimeric RSV vaccines in infants and small children. Other rodents, including mice, will also be similarly useful because these animals are moderately permissive for RSV replication and have a core temperature more like that of humans (Wright et al., J. Infect. Dis. 122:501-512 (1970) and Anderson et al., J. Gen. Virol. 71:(1990)). Like models are available and widely known for other negative stranded RNA viruses.

In accordance with the foregoing description and based on the examples below, the invention also provides isolated, infectious attenuated viral compositions for vaccine use. The attenuated virus which is a component of a vaccine is in an isolated and typically purified form. By isolated is meant to refer to RSV which is in other than a native environment of a wild-type virus, such as the nasopharynx of an infected individual. More generally, isolated is meant to include the attenuated virus as a component of a cell culture or other artificial medium. For example, attenuated RSV of the invention may be produced by an infected cell culture, separated from the cell culture and added to a stabilizer.

Vaccines of the invention contain as an active ingredient an immunogenically effective amount of a recombinant negative stranded RNA virus bearing a mutation identified in a heterologous virus as described herein. Recombinant virus can be used directly in vaccine formulations, or lyophilized. Lyophilized virus will typically be maintained at about 4° C. When ready for use the lyophilized virus is reconstituted in a stabilizing solution, e.g., saline or comprising SPG, Mg⁺⁺ and HEPES, with or without adjuvant, as further described below. The biologically-derived or recombinantly modified virus may be introduced into a host with a physiologically acceptable carrier and/or adjuvant. Useful carriers are well known in the art, and include, e.g., water, buffered water, 0.4% saline, 0.3% glycine, hyaluronic acid and the like. The resulting aqueous solutions may be packaged for use as is, or lyophilized, the lyophilized preparation being combined with a sterile solution prior to administration, as mentioned above. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like. Acceptable adjuvants include incomplete Freund's adjuvant, aluminum phosphate, aluminum hydroxide, or alum, which are materials well known in the art. Preferred adjuvants also include Stimulon™ QS-21 (Aquila Biopharmaceuticals, Inc., Worchester, Mass.), MPL™ (3-O-deacylated monophosphoryl lipid A; RIBI ImmunoChem Research, Inc., Hamilton, Mont.), and interleukin-12 (Genetics Institute, Cambridge, Mass.).

Upon immunization with an attenuated viral vaccine composition as described herein, via aerosol, droplet, oral, topical or other route, the immune system of the host responds to the vaccine by producing antibodies specific for one or more viral proteins, e.g., RSV F and/or G glycoproteins. As a result of the vaccination, the host becomes at least partially or completely immune to infection by the subject virus, or resistant to developing moderate or severe disease related thereto.

Vaccines of the invention may comprise attenuated virus that elicits an immune response against a single viral strain or antigenic subgroup, e.g. RSV A or B, or against multiple strains or subgroups. In this context, a chimeric vaccine virus can elicit a monospecific immune response or a polyspecific immune response against multiple strains or subgroups. Alternatively, chimeric viruses having different immunogenic characteristics can be combined in a vaccine mixture or administered separately in a coordinated treatment protocol to elicit more effective protection against one RSV strain, or against multiple RSV strains or subgroups.

The host to which the vaccine is administered can be any mammal susceptible to infection by the subject virus or a closely related virus and capable of generating a protective immune response to antigens of the vaccinating virus. Thus, suitable hosts include humans, non-human primates, bovine, equine, swine, ovine, caprine, lagamorph, rodents, etc. Accordingly, the invention provides methods for creating vaccines for a variety of human and veterinary uses.

The vaccine compositions containing the attenuated recombinant virus of the invention are administered to a patient susceptible to or otherwise at risk of infection by the subject virus in an “immunogenically effective dose” which is sufficient to induce or enhance the individual's immune response capabilities against the subject virus. In the case of humans, the attenuated virus of the invention is administered according to well established human vaccine protocols, e.g., as described for RSV in, Wright et al., Infect Immun. 37:397-400 (1982), Kim et al., Pediatrics 52:56-63 (1973), and Wright et al., J. Pediatr. 88:931-936 (1976), which are each incorporated herein by reference. Briefly, adults or children are inoculated intranasally via droplet with an immunogenically effective dose of RSV vaccine, typically in a volume of 0.5 ml of a physiologically acceptable diluent or carrier. This has the advantage of simplicity and safety compared to parenteral immunization with a non-replicating vaccine. It also provides direct stimulation of local respiratory tract immunity, which plays a major role in resistance to RSV. Further, this mode of vaccination effectively bypasses the immunosuppressive effects of RSV-specific maternally-derived serum antibodies, which typically are found in the very young. Also, while the parenteral administration of RSV antigens can sometimes be associated with immunopathologic complications, this has never been observed with a live virus.

In all subjects, the precise amount of attenuated viral vaccine administered and the timing and repetition of administration will be determined based on the patient's state of health and weight, the mode of administration, the nature of the formulation, etc. Dosages will generally range from about 10³ to about 10⁶ plaque forming units (PFU) or more of virus per patient, more commonly from about 10⁴ to 10⁵ PFU virus per patient. In any event, the vaccine formulations should provide a quantity of attenuated virus of the invention sufficient to effectively stimulate or induce an anti-viral immune response, e.g., as can be determined by complement fixation, plaque neutralization, and/or enzyme-linked immunosorbent assay, among other methods. In this regard, individuals are also monitored for signs and symptoms of upper respiratory illness. As with administration to chimpanzees, the attenuated virus of the vaccine grows in the nasopharynx of vaccinees at levels approximately 10-fold or more lower than wild-type virus, or approximately 10-fold or more lower when compared to levels of incompletely attenuated virus.

In neonates and infants, multiple administration may be required to elicit sufficient levels of immunity. For RSV infection, administration should begin within the first month of life, and at intervals throughout childhood, such as at two months, six months, one year and two years, as necessary to maintain sufficient levels of protection against native (wild-type) RSV infection. Similarly, adults who are particularly susceptible to repeated or serious RSV infection, such as, for example, health care workers, day care workers, family members of young children, the elderly, individuals with compromised cardiopulmonary function, may require multiple immunizations to establish and/or maintain protective immune responses. Levels of induced immunity can be monitored by measuring amounts of neutralizing secretory and serum antibodies, and dosages adjusted or vaccinations repeated as necessary to maintain desired levels of protection. Further, different vaccine viruses may be indicated for administration to different recipient groups. For example, an engineered recombinant expressing a cytokine or an additional protein rich in T cell epitopes may be particularly advantageous for adults rather than for infants. Vaccines produced in accordance with the present invention can be combined with viruses expressing antigens of another subgroup or strain of virus to achieve protection against multiple subgroups or strains. Alternatively, the vaccine virus may incorporate protective epitopes of multiple viral strains or subgroups engineered into one clone as described herein.

Typically when different vaccine viruses are used they will be administered in an admixture simultaneously, but they may also be administered separately. For example, as the F glycoproteins of the two RSV subgroups differ by only about 11% in amino acid sequence, this similarity is the basis for a cross-protective immune response as observed in animals immunized with RSV or F antigen and challenged with a heterologous strain. Thus, immunization with one strain may protect against different strains of the same or different subgroup. However, it is likely that it would be preferable to have the G and F glycoproteins of RSV antigenic subgroup A or B present in a vaccine.

Attenuated recombinant vaccines of the invention elicit production of an immune response that is protective against serious disease, such as pneumonia and bronchiolitis, when the individual is subsequently infected with wild-type virus. While the naturally circulating virus may still be capable of causing infection, there is a very greatly reduced possibility of disease symptoms as a result of the vaccination and possible boosting of resistance by subsequent infection by wild-type virus. Following vaccination, there are detectable levels of host engendered serum and secretory antibodies which are capable of neutralizing homologous (of the same subgroup) wild-type virus in vitro and in vivo. In many instances the host antibodies will also neutralize wild-type virus of a different, non-vaccine subgroup.

Preferred attenuated viruses of the present invention exhibit a very substantial diminution of virulence when compared to wild-type virus that is circulating naturally in humans. The chimeric virus is sufficiently attenuated so that symptoms of infection will not occur in most immunized individuals. In some instances the attenuated virus may still be capable of dissemination to unvaccinated individuals. However, its virulence is sufficiently abrogated such that severe lower respiratory tract infections in the vaccinated or incidental host do not occur.

The level of attenuation of vaccine viruses of the invention may be determined by, for example, quantifying the amount of virus present, e.g., in the respiratory tract, in an immunized host and comparing the amount to that produced by wild-type or other attenuated viruses which have been evaluated as candidate vaccine strains. For example, attenuated RSV of the invention will have a greater degree of restriction of replication in the upper respiratory tract of a highly susceptible host, such as a chimpanzee, compared to the levels of replication of wild-type virus, e.g., 10- to 1000-fold less. Also, the level of replication of attenuated RSV vaccine strains in the upper respiratory tract of the chimpanzee should be less than that of the RSV A2 ts-1 mutant, which was demonstrated previously to be incompletely attenuated in seronegative human infants. In order to further reduce the development of rhinorrhea, which is associated with the replication of virus in the upper respiratory tract, an ideal vaccine candidate virus should exhibit a restricted level of replication in both the upper and lower respiratory tract. However, the attenuated viruses of the invention must be sufficiently infectious and immunogenic in humans to confer protection in vaccinated individuals. Methods for determining levels of RSV in the nasopharynx of an infected host are well known in the literature. Specimens are obtained by aspiration or washing out of nasopharyngeal secretions and virus quantified in tissue culture or other by laboratory procedure. See, for example, Belshe et al., J. Med. Virology 1:157-162 (1977), Friedewald et al., J. Amer. Med. Assoc. 204:690-694 (1968); Gharpure et al., J. Virol. 3:414-421 (1969); and Wright et al., Arch. Ges. Virusforsch. 41:238-247 (1973), each incorporated herein by reference. The virus can conveniently be measured in the nasopharynx of host animals, such as chimpanzees. Other methods for determining levels and virulence of additional negative stranded RNA viruses are widely known and readily practiced.

The invention disclosed herein is further clarified by the following examples which are offered by way of illustration not limitation. For the purposes of the present description, all publications and patent documents cited herein are incorporated by reference in their entirety for all purposes.

### Example I

### Mapping Attenuating Mutations Identified in HPIV3 JS cp45 and RSV cp530 and Sendai Virus to Conserved Sequence Elements Among Heterologous Negative Stranded RNA Viruses

The present example demonstrates that mutations identified in one mutant negative stranded RNA virus can be readily mapped to corresponding, conserved amino acid sequence elements in heterologous viruses within the order Mononegavirales. These mutations that are characterized by a structural change compared to a parental sequence, which parental sequence is conserved (identically or conservatively) among one or more heterologous negative stranded RNA viruses, provide likely candidate mutations for incorporation within recombinant viruses sharing the parental protein sequence elements.

To exemplify this aspect of the invention, a panel of known mutations within the HPIV3 mutant strain JS cp45 was analyzed. This panel of mutations includes ts attenuating amino acid substitutions in the polymerase L gene at parental residue/sequence positions Tyr₉₄₂, Leu₉₉₂, and/or Thr₁₅₅₈. More specifically, the JS cp45 mutant L protein exhibits attenuating mutations where the parental Tyr₉₄₂ is replaced by His, Leu₉₉₂ is replaced by Phe, and/or Thr₁₅₅₈ is replaced by Ile (see, U.S. patent application Ser. No. 08/083,793 and corresponding International Application No. WO 98/53078, incorporated herein by reference). In accordance with preferred aspects of the invention, these mutations were not only mapped against parental sequences to identify the changes, but were also successfully incorporated in PIV recombinants (r942, r992, r1558, r942/992, r992/1558, r942/1558, and r942/992/1558), singly and in combination, to confirm their attenuating effects and recoverability into cloned, infectious virus.

Additional exemplary mutations were evaluated in the HPIV3 JS cp45 mutant which were mapped and characterized to encode attenuating amino acid substitutions in the F and C proteins of HPIV3. These mutations include non-ts attenuating amino acid substitutions in the C protein at the parental residue/position Ile₉₆ of JS HPIV3, as exemplified by the substitution of Ile₉₆ to Thr. Further exemplary mutations identified in the F protein of HPIV3 encode amino acid substitutions at parental residue/positions Ile₄₂₀ and Ala₄₅₀, as exemplified by the substitutions Ile₄₂₀ to Val and Ala₄₅₀ to Thr.

Each attenuating mutation thus identified in HPIV3 provided an index for sequence comparison against a homologous protein in other negative stranded viruses. To illustrate this aspect of the invention, conventional sequence alignments were undertaken in accordance with the methods outlined above to map the mutations identified in the HPIV3 L and F proteins to corresponding sites among a panel of heterologous negative stranded RNA viruses (HPIV1, Sendai virus, HPIV2, BPIV3, MeV and RSV). As shown in Table 2, this exercise revealed, quite remarkably, a high degree of conservation between parental sequence elements of the selected mutants and the wild-type sequences of the heterologous viruses to which they were compared. Such results were surprising, because evolutionarily conserved sequence elements are imputed to have important functional significance that would be predicted to yield more severe phenotypic effects, eg., loss of viability or infectivity, upon mutation than mere attenuation.

TABLE 2

Sequence alignment of the region around the attenuating mutations identified in
the F and L proteins HPIV3 cp45

HPIV3 cp45 F ORF 1420V and A450T

| | | | |
|---|---|---|---|
| | | * * | |
| HPIV3 | 407¹ | QGVKIITHKECSTIGINGMLENTNKEGTLAFYTPNDITLNNSVALDPIDISIE | (SEQ ID NO. 1) |
| BPIV3 | 407 | QGIKIITHKECQVIGINGMLFNTNREGTLATYTFDDIILNNSVALNPIDISME | (SEQ ID NO. 2) |
| HPIV1 | 410 | RGVTFLTYTNCGLIGINGIELYANKRGRDTTWGNQIIKVGPAVSIRPVDISLN | (SEQ ID NO. 3) |
| HPIV2 | 404 | QGISIIDIKRCSEMMLDTFSFRITSTFNATYVTDFSMINANIVHLSPLDLSNQ | (SEQ ID NO. 4) |
| RSV | 437 | NGCDYVSNKGVDTVSVGNTLYYVNKQEGKSLYVKGEPIINFYDPLVFPSDEFD | (SEQ ID NO. 7) |
| MEASLES | 410 | KILTYIAADHCPVVEVNGVTIQVGSRRYPDAVYLHRIDLGPPISLERLDVGTN | (SEQ ID NO. 8) |

HPIV3 cp45 L ORF Y942H

| | | | |
|---|---|---|---|
| | | * | |
| HPIV3 | 923 | NPNWMQYASL...IPASVGGFNYMAMSRCFVRNIGDPSVAALAD | (SEQ ID NO. 9) |
| BPIV3 | 923 | NIHWMQYASL...IPASVGGFNYMAMSRCFVRNIGDPTVAALAD | (SEQ ID NO. 10) |
| SENDAI | 923 | GKNWLRCAVL...IPANVGGFNYMSTSRCFVRNIGDPAVAALAD | (SEQ ID NO. 11) |
| HPIV2 | 927 | HPRLISRIVL...LPSQLGGLNYLACSRLFNRNIGDPLGTAVAD | (SEQ ID NO. 12) |
| MEASLES | 923 | NNDLLIRMAL...LPAPIGGMNYLNMSRLFVRNIGDPVTSSIAD | (SEQ ID NO. 13) |
| RSV | 968 | LDNIDTALTLYMNLPMLFGGGDPNLLYRSFYRRTPDFLTEAIVH | (SEQ ID NO. 14) |

HPIV3 cp45 L ORF L992F

| | | | |
|---|---|---|---|
| | | * | |
| HPIV3 | 973 | LDRSVLYRIMNQEPGESSFLDWASDPYSCNLPQSQNITTMIKNITA | (SEQ ID NO. 15) |
| BPIV3 | 973 | LDRGVLYRIMNQEPGESSFLDWASDPYSCNLPQSQNITTMIKNITA | (SEQ ID NO. 16) |
| SENDAI | 973 | LDKQVLYRVMNQEPGDSSFLDWASDPYSCNLPHSQSITTIIKNITA | (SEQ ID NO. 17) |
| HPIV2 | 977 | LESWILYNLLARKPGKGSWATLAADPYSLNQEYLYPPTTILKRHTQ | (SEQ ID NO. 18) |
| MEASLES | 973 | MPEETLHQVMTQQPGDSSFLDWASDPYSANLVCVQSITRLLKNITA | (SEQ ID NO. 19) |
| RSV | 1036 | LNKFLTCIITFDKNPNAEFVTLMRDPQALGSERQAKITSEINRLAV | (SEQ ID NO. 20) |

HPIV3 cp45 L ORF T1558I

| | | | |
|---|---|---|---|
| | | * | |
| HPIV3 | 1537 | HPKVFKRFWDCGVLNPIYDPNTASQDQIKLALSICEYSLDLFMREWL | (SEQ ID NO. 21) |
| BPIV3 | 1537 | HPKVFKRFWDCGVLNPIYGPNTASQDQVKLALSICEYSLDLFMREWL | (SEQ ID NO. 22) |
| SENDAI | 1537 | HPKIFKRFWNAGVVEPVYGPNLSNQDKILLALSVCEYSVDLFMHDWQ | (SEQ ID NO. 23) |
| HPIV2 | 1543 | HPKLLRRAMNLDIITPIHAPYLASLDYVKLSIDAIQWGVKQVLADLS | (SEQ ID NO. 24) |
| MEASLES | 1535 | HPKIYKKFWHCGIIEPIHGPSLDAQNLHTTVCNMVYTCYMTYLDLLL | (SEQ ID NO. 25) |
| RSV | 1584 | EQKVIKYILSQDASLHRVEGCHSFKLWFLKRLNVAEFTVCPWVVNID | (SEQ ID NO. 26) |

*Indicates mutated amino acid in HPIV3 cp45. Underlining indicates amino acid residues shared between different viruses.
¹Position of the first amino acid of the sequence shown in the full length protein.

Reviewing the exemplary alignment shown in Table 2, it is notable that each of the L and F mutations analyzed changed a parental sequence element that was substantially conserved, marked either by the presence of identical or conservative amino acid residues, at corresponding positions among the viral taxa shown. For example, the cp45 L protein mutation changing the parental residue Tyr₉₄₂ to His mapped highly conservatively, as indicated by retention of identical tyrosine residues at corresponding wild-type positions in each of the Sendai, HPIV2, HPIV3, BPIV3 and MeV L proteins (Table 2). Similarly, the cp45 L mutation featuring Leu₉₉₂ replaced by Phe maps to a parental residue/position that is identically conserved between the Sendai, HPIV3, BPIV3 and MeV L proteins. The two identified mutations in the cp45 F protein, substituting Ile₄₂₀ to Val and Ala₄₅₀ to Thr, also exhibit identically conserved residue/positions between HPIV3 and BPIV3, while the Ile₄₂₀ to Val mutant parental residue is further identically conserved in HPIV1. Additional conserved sequence elements corresponding to sites of attenuating mutations identified in HPIV3 JS cp45 are also shown in Table 2.

Another heterologous sequence alignment was conducted to evaluate conservation of the exemplary RSV attenuating mutation identified in the mutant strain RSV cp530. This mutation is marked by a substitution of phenylalanine to leucine at position 521 in the L polymerase of cp530, which mutation occurs within a larger conserved structural domain of the protein. As is shown in Table 3, the mutation at Phe₅₂₁ also mapped highly conservatively, as indicated by retention of identical phenylalanine residues at corresponding wild-type positions in each of thirteen subject taxa (Table 3; FIG. 1, panel A).

TABLE 3

Alignment of L Polymerase Sequence Around PIV3 PHE-456(*) In Various Paramyxovirus Virus Species

| | | | |
|---|---|---|---|
| | Position of | Position of | Gene bank |
| | First aa in | conserved | Accession |
| Virus | sequence | phenylanine | No. |

| | | | | | |
|---|---|---|---|---|---|
| PIV3 | 431 | NAYGSNSAISYENAVDYYQSFIGIKFNKFIEPQLDEDLTIY | (SEQ ID NO. 27) | 456 | Z11575 |
| RSV | 496 | YYKLNTYPSLLELTERDLIVLSGLRFYREFRLPKKVDLEMI | (SEQ ID NO. 28) | 521 | P28887 |
| MEASLES | 423 | NAQASGEGLTHEQCVDNWKSFAGVKFGCFMPLSLDSDLTMY | (SEQ ID NO. 29) | 448 | P35975 |
| SENDAI | 431 | NAQGSNTAISYECAVDNYTSFIGFKFRKFIEPQLDEDLTIY | (SEQ ID NO. 30) | 456 | Q06996 |
| PIV2 | 434 | EFQHDNAEISYEYTLKHWKEISLIEFRKCFDFDPGEELSIF | (SEQ ID NO. 31) | 459 | P26676 |
| CDV | 423 | NAHASGEGITYSQCIENWKSFAGIRFKCFMPLSLDSDLTMY | (SEQ ID NO. 32) | 448 | P24658 |
| SV41 | 435 | ELHHDNSEISYEYTLRHWKELSLIEFKKCFDFDPGEELSIF | (SEQ ID NO. 33) | 460 | P35341 |
| PDV | 423 | NACVSGEGITYSQCVENWKSFAGIKFRCFMPLSLDSDLTMY | (SEQ ID NO. 34) | 448 | Y09630 |
| HENDRA | 430 | RLKNSGESLTVDDCVKNWESFCGIQFDCPMELKLDSDLSMY | (SEQ ID NO. 35) | 455 | AF017149 |
| SV5 | 433 | ELMNDNTEISYEFTLKHWKEVSLIKFKKCFDADAGEELSIF | (SEQ ID NO. 36) | 458 | Q88434 |
| RINDERPEST | 423 | NAQASGEGLTYEQCVDNWKSFAGIRFGCFMPLSLDSDLTMY | (SEQ ID NO. 37) | 448 | P41357 |
| APV | 431 | YMNAKTYPSNLELCVEDFLELAGISFCQEFYVPSQTSLEMV | (SEQ ID NO. 38) | 456 | U65312 |
| NDV | 427 | QLHADSAEISHDIMLREYKSLSALEFEPCIEyDpVTNLSMF | (SEQ ID NO. 39) | 452 | X05399 |

Amino acids in bold type are conserved in all virus species analyzed

Yet another heterologous sequence alignment was conducted to demonstrate the ability to identify conserved structural elements corresponding to sites of known attenuating mutations. In this example, an attenuating mutation identified at position 170 in the C protein of Sendai virus was mapped against corresponding sequences in the C proteins of the heterologous viruses HPIV-1, HPIV-3 and BPIV-3 (see, Table 4; positions of first residues in corresponding sequences are numbered). Once again, this mutation was marked by an amino acid change at a parental residue/sequence position that was identically conserved among diverse taxa.

TABLE 4

| | | | |
|---|---|---|---|
| | | 164 | |
| HPIV3 | 144 | MKLERWIRTLLRGKCDNLQMFQARYQEVMTYLQQNKVETVIMEEAWNLSVHLIQDQ* | (SEQ ID NO. 40) |
| BPIV3 | 144 | MKLERWIRTLLRGKCDNLKMFQSRYQEVMPFLQQNKMETVMMEEAWNLSVHLIQDIPA* | (SEQ ID NO. 41) |
| SeV | 150 | MKTERWLRTLIRGEKTKLKDFQKRYEEVEPYLMKEKVEQIIMEEAWSLAAHIVQE* | (SEQ ID NO. 42) |
| HPIV-1 | 150 | MKTERWLRTLIRGKKTKLRDFQKRYEEVHPYLMMERVEQIIMEEAWKLAAHIVQE* | (SEQ ID NO. 43) |
| | | 170 | |

These collective findings illustrate surprising conservation of sequence elements that correspond to sites of attenuating mutations. On this basis, the rational design methods of the invention were initiated to import attenuating sequence changes identified in one heterologous virus to a different recombinant virus, eg., by identical or conservative alteration of a recombinant genome or antigenome, to yield an attenuated, recombinant clone. Practical development of these methods is directly evinced by the following examples.

### Example II

### Heterologous Transfer of an Attenuating Mutation from RSV cpts530 L into a Recombinant HPIV3 Vaccine Candidate

Having thus identified attenuating mutations at sites that correspond to conserved structural elements between heterologous taxa within the Mononegavirales, the concept of heterologous transfer of attenuating mutations across phylogenetic boundaries was tested using the important disease virus RSV as a model. In this context, the present example demonstrates that an attenuating (att) mutation identified in RSV, a virus in the pneumovirus genus of the Paramyxoviridae family, can be transferred to PIV3, a member of the distantly-related Respirovirus genus. These viruses represent two different subfamilies within the Paramyxoviridae Family, Pneumovirinae and Paramyxovirinae, respectively.

More specifically, an attenuating mutation in a first, heterologous virus (RSV cpts530), which altered the parental RSV sequence at a defined site of mutation (Phe₅₂₁ of the RSV L protein) to specify the attenuated phenotype mapped to an identically conserved residue at the corresponding position (F456L) in the L protein of a selected target virus, HPIV3 (Table 3). The wild-type HPIV3 sequence element thus conserved was therefore tested as a potential target for heterologous transfer of the attenuating mutation between RSV and HPIV3.

To achieve this transfer, all or part of the conserved sequence element(s) bearing the mutant alteration is preferably copied or imported into the recombinant virus to yield a novel attenuated derivative. While it is preferred to identically copy the mutant alteration into the recombinant virus, this level of fidelity is not required. On the contrary, the parental or wild-type residue thus identified as a target may be deleted or replaced by an amino acid insertion at the site of mutation comprising one or more residues that may be unrelated to the residue(s) marking the mutation. Preferably, where the subject mutation is marked by one or more amino acid substitution(s), the residue(s) in the parent clone of the recombinant virus corresponding in position to the site of the mutation is/are replaced by one or more residues that are conservatively related to the substitute residue(s) identified in the mutant sequence. More preferably, the residue(s) in the parent clone of the recombinant virus corresponding to the site of the mutation is/are replaced by one or more identical residue(s) to those present in the mutant sequence.

Thus, in the present example, the phenylalanine to leucine mutation at aa position 521 in the L polymerase of cpts530 was characterized to specify ts and attenuation (att) phenotypes. Sequence alignment of this region in the L proteins of several distantly-related paramyxoviruses (Table 3) revealed that this phenylalanine is broadly conserved. Using reverse genetics, the analogous phenylalanine at position 456 in the L protein of wild type PIV3 was mutagenized to leucine (F456L). The resulting virus, designated r456_{L}, was ts (40° C. shut-off temperature of plaque formation), and its replication in the upper, but not the lower, respiratory tract of hamsters was 10-fold reduced compared to that of the recombinant wild type (rwt) PIV3. These results indicate that the transferred, phenylalanine to leucine mutation specified a similar level of temperature sensitivity and attenuation in two distantly-related paramyxoviruses.

Also within the present example, it is demonstrated that introduction of the F456L mutation into two rPIV3 candidate vaccine viruses, one bearing three cp45 ts missense mutations in the L protein (rcp45_{L}) and the other bearing all 15 cp45 mutations (rcp45) further attenuated the viruses in vivo. More specifically, the F456L mutation was introduced into two recently-developed recombinant PIV3 vaccine candidates. One is rcp45, a recombinant version of the biologically-derived PIV3 vaccine candidate cp45, the other is rcp45_{L}, a version in which the only cp45 mutations present are the three amino acid substitutions in the L protein (published International Application No. 98/53078; Skiadopoulos et al., J. Virol. 72(3):1762-8 (1998); Skiadopoulos et al., J. Virol. 73(2):1374-1381, (1999), each incorporated herein by reference). The addition of the F456L mutation to rcp45 or rcp45_{L} increased its level of temperature sensitivity and attenuation in vivo, and rcp45-456 was immunogenic and phenotypically stable. The rcp45L-456 and rcp45-456 viruses were 100 to 1000-fold more restricted in replication in hamsters than their rcp45_{L} and rcp45 parents. Immunization with rcp45-456 induced a moderate level of resistance to replication of PIV3 challenge virus. In chimpanzees, rcp45-456 was 5-fold more restricted in the respiratory tract than rcp45, and induced a comparable, moderate-to-high level of PIV3-specific serum antibodies. rcp45 and rcp45-456 viruses isolated from chimpanzees throughout a two week course of replication maintained the level of temperature sensitivity of their respective input viruses, illustrating their phenotypic stability.

Therefore, the transfer of the F456L mutation corresponding to the RSVF521L mutation into cp45 recombinant virus resulted in an incremental increase in attenuation of the recombinant virus, demonstrating the usefulness of this transferred mutation for fine tuning attenuation in PIV vaccine candidates. The ability to transfer mutations identified in heterologous paramyxoviruses, which in this case represent different subfamilies, greatly enhances the ability to develop novel parainfluenza virus vaccines.

### Viruses and Cells

The rPIV3 JS wt (referred to as rwt herein), rcp45_{L}, and rcp45 viruses that are used as controls in this example were generated previously (published International Application No. 98/53078; Durbin et al., Virology 235:323-332 (1997); Skiadopoulos et al., J. Virol. 72(3):1762-8 (1998); Skiadopoulos et al., J. Virol. 73(2):1374-1381 (1999), each incorporated herein by reference). The rPIV3s were grown in simian LLC-MK2 cells (ATCC CCL 7.1) as described previously (Durbin et al., Virology 235:323-332, 1997; Hall et al., Virus Res. 22(3):173-184, 1992; Skiadopoulos et al., J. Virol. 72(3): 1762-8 (1998), incorporated herein by reference). The modified vaccinia virus Ankara (MVA-T7) (Wyatt et al., Virology 210(1):202-205 (1995)), which expresses the T7 polymerase, was kindly provided by Linda Wyatt and Bernard Moss. HEp-2 (ATCC CCL 23) and LLC-MK2 cells were maintained in OptiMEM I (Life Technologies, Gaithersburg, Md.) supplemented with 2% FBS and gentamicin sulfate (50 ug/mL) or in EMEM supplemented with 10% FBS, gentamicin sulfate (50 ug/ml), and 4 mM glutamine.

### Construction of Point Mutations in rwt

A subgenomic fragment of p3/7(131)2G+, the antigenomic cDNA clone of PIV3 JS wt previously used to recover infectious virus (Durbin et al., Virology 235:323-332, 1997; Skiadopoulos et al., J. Virol. 72(3):1762-8, 1998; Skiadopoulos et al., J. Virol. 73(2):1374-1381, 1999), encompassing PIV3 nt 7437 to 11312 (Xho I-Sph I), was cloned into a pUC19 vector modified to accept this fragment using standard molecular cloning techniques. The cDNA was modified by the introduction of two point mutations using the Transformer Mutagenesis kit (Clontech, CA) as described previously (Skiadopoulos et al., J. Virol. 72(3):1762-8, 1998; Skiadopoulos et al., J. Virol. 73(2):1374-1381, 1999). These two changes were at positions 10011 (T to C) and 10013 (C to G) numbered according to the complete positive sense sequence of rPIV3 antigenomic RNA. This introduced the F456L codon change as well as an overlapping, silent XmnI site as a marker (FIG. 1). After mutagenesis, restriction endonuclease fragments were sequenced completely and were cloned directly into the full-length clone, p3/7(131)2G+, or into derivatives bearing cp45 mutations, using standard molecular cloning techniques.

### Recovery of Recombinant PIV3s Bearing the F456L Mutation.

Full-length antigenomic cDNA derivatives bearing the F456L mutation and the three support plasmids pTM(N), pTM(P no C) and pTM(L) (Durbin et al., Virology 235:323-332, 1997) were transfected into HEp-2 monolayers in 6-well plates (Costar, Mass.) using LipofectACE (Life Technologies, MD), and the monolayers were infected with MVA-T7 as described previously (Durbin et al., Virology 235:323-332 (1997); Skiadopoulos et al., J. Virol. 72(3):1762-8 (1998). Plasmid pTM(P no C) is a version of the previously-described pTM(P) (Durbin et al., Virology 235:323-332 (1997)) plasmid in which the C ORF has been modified such that its translational initiation codon was changed from AUG to ACG (methionine to threonine). After incubation at 32° C. for 4 days, the transfection harvest was passaged onto LLC-MK2 cells in T-25 flasks which were incubated at 32° C. for four to eight days. The clarified medium supernatant was subjected to three rounds of plaque purification on LLC-MK2 cells as described previously (Durbin et al., Virology 235:323-332 (1997); Hall et al., Virus Res. 22(3):173-184 (1992); Skiadopoulos et al., J. Virol. 72(3):1762-8 (1998)). Each biologically-cloned recombinant virus was amplified twice in LLC-MK2 cells at 32° C. to produce virus for further characterization. Virus was concentrated from clarified medium by polyethylene glycol precipitation (Mbiguino and Menezes, J. Virol. Methods 31:161-170 (1991)), and viral RNA (vRNA) was extracted with TRIzol Reagent (Life Technologies). Reverse transcription was performed on vRNA using the Superscript II Preamplification System (Life Technologies) with random hexamer primers. The Advantage cDNA PCR kit (Clontech, CA) and sense and antisense primers specific for various portions of the PIV3 genome were used to amplify fragments for restriction endonuclease digestion and/or sequence analysis. The PCR fragments were analyzed by sequencing and/or restriction enzyme analysis with each of the restriction enzymes whose recognition sites had been created or ablated during construction of the mutations.

Efficiency of Plaque Formation (EOP) of rPIV3s Bearing the F456L Mutation at Permissive and Restrictive Temperatures

The level of temperature sensitivity of plaque formation in vitro of control and recombinant viruses was determined at 32° C. and at a range of temperatures from 35° C. to 41° C. in LLC-MK2 cell monolayer cultures incubated for six days as previously described (Hall et al., Virus Res. 22(3):173-184, 1992). Plaques were enumerated by hemadsorption with guinea pig erythrocytes following removal of the methylcellulose overlay, or alternatively the viral plaques present in the monolayer were identified by immunoperoxidase staining with a mixture of two PIV3-specific anti-HN murine mAbs 101/1 and 454/11 diluted 1:250 (Murphy et al., Vaccine 8(5):497-502 (1990); Murphy et al. (1990); van Wyke Coelingh, Winter, and Murphy Virology 143(2):569-582, (1985), each incorporated herein by reference).

Evaluation of rPIV3 Mutant Viruses for the att Phenotype in Hamsters

4 week-old Golden Syrian hamsters (Charles River Laboratories, NY) which were seronegative for PIV3 were inoculated intranasally with 0.1 ml L15 medium containing 10^{6.0} TCID₅₀ of rwt or one of the mutant rPIV3s. On day 4 post-infection, the hamsters were sacrificed, the lungs and nasal turbinates were harvested, and the virus was quantified as previously described (Durbin et al., Virology 235:323-332, 1997; Skiadopoulos et al., J. Virol. 72(3):1762-8, 1998). The mean log₁₀ TCID₅₀/gram at 32° C. was calculated for each group of hamsters.

Immunogenicity and Efficacy of rcp45-456 in Hamsters

Three groups of five hamsters were inoculated intranasally with 0.1 ml of: (i) L15 medium (placebo), (ii) 10^{6.0} TCID₅₀ of rcp45, or (iii) 10^{6.0} TCID₅₀ of rcp45-456. The hamsters were bled before infection and 42 days after infection, and serum hemagglutination-inhibiting (HAI) antibody titers against PIV3 were determined as described previously (van Wyke Coelingh, Winter, and Murphy, 1985). On day 44, the hamsters were challenged by intranasal administration of 10^{6.0} TCID₅₀ rwt. Nasal turbinates and lungs were harvested four days later, and the titer of rwt in the tissue homogenates was determined as described above.

Evaluation of rPIV3 Mutant Viruses for the att Phenotype in Chimpanzees

Young male and female chimpanzees, which were seronegative for RSV and PIV3 and weighed 6.6 to 10.0 kg, were pair-housed in large glass isolator suites and maintained as described previously (Crowe et al., 1994a; Hall et al., J. Infect. Dis. 167:958-962 (1993)). Groups of chimpanzees were infected by the intranasal (IN) and intratracheal (IT) routes with a 1 ml inoculum containing 10^{6.0} TCID₅₀ of rcp45 or rcp45-456 at each site. Following inoculation with the virus, nasopharyngeal swab and tracheal lavage samples were collected for quantification of virus shedding as described previously (Hall et al., 1993; Karron et al., 1997). Nasopharyngeal swab samples were collected on days 1 through 10 and on day 13, and tracheal lavage samples were collected on days 2, 4, 6, 8 and 10. The extent of rhinorrhea was estimated daily and assigned a score of 0-4 (0=no rhinorrhea; 1=trace; 2=mild; 3=moderate; 4=severe). The rcp45 and rcp45-456 viruses were evaluated in two separate experiments which followed the same protocol. In experiment 1, two chimpanzees were inoculated with rcp45 and four with rcp45-456, and in experiment 2 each virus was given to 2 animals. The second experiment was performed to confirm the growth difference between the two viruses observed in the first experiment. The two sets of data were indistinguishable with regard to virus growth and immunogenicity and were averaged together. Data from four animals that received 1 TCID₅₀ of the wt JS strain of PIV3 by the IN and IT route were described previously (Hall et al., 1993) and are presented here for the purposes of comparison.

Characterization of Replication of rPIVs in Chimpanzees

The quantity of virus in nasopharyngeal swab and tracheal lavage samples was determined on LLC-MK2 monolayers at 32° C. and expressed as log₁₀ TCID₅₀/ml, as described previously (Crowe et al., 1994a; Hall et al., 1993). Virus present in chimpanzee nasopharyngeal and tracheal lavage samples was grown on LLC-MK2 monolayers in 24 well plates at 32° C. until extensive cytopathic effect was detected. The level of temperature sensitivity of these rPIV3 isolates was estimated by determining the efficiency of plaque formation on LLC-MK2 monolayers as described above. All studies with chimpanzee specimens or isolates included a cocktail of antibiotics consisting of clindamycin (10 ug/ml), ciprofloxacin (100 ug/ml), gentamicin (100 ug/ml), and amphotericin B (2.5 ug/ml).

Results

Introduction of F456L Mutation into a rwt Confers the ts Phenotype

As noted above, RSV cpts530 is ts and attenuated for replication in the respiratory tract of mice (Crowe et al., 1994b). A single amino acid substitution at phenylalanine-521 in the L protein of RSV cpts530 is responsible for the temperature sensitivity (39° C. shut-off temperature of plaque formation) and attenuation (10-fold reduction in replication in the upper respiratory tract of mice) (Crowe et al., 1994b; Juhasz et al., 1997). Sequence alignment of the L proteins of 13 different paramyxoviruses, including RSV and PIV3, revealed that the phenylalanine at position 521 in RSV L is highly conserved (Table 3; FIG. 1, panel A). In the case of HPIV3, the corresponding amino acid occurs at position 456 in the PIV3 L polymerase. The difference in the position number for this residue in RSV (521) versus PIV3 (456) is consistent with previous observations that the L protein of RSV has an amino-terminal extension of about 70 amino acids compared with that of paramyxoviruses from other genera (Stec et al., Virology 183:273-287 (1991)). To determine whether a mutation at the 456 position in the L protein of PIV3 would confer ts and att phenotypes similar to those of the heterologous, RSV cpts530 mutant, the phenylalanine at position 456 in rwt was mutagenized identically to leucine (F456L). The coding change was designed to involve two nucleotide substitutions in order to reduce the probability of reversion to wt. Also, the mutation was marked by introduction of a silent Xmn I restriction endonuclease recognition site (FIG. 1, panel C). The mutations were introduced into the full-length infectious rwt cDNA plasmid, and recombinant virus was recovered as described previously (Durbin et al., Virology 235:323-332 (1997), Skiadopoulos et al., J. Virol. 72(3):1762-8 (1998) and Skiadopoulos et al., J. Virol. 73(2)1374-1381 (1999)). The recovered r456_{L} mutant (FIG. 1, panel B) was confirmed to possess the Xmn I marker and the F456L mutation based on RT-PCR of purified vRNA and analysis by restriction enzyme digestion and sequencing. Introduction of the F456L mutation into rwt conferred a shut-off temperature of 40° C. (Table 5), one degree higher than that of RSV cpts530, demonstrating that a ts mutation identified in a pneumovirus can be transferred to a distantly-related Respirovirus to confer a similar, although not identical, ts phenotype.

TABLE 5
Temperature sensitivity of control viruses and viruses bearing the F456L
mutation in LLC-MK2 cells at permissive and non-permissive temperatures.

| | Mean Titer at | Mean log₁₀pfu/ml reduction at the indicated temperature^{a} | | | | | | |
|---|---|---|---|---|---|---|---|---|
| Virus | 32° C. | 35° C. | 36° C. | 37° C. | 38° C. | 39° C. | 40° C. | 41° C. |
| rwt | 7.5 | −0.2 | 0.0 | 0.0 | −0.1 | 0.1 | 0.3 | 0.8 |
| r456_{L} | 7.3 | — | — | 0.3 | 0.2 | 0.6 | 2.5 | ≧5.5 |
| rcp45_{L} | 7.3 | — | — | 0.5 | 1.9 | 4.3 | ≧6.0 | — |
| rcp45_{L}-456 | 6.5 | 2.5 | ≧4.5 | ≧5.2 | ≧5.2 | ≧5.2 | ≧5.2 | — |
| rcp45 | 7.8 | 0.6 | 1.0 | 1.4 | 2.4 | ≧5.6 | ≧6.8 | — |
| rcp45-456 | 7.7 | 0.2 | 1.0 | 2.2 | ≧4.9 | ≧7.3 | ≧6.9 | — |

^{a}Plaques were enumerated by immunoperoxidase staining after incubation for 6 days at the indicated temperature. The average of three or more experiments is presented. Values which are underlined and in bold type represent the lowest restrictive temperature at which there was a 100-fold reduction of plaquing efficiency, which is defined as the shut-off temperature of plaque formation. The reduction in titer was determined by subtracting the titer at the indicated temperature from that at permissive temperature (32° C.).

Introduction of the F456L Mutation into Recombinant rcp45 Based Attenuated Viruses Increases Temperature Sensitivity

cp45 possesses three specific amino acid substitutions in the L protein, which were shown previously to be major determinants of its ts and att phenotypes (Skiadopoulos et al., J. Virol. 72(3):1762-8 (1998). cp45 also contains 12 other mutations outside L which include ts and att mutations (Skiadopoulos et al., J. Virol. 73(2): 1374-1381 (1999). The F456L mutation was introduced into rcp45 and also into a virus bearing only the three rcp45 L gene mutations (rcp45_{L}). This was done in order to determine if the temperature sensitivity specified by the F456L mutation would be additive with that specified by the three cp45 L mutations or by the full set of 15 cp45 mutations. Remarkably, rcp45_{L}-456 manifested a greatly enhanced level of temperature sensitivity (Table 5) with a shut-off temperature of 35° C. compared with 39° C. for rcp45_{L}. The rcp45-456 mutant had an intermediate shut-off temperature of 37° C., compared with 38° C. for rcp45. Although the temperature sensitivity specified by the F456L mutation is additive to that specified by the mutations in rcp45_{L} and rcp45, the magnitude of the effect was clearly different for the two viruses. Thus, the rcp45-456 containing a greater number of mutations was unexpectedly less ts than rcp45_{L}-456. Evidence of complex interactions amongst cp45 ts mutations has been previously observed for other rPIV3 viruses containing various combinations of cp45 mutations, although the basis for these effects is not understood (Skiadopoulos et al., J. Virol. 72(3):1762-8 (1998) and Skiadopoulos et al., J. Virol. 73(2):1374-1381 (1999).

Replication and Immunogenicity of Mutant rPIV3s in Hamsters.

Hamsters were inoculated IN with 10^{6.0} TCID₅₀ of rwt or with one of several mutant rPIV3's, including virus bearing the F456L mutation alone or in combination with cp45 specific mutations (Table 6). After 4 days, lungs and nasal turbinates were harvested and the level of replication of each virus was determined. The F456L mutation alone had a moderate effect on replication of r456_{L}, resulting in approximately a 10-fold reduction in the nasal turbinates and no apparent restriction of replication in the lungs. This level of attenuation is similar to that of the RSV cpts530 mutant in the upper respiratory tract of mice (Crowe et al., Vaccine 12(8), 691-699 (1994a)). Surprisingly, the addition of the F456L mutation to either rcp45_{L} or rcp45 resulted in recombinants whose replication was significantly more restricted in the upper respiratory tract. This indicates that the F456L mutation can greatly enhance the attenuation of replication of both of these mutant viruses in the upper respiratory tract of hamsters.

TABLE 6
Replication in the respiratory tract of hamsters of rwt and rcp45
derivatives containing the F456L mutation^{a}

| | |
|---|---|
| | Mean virus titer |
| | (log₁₀TCID₅₀/g ± S.E.^{b} |
| Number of | in indicated tissue) |

| Virus | Hamsters | Nasal turbinates | Lungs |
|---|---|---|---|
| rwt | 10 | 6.9 ± 0.1 | 6.3 ± 0.4 |
| r456_{L} | 10 | 6.0 ± 0.1 | 5.7 ± 0.5 |
| rcp45_{L} | 10 | 3.8 ± 0.2 | 1.9 ± 0.2 |
| rcp45_{L}-456 | 5 | ≦1.5 ± 0 | ≦1.5 ± 0 |
| rcp45 | 10 | 4.9 ± 0.2 | 2.1 ± 0.2 |
| rcp45-456 | 5 | ≦1.5 ± 0 | 1.6 ± 0.1 |

^{a}Hamsters were administered 10^{6.0} TCID₅₀ of virus intranasally in a 0.1 mL inoculum. Lungs and nasal turbinates were harvested four days later and virus titer was determined at 32° C. Mean of two experiments.
^{b}S.E: Standard error.

To determine whether the rcp45-456 transfer mutant elicits an immune response, seronegative hamsters were administered 10^{6.0} TCID₅₀ IN of rcp45-456, rcp45, or L15 medium. After 42 days, serum samples were collected, and the animals were challenged on day 44 with rwt. Immunization with rcp45-456 or rcp45 elicited moderate to high titers of serum HAI antibodies, respectively, and induced resistance to replication of rwt challenge virus (Table 7). The level of antibodies and resistance induced by infection with rcp45-456 was less than those induced by rcp45. The lower level of the immune response and protection conferred by rcp45-456 likely reflects its significantly lower level of replication in the respiratory tract of the hamsters.

TABLE 7
Infection of hamsters with rcp45 and rcp45-456 induced resistance
to challenge with rwt^{a}

| | Mean challenge rwt titer^{b} | | |
|---|---|---|---|
| | (log¹⁰TCID₅₀/g ± S.E.) in: | | |
| Virus | Nasal turbinates | Lungs | HAI titer^{c} log2 ± S.E) |
| L15 control | 5.6 ± 0.1 | 4.8 ± 0.2 | <2.0 ± 0 |
| rcp45 | <1.5 ± 0 | <1.5 ± 0 | 10.4 ± 0.2 |
| rcp45-456 | 3.1 ± 0.6 | 2.1 ± 0.6 | 4.8 ± 0.9 |

^{a}Groups of 5 hamsters were intranasally administered 10^{6.0} TCID₅₀ of virus per animal in a 0.1 mL inoculum. After 44 days, the animals were challenged with 10^{6.0} TCID₅₀ of rwt, and lungs and nasal turbinates were harvested four days later.
^{b}The quantity of virus present in each tissue sample was determined on LLC-MK2 cells at 32° C.
^{c}Serum hemagglutination inhibition antibody titer against PIV3 on day 42; i.e., two days before challenge. S.E., Standard error. The HAI reciprocal mean log2 titer in preinfection serum was <2.

The rcp45-456 Mutant is More Attenuated than rcp45 in Chimpanzees

Because the rcp45-456 mutant appeared over-attenuated in hamsters, we sought to determine its level of replication and immunogenicity in chimpanzees, the non-human primate that is the most closely-related to humans. Groups of chimpanzees in two separate confirmatory experiments were inoculated IN and IT with 10^{6.0} TCID₅₀ per site of either rcp45-456 or rcp45. Tracheal lavage and nasopharyngeal swab samples were collected over a period of 10 and 13 days post infection, respectively, and the virus titer in each specimen was determined. rcp45-456 and rcp45 were highly restricted in replication in the upper and lower respiratory tracts in comparison to PIV3 wt (Table 8) indicating that each mutant virus is attenuated for replication in chimpanzees. As described above (Table 6), the effect of adding the F456L mutation to cp45 was to reduce replication more than 2500-fold in the upper respiratory tract of hamsters. In contrast, when evaluated in chimpanzees the effect was a reduction of only five-fold in both the upper and lower respiratory tracts (Table 8 and FIG. 2). This difference was observed in two independent experiments, one in which two animals received rcp45 and four received rcp45-456, and a second in which each virus was administered to two animals.

TABLE 8
rcp45-456 is more attenuated for replication in the upper and lower respiratory tract
of chimpanzees than rcp45.

| | | | Virus Replication | | | | | |
|---|---|---|---|---|---|---|---|---|
| | | | Nasopharyngeal | | | | | |
| | | Dose^{a} | swab fluid | | Tracheal lavage fluid | | | Mean Peak |
| Virus used to | No. of | (log10 | No. with | | No. with | | Mean days | rhinorrhea |
| infect animal | animals | TCID₅₀/ml | virus | Titer^{b} | virus | Titer^{b} | of shedding | score |
| rcp45-456 | 6 | 6.0 | 6 | 3.4 ± 0.1^{c} | 3 | 1.2 ± 0.4 | 0.7 ± 0.3 | 0.2 ± 0.1^{d} |
| rcp45 | 4 | 6.0 | 4 | 4.1 ± 0.3^{c} | 3 | 1.9 ± 0.5 | 1.8 ± 0.6 | 1.0 ± 0.3^{d} |
| PIV3^{e} | 4 | 6.0 | 4 | 6.3 ± 0.5 | 4 | 5.2 ± 0.8 | 3.8 ± 0.5 | 3.3 ± 0.5 |

^{a}The indicated amount of virus was administered in the nose and trachea in a 1 ml inoculum at each site.
^{b}Mean peak virus titer (log₁₀TCID₅₀/ml). Virus titer was determined by TCID₅₀ on LLC-MK2 cells at 32° C.
^{c}Statistically significant difference between indicated values; p < 0.025 (Student's t-test).
^{d}Statistically significant difference between indicated values; p < 0.01 (Student's t-test).
^{e}Wt JS strain of PIV3 (data from Hall et al., 1992).

Comparison of the pattern of daily virus shedding in the upper respiratory tract showed that the rcp45-456 virus reached lower peak titers relative to rcp45, but that its shedding diminished more slowly (FIG. 2). This pattern was previously shown to be characteristic of an increased level of attenuation for RSV mutants in non-human primates (Prince et al., Infect. Immun. 26(3):1009-13, 1979). This increased level of attenuation was also reflected in a significantly lower mean peak rhinorrhea score (Table 8). Thus, the introduction of the F456L mutation into rcp45 resulted in an incremental increase in attenuation for the respiratory tract of chimpanzees.

The ts Phenotype of rcp45 and rcp45-456 is Maintained after Replication in Chimpanzees

The level of temperature sensitivity of isolates obtained from chimpanzees infected with r cp45-456 and rcp45 was examined to determine whether the two recombinants retained their ts phenotype following replication in a highly permissive host. Isolates from the two studies were evaluated separately because of the large number of isolates obtained. Analysis of isolates from two individual animals from experiment 1 is shown in Table 9, and a summary of all the animals is shown in Table 10. The level of temperature-sensitivity of identical control virus suspensions differed in the two experiments by 1° C. (Table 10), indicative of experimental variability in the assay. For example, the temperature at which plaques were not observed for rcp45-456 versus rcp45 was 38° C. and 39° C., respectively, in experiment 1 compared to 39° C. and 40° C., respectively, in experiment 2 (Table 10). This level of experimental variability is sometimes observed. Importantly, in all experiments the difference in the shut-off temperature of plaque formation between rcp45-456 and rcp45 was consistent at 1° C. Because of the experimental variability between the two studies, they are summarized separately in Table 10. Each isolate retained the ts phenotype, and the level of temperature sensitivity of the isolates did not differ from that of the control input viruses throughout the course of replication in respiratory tract of the chimpanzee. Significantly, the percentage of rcp45-456 isolates plaquing at 38° C. and 39° C. was lower than that of the rcp45 isolates indicating that the greater level of temperature sensitivity of rcp45-456 observed in vitro is maintained following replication in vivo.

TABLE 9
Both rcp45 and rcp45-456 maintain their ts phenotype following replication
in seronegative chimpanzees: analysis of samples from two individual
animals from experiment 1 and comparison of controls from two experiments

| Virus isolates or | Isolates^{c} | | Virus titer^{d} (log₁₀pfu/ml) at the indicated temperature | | | | | |
|---|---|---|---|---|---|---|---|---|
| control viruses | tissue | day | 32° C. | 36° C. | 37° C. | 38° C. | 39° C. | 40° C. |
| rcp45-456 | NP | 1 | 7.1 | <3.7 | <2.7 | <0.7 | <0.7 | <0.7 |
| (Animal #1614) | NP | 2 | 7.3 | <3.7 | <2.7 | <0.7 | <0.7 | <0.7 |
| | NP | 3 | 7.7 | <3.7 | <2.7 | <0.7 | <0.7 | <0.7 |
| | NP | 4 | 8.0 | 4.4 | 4.2 | <0.7 | <0.7 | <0.7 |
| | NP | 5 | 7.8 | 4.3 | 4.2 | <0.7 | <0.7 | <0.7 |
| | NP | 6 | 7.3 | 4.2 | 4.0 | <0.7 | <0.7 | <0.7 |
| | NP | 7 | 6.9 | 3.7 | 3.0 | <0.7 | <0.7 | <0.7 |
| | NP | 8 | 7.7 | <3.7 | 3.0 | <0.7 | <0.7 | <0.7 |
| | NP | 9 | 7.5 | 5.3 | 4.8 | <0.7 | <0.7 | <0.7 |
| | NP | 10 | 7.8 | 5.4 | 4.2 | <0.7 | <0.7 | <0.7 |
| | TL | 2 | 7.8 | 4.7 | 2.7 | <0.7 | <0.7 | <0.7 |
| | TL | 4 | 7.4 | 4.0 | <2.7 | <0.7 | <0.7 | <0.7 |
| rcp45 | NP | 1 | 7.0 | ND | 4.7 | 4.2 | <0.7 | <0.7 |
| (Animal #1616) | NP | 2 | 7.5 | ND | 4.4 | 3.7 | <0.7 | <0.7 |
| | NP | 3 | 7.3 | ND | 3.7 | <0.7 | <0.7 | <0.7 |
| | NP | 4 | 7.4 | ND | 4.5 | <0.7 | <0.7 | <0.7 |
| | NP | 5 | 6.3 | ND | 4.0 | <0.7 | <0.7 | <0.7 |
| | NP | 6 | 7.2 | ND | 4.9 | 3.4 | <0.7 | <0.7 |
| | NP | 7 | 5.8 | ND | <3.7 | <0.7 | <0.7 | <0.7 |
| | NP | 8 | 5.9 | ND | 4.5 | 2.2 | <0.7 | <0.7 |
| | NP | 13 | 6.9 | ND | 4.3 | 3.4 | <0.7 | <0.7 |
| | TL | 2 | 7.5 | 5.3 | 4.3 | 3.3 | <0.7 | <0.7 |
| | TL | 4 | 7.2 | 5.7 | 4.5 | <0.7 | <0.7 | <0.7 |
| rcp45-456^{a} | | | 8.4 | 6.0 | 4.6 | <0.7 | <0.7 | <0.7 |
| rcp45^{a} | | | 8.0 | 7.0 | 5.8 | 3.4 | <0.7 | <0.7 |
| rwta^{a} | | | 7.2 | 7.2 | 7.3 | 7.0 | 6.9 | 7.0 |
| rcp45-456^{b} | | | 8.3 | 7.0 | 5.0 | 3.0 | <0.7 | <0.7 |
| rcp45^{b} | | | 7.8 | 7.5 | 6.3 | 4.8 | 2.3 | <0.7 |
| rwt^{b} | | | 7.8 | 7.6 | 7.4 | 7.5 | 7.4 | 7.2 |

^{a}Control virus suspensions, study #1.
^{b}Control virus suspensions, study #2 (see Table 10).
^{c}virus isolates were harvested following passage of chimpanzee nasopharyngeal swabs and tracheal lavage specimens in LLC-MK2 cells at 30° C.
^{d}Virus isolates were titered on LLC-MK2 monolayer cultures at the indicated temperature for 6 days, and plaques were enumerated by immunoperoxidase staining using anti-HPIV3 HN monoclonal antibodies.
ND = not determined.

TABLE 10
Both rcp45 and rcp45-456 maintain their temperature-sensitive
phenotypes following replication in seronegative chimpanzees:
summary of two experiments

| Virus used | | | | | | |
|---|---|---|---|---|---|---|
| to infect | Total | Percentage of isolates with viral plaques | | | | |
| animal (no. | no. of | detected at indicated temperature | | | | |
| of animals) | isolates | 36° C. | 37° C. | 38° C. | 39° C. | 40° C. |
| Experiment 1 | | | | | | |
| rcp 45 (2) | 20^{a} | 100 | 95 | 70 | 0 | 0 |
| rcp45-456 (4) | 42^{b} | 83 | 60 | 0 | 0 | 0 |
| Experiment 2 | | | | | | |
| rcp45 (2) | 25^{c} | 100 | 100 | 100 | 72 | 0 |
| rcp45-456 (2) | 22^{d} | 100 | 86 | 91 | 5 | 0 |

Isolates were obtained as described in Material and Methods and th shut-off temperature of plaque formation was determined by efficiency of plaquing at the indicated temperatures (see Materials and Methods, and Table 9).
^{a}Total number includes 18 nasopharyngeal and 2 tracheal lavage isolates.
^{b}Total number includes 39 nasopharyngeal and 3 tracheal lavage isolates.
^{c}Total number includes 20 nasopharyngeal and 5 tracheal lavage isolates.
^{d}Total number includes 21 nasopharyngeal and 1 tracheal lavage isolates.

The foregoing results demonstrate that the mutation in RSV cpts530, namely substitution of the parental phenylalanine at position 521 in the L protein, confers a similar level of temperature sensitivity and attenuation when introduced into the corresponding position (456) in the L protein of PIV3. RSV and PIV3 represent separate subfamilies within the paramyxovirus family, namely Pneumovirinae and Paramyxovirinae, respectively. The two viruses share unambiguous, statistically significant sequence relatedness for their L proteins (Stec et al. Virology 183:273-287 (1991)), particularly in a region of approximately 540-570 amino acids which lies near the amino-terminus (aa 422-938 of RSV and 357-896 of PIV3) and which includes four highly-conserved polymerase motifs described by Poch et al. (J. Gen. Virol. 5:1153-62 (1990); and Stec et al. Virology 183:273-287 (1991)). The mutation described here, at position 521 in RSV and 456 in PIV3, lies within this general region but is approximately 175 residues upstream of the first conserved motif identified by Poch et al. Notably, this residue is strictly conserved, as is a leucine residue located 12 residues C-terminal to its position (FIG. 1, panel A), among thirteen heterologous paramyxoviruses tested in the present study. Despite this strict conservation, without prior identification of the 521 L mutation in the RSV cpts530 mutant and successful transfer of this mutation to a conserved position in the HPIV3 L protein, it would not have been possible to predict that this residue, out of the 2233 positions in the amino acid sequence of the PIV3 L protein, would be an appropriate target site for mutagenesis to yield a conditional-lethal attenuated recombinant. In view of these results, it is now possible to extend the methods of the invention to embrace the large number of attenuating mutations recently identified among diverse members of the Mononegavirales, particularly the paramyxoviruses (Bukreyev et al., J. Virol, 71(12), 8973-8982 (1997); Garcin et al., Virology 238(2):424-431 (1997); Juhasz et al., Vaccine 17:1416-1424 (1999); Juhasz et al., J. Virol. 71(8):5814-5819 (1997); Kato et al., EMBO J. 16(3):578-587 (1997a); Kato et al., J. Virol. 71(10):7266-7272 (1997b); Kondo et al., J. Biol Chem. 268(29):21924-21930 (1993); Kurotani et al., Genes Cells 3(2):111-124 (1998); Skiadopoulos et al., J. Virol. 72(3):1762-8 (1998); Whitehead et al., J. Virol. 73:871-877 and 73:3438-3442 (1999); Whitehead et al., Virology 247(2):232-239 (1998a); Whitehead et al., J. Virol. 73(2):871-877 (1999b); Whitehead et al., J. Virol, 72(5):4467-4471 (1998b), each incorporated herein by reference).

The present findings provide novel PIV vaccine candidates and also enable transfer of attenuating mutations from RSV to other paramyxoviruses wherein the mutation maps to a conserved parental residue/position. Further in this context, the 521 L mutation of RSV is amenable to importation into recently developed chimeric PIV3 recombinants having the hemagglutinin-neuraminidase (HN) and F genes of JS wt PIV3 replaced by those of PIV1 (Tao et al., J. Virol. 72(4), 2955-2961 (1998), incorporated herein by reference). This chimeric recombinant can be attenuated further by incorporation of one or more additional mutations identified in the cp45 mutant, which mutations have now been incorporated in their entirety (with the exception of the three mutations occurring in the F and HN genes) within an infectious, attenuated, chimeric PIV3-1 clone bearing the PIV1 HN and F genes substituted within a JS wt PIV3 background.

### Example III

### Heterologous Transfer of an Attenuating Mutation in the C Protein of Sendai Virus into a Recombinant HPIV3 Vaccine Candidate

The present example describes heterologous transfer of a known attenuating mutation in the C protein of Sendai virus (SeV), marked by a substitution of phenylalanine (F) to serine (S) at position 170 (Itoh et al., J. Gen. Virol. 78:3207-3215 (1997), incorporated herein by reference), to a corresponding position in a recombinant HPIV3 clone. As described above and illustrated in Table 4, The F170 parental sequence element maps to an identically conserved sequence position/residue F164 in the HPIV3 C protein, which element is also conserved in SeV and BPIV-3.

The F170S mutation of SeV was transferred to a recombinant HPIV3 virus rF164S by introduction of a point mutation into the C ORF of HPIV3, changing amino acid position 164 from phenylalanine (F) to serine (S). The resulting rF164S recombinant was surprisingly, more attenuated in the upper than the lower respiratory tract. This pattern is the converse of that seen with temperature-sensitive attenuating mutations, whereby inclusion of this novel mutation in recombinant, live-attenuated vaccine viruses will prove useful in reducing residual virulence in the upper respiratory tract. The rF164S recombinant also conferred protection against challenge with wildtype HPIV3.

The P and C proteins of HPIV3 are translated from separate, overlapping ORFs in the mRNA (FIG. 3). Whereas all paramyxoviruses encode a P protein, only members of the genera Respirovirus and Morbillivirus encode a C protein. Individual viruses vary in the number of proteins expressed from the C ORF and in its importance in replication of the virus in vitro and in vivo. Sendai virus (SeV) expresses four independently initiated proteins from the C ORF: C′, C, Y1, and Y2, whose translational start sites appear in that order in the mRNA (Curran, et al., Enzyme 44:244-9 (1990); Lamb et al., p. 181-214, in D. Kingsbury (ed.), The Paramyxoviruses, Plenum Press, New York (1991), whereas HPIV3 and measles virus (MeV) express only a single C protein (Bellini et al., J Virol. 53:908-19, (1985); Sanchez et al., Virology 147:177-86 (1985); Spriggs et al., J. Gen. Virol. 67:2705-2719 (1986)).

A viable recombinant SeV in which all four C-derived proteins were ablated was found to replicate extremely inefficiently in vitro (Kurotani et al., Genes Cells 3:111-124 (1998)), whereas ablation of individual C proteins had complex effects (Cadd et al., J Virol. 70:5067-74 (1996); Curran, et al., Virology 189:647-56 (1992); Latorre et al., J Virol. 72:5984-93 (1998)).

A recombinant SeV bearing a single point mutation resulting in a phenylalanine (F) to serine (S) substitution at amino acid position 170 of the C protein was attenuated in mice, but its replication in cell culture was not impaired (Garcin et al., Virology 238:424-431 (1997); Itoh et al., J. Gen. Virol. 78:3207-15 (1997)). In marked contrast to SeV, a C-minus measles virus (MeV) replicated efficiently in Vero cells (Radecke et al., Virology 217:418-21 (1996)), although it exhibited restriction of replication in human peripheral blood cells and appeared to be only somewhat attenuated in vivo (Escoffier et al., J Virol. 73:1695-8 (1999); Valsamakis et al., J Virol. 72:7754-61 (1998)).

The altered replication of the various MeV and SeV C mutants in animals suggests that this protein is a potential target for the introduction of attenuating mutations useful in the development live attenuated HPIV3 vaccines. In the present example, reverse genetics methodology was used to introduce a mutation into the C ORF of HPIV3 which created a F→S change at amino acid position 164, which corresponds to the F→S change at amino acid position 170 in a heterologous, attenuated SeV mutant.

### Cells and Viruses

HEp-2 and simian LLC-MK2 monolayer cell cultures were maintained in OptiMEM 1 (Life Technologies, Gaithersburg, Md.) supplemented with 2% fetal bovine serum, gentamicin sulfate (50 ug/mL), and 4 mM glutamine. The modified vaccinia strain Ankara (MVA) recombinant virus that expresses bacteriophage T7 RNA polymerase was generously provided by Drs. L. Wyatt and B. Moss (Wyatt et al., Virology 210:202-205 (1995)). The JS wildtype (wt) strain of PIV3 and its attenuated ts derivative, JS cp45, were propagated in LLC-MK2 cells as described previously (Hall et al., Virus Res. 22:173-184 (1992)).

cDNAs

The full-length cDNA clone encoding the complete 15462 nt antigenome {p3/7(131)²G} of the JS wt virus was described previously (Genebank accession #Z11575) (Durbin et al., Virology 235:323-332 (1997)). This clone was used as the template for the construction of a mutated cDNA encoding a phenylalanine to serine change at amino acid position 164 of the C ORF (Table 11, FIG. 3). The PmlI to BamHI fragment of p3/7(131)2G (nt 1215-3903 of the PIV3 antigenome) was subcloned into the plasmid pUC119 {pUC119(PmlI-BamHI)} which had been modified to include a PmlI site in the multiple cloning region. Site-directed mutagenesis was then performed on pUC119(PmlI-BamHI) using Kunkel's method (Kunkel et al., Methods Enzymol. 154:367-382, 1987) to introduce mutations in the C ORF.

TABLE 11

Nucleotide change introduced into rPIV3 to yield rF164S virus.
Nucleotide sequence of wt/mutated sequence¹

| | | |
|---|---|---|
| rPIV3 | Amino acid | SEQ. ID |
| designation | substitution | NO. |

| | | | |
|---|---|---|---|
| rF164S | Phe-164 to Ser | 2276-GCA AAT GTT CCA AGC GAG ATA TC | 5 |
| | | GCA AAT GTC CCA AGC GAG ATA TC | 6 |

¹The nucleotide sequence of the mutated region is shown and compared with wildtype (wt) sequence. The first nucleotide in the sequence is numbered according to its position in the complete antigenome RNA. Sequences as blocked is the P open reading frame. Nucleotides underlined are the codon for the C ORF at amino acid position 164 for HP103.

Construction of Antigenomic cDNA Encoding Virus with the F164S Mutation in the C ORF (rF164S)

A phenylalanine (F) to serine (S) change at amino acid position 164 of the C ORF was created using a mutagenic primer which introduced an A to G change at nt position 2284 of the full-length antigenome. This mutation was also silent in the P ORF. The PmlI to BamHI fragment of the full-length clone was sequenced in its entirety to confirm the presence of the introduced mutation and to confirm that other mutations had not been introduced incidently. The fragment was then separately cloned back into the full-length clone p3/7(131)2G as previously described (Durbin et al., Virology 235:323-332 (1997)) to create the antigenomic cDNA clone.

Recovery of a Recombinant C F164S Mutant from cDNA

The full-length antigenomic cDNA bearing the C F164S mutation was transfected into HEp-2 cells on six-well plates (Costar, Cambridge, Mass.) together with the support plasmids {pTM(N), pTM (P no C), and pTM (L)} using LipofectACE (Life Technologies) and infected with MVA-T7 as previously described (Durbin et al., Virology 235:323-332, 1997). pTM(P no C) is identical to the pTM(P) plasmid described previously (Durbin et al., Virology 234:74-83 (1997)) with the exception that the C transcriptional start site had been mutated from ATG to ACG, changing the first amino acid in the C ORF from methionine to threonine. After incubation at 32° C. for three days, the transfection harvest was passaged onto a fresh LLC-MK2 cell monolayer in a T25 flask and incubated for 5 days at 32° C. (referred to as passage 1). The amount of virus present in the F164S harvest was determined by plaque titration on LLC-MK2 monolayer cultures with plaques visualized by immunoperoxidase staining with PIV3 HN-specific monoclonal antibodies as described previously (Durbin et al., Virology 235:323-332 (1997)).

The F164S recombinant virus present in the supernatant of passage 1 harvest was then plaque purified three times on LLC-MK2 cells as previously described (Hall et al., Virus Res. 22:173-184 (1992)). The biologically cloned recombinant virus from the third round of plaque purification was then amplified twice in LLC-MK2 cells at 32° C. to produce virus for further characterization,

### Sequence Analysis of Recovered Recombinant Viruses

Virus was concentrated from clarified medium from infected cell monolayers by polyethylene glycol precipitation as described previously (Durbin et al., Virology 235:323-332 (1997)). The RNA was purified with TRIzol reagent (Life Technologies) following the manufacturer's recommended procedure. RT-PCR was performed with the Advantage RT-for-PCR kit (Clontech, Palo Alto, Calif.) following the recommended protocol. Control reactions were identical except that reverse transcriptase was omitted from the reaction to confirm that the PCR products were derived solely from viral RNA and not from possible contaminating cDNA plasmids. Primers were used to generate the PCR fragment spanning nucleotides 1595-3104 of the full-length antigenome. This fragment includes the entire C, D and V ORFs of the recombinant viruses. The resultant PCR products were then sequenced using cycle dideoxynucleotide sequence analysis (New England Biolabs, Beverly, Mass.).

### Protein Analysis by Immunoprecipitation

Two PIV3 C-specific antisera were raised in rabbits by the multiple antigen peptide (MAP) technique against two different C peptides (Research Genetics, Huntsville, Ala.). The two peptides spanned amino acid regions 30-44 and 60-74 of the C protein. Eight copies of each C peptide were placed on a branched carrier core and injected separately into rabbits (two rabbits per peptide) with Freund's adjuvant. The rabbits were boosted with the designated MAP at 2 and 4 weeks and bled at 4, 8, and 10 weeks. Each antiserum recognized the C peptide used as an immunogen in high titer and precipitated C protein from HPIV3 infected cells in a radioimmunoprecipitation assay (RIPA).

T25 cell monolayers of LLC-MK2 cells were infected at a multiplicity of infection (MOI) of 5 with either rF164S, recombinant JS wt virus (rJS) or were mock infected and incubated at 32° C. At 24 hours post-infection, the monolayer was washed with methionine-minus DMEM (Life Technologies) and incubated in the presence of 10 uCi/ul of ³⁵S methionine in methionine-minus DMEM for an additional 6 hours. The cells were then harvested, washed 3 times, and resuspended in 1 ml RIPA buffer {1% (w/v) sodium deoxycholate, 1% (v/v) Triton X-100, 0.2% (w/v) SDS, 150 mM NaCl, 50 mM Tris-HCl, pH 7.4}, freeze-thawed and pelleted at 6500×G. The cell extract was transferred to a fresh eppendorf tube and a mixture of both C antisera (5 ul each) was added to each sample and incubated with constant mixing for 2 hours at 4° C. 10 ul of a mixture of mAb 454/11 and 101/1, which recognize the HN glycoprotein of HPIV3 (van Wyke Coelingh et al., J Virol. 61:1473-1477 (1987)), were added to each sample to confirm that recovered virus was indeed HPIV3. Immune complexes were precipitated by adding 200 ul of a 10% suspension of protein A Sepharose beads (Sigma, St. Louis, Mo.) to each sample followed by constant mixing at 4° C. overnight. Each sample was denatured, reduced, and analyzed on a 4-12% polyacrylamide gel (NuPAGE, Novex, San Diego, Calif.) per the manufacturer's recommendations. The gel was dried and analyzed by autoradiography.

Multicycle Replication of rPIV3s

Monolayers of LLC-MK2 cells in T25 flasks were infected in duplicate with rF164S or r JS at an MOI of 0.1 and incubated at 32° C. in 5% CO₂. 250 ul samples were removed from each flask at 24 hour intervals for 5 consecutive days and were flash frozen. An equivalent volume of fresh media was replaced at each time point. Each sample was titered on LLC-MK2 cell monolayers in 96-well plates incubated for 7 days at 32° C. Virus was detected by hemadsorption and reported as log₁₀ TCID₅₀/ml.

### Animal Studies

4-6 week-old golden Syrian hamsters in groups of 21 were inoculated intranasally with 0.1 ml per animal of EMEM (Life Technologies) containing 10⁵ PFU of rF164S, rJS, cp45 (the biologically-derived live attenuated derivative of JS wt virus), or respiratory syncytial virus (RSV). On days 3, 4, and 5 post-inoculation 5 hamsters from each group, except those which received RSV, were sacrificed and the lungs and nasal turbinates harvested. The nasal turbinates and lungs were homogenized to prepare a 10% or 20% w/v suspension in L-15 (Quality Biologicals, Gaithersburg, Md.) respectively, and the samples were rapidly frozen. Virus present in the samples was titered on 96 well plates of LLC-MK2 cell monolayers incubated at 32° C. for 7 days. Virus was detected by hemadsorption and the mean log₁₀ TCID₅₀/g was calculated for each day for each group of five hamsters. Sera were collected from the remaining 6 hamsters in each group on days 0 and 28 post-inoculation. Serum antibody responses to each virus was evaluated by hemagglutination-inhibition (HAI) assay as previously described (van Wyke Coelingh et al., Virology 143:569-582 (1985)). On day 28 the remaining hamsters in each group, including those immunized with RSV, were challenged intranasally with 10⁶ PFU of biologically-derived PIV3 JS wt virus. The animals were sacrificed on day 4 post-challenge and the lungs and nasal turbinates were harvested and processed as described above. The quantity of virus present in the challenge samples was determined as described above.

African Green monkeys (AGMs) in groups of 4 animals each were inoculated intranasally and intratracheally with 10⁶ PFU of either rF164S, JSwt, or cp45 as previously described for earlier studies in rhesus monkeys (Durbin et al., Vaccine 16:1324-30 (1998)). Nasopharyngeal swab samples were collected daily for 12 consecutive days post-inoculation and tracheal lavage samples were collected on days 2, 4, 6, 8, and 10 post-inoculation. The specimens were flash frozen and stored at −70° C. until all specimens had been collected. Virus present in the samples was titered on LLC-MK2 cell monolayers in 96 well plates that were incubated at 32° C. for 7 days. Virus was detected by hemadsorption and the mean log₁₀ TCID₅₀/ml was calculated for each day. Serum was collected from each monkey on days 0 and 28, and the PIV3 HAI antibody response to experimental infection with the various mutants and the PIV3 wild type (JS) was determined. On day 28 post-inoculation, the AGMs were challenged with 10⁶ PFU of the biologically-derived PIV3 wild type virus administered in a 1 ml inoculum intranasally and intratracheally. Nasopharyngeal swab samples were collected on days 0, 1, 2, 4, 6, 8, 10, and 12 post-challenge and tracheal lavage samples were collected on days 2, 4, 6, 8, and 10 post-challenge. Specimens were flash frozen, stored, and virus present was titered as described above.

Results

Recovery of Recombinant Mutant rF164S

An HPIV3 antigenomic cDNA was prepared to encode the mutant virus rF164S, in which the C protein aa residue 164 was changed from F to S. The mutation was translationally silent in the overlapping P ORF (Table 11).

The antigenomic cDNA was transfected into HEp-2 cells along with the three PIV3 support plasmids {pTM(P no C), pTM(N), pTM(L)} and the cells were simultaneously infected with MVA expressing the T7 RNA polymerase. The efficiency of recovery of rPIV3 containing the mutation in the C ORF was compared with that of a similarly transfected-infected HEp-2 cell culture using the p3/7(131)2G, the plasmid expressing full-length PIV3 antigenome from which recombinant JSwt PIV3 (rJS) was previously recovered (Durbin et al., Virology 235:323-332 (1997)). After incubation for 3 days at 32° C. the transfected cells were harvested, and supernatant was passaged onto a fresh monolayer of LLC-MK2 cells in a T25 flask and incubated for 5 days at 32° C. (passage 1). After 5 days at 32° C., the LLC-MK2 cell monolayer of rJS and rF164S exhibited 3-4+ cytopathic effect (CPE). After three rounds of biological cloning by plaque isolation, the recombinant mutant was amplified twice in LLC-MK2 cells to produce a suspension of virus for further characterization.

To confirm that the recovered virus was indeed the expected rF164S mutant, the cloned virus was analyzed by RT-PCR using a primer pair which amplified a fragment of DNA spanning nt 1595-3104 of the HPIV3 antigenome, which includes the portion of the P gene containing the C, D, and V ORFs. The generation of PCR product was dependent upon the inclusion of RT, indicating derivation from RNA and not from contaminating cDNA. Nucleotide sequencing was conducted on the RT-PCR product to confirm the presence of the introduced mutation. The introduced mutation was confirmed to be present in the RT-PCR fragment spanning nt 1595-3104 amplified from the cloned recombinant, and other incidental mutations were not found.

Radioimmunoprecipitation assay (RIPA) was performed to compare the rF164S mutant virus with the rJS wt virus. Cells were infected, incubated in the presence of ³⁵S methionine from 24 to 30 h post-infection, and cell lysates were prepared. Equivalent amounts of the total protein were incubated with anti-C and anti-HN antibodies and the antibody was then bound to protein A sepharose beads. rJS and rF164S each encoded both HN and C proteins (FIG. 4). The C protein expressed by rF164S appeared to be of identical size as that expressed by the parent rJS and was expressed in similar quantity as well (FIG. 4).

Replication of rF164S in Cell Culture

Duplicate cultures of LLC-MK2 cell monolayers were infected with rF164S or rJS at an MOI of 0.1 and incubated for 5 days at 32° C. Medium from each culture was sampled at 24 hour intervals and this material was subsequently titered to evaluate the replication of each virus in cell culture. Replication of rF164S was essentially indistinguishable from that of the parent rJS wt with regard to both the rate of virus production and the final titer (FIG. 5) as well as the ability to replicate at elevated temperature.

### Replication of r F164S in Hamsters

The rF164S mutant virus was next compared with the rJS wt parent for the ability to replicate in the upper and lower respiratory tract of hamsters. Groups of hamsters were inoculated intranasally with 10⁵ pfu per animal of rF164S or cp45, the biologically-derived vaccine candidate. Compared to rJS, replication of rF164S was reduced one hundred to five hundred-fold in upper respiratory tract and was reduced more than ten-fold in the lower respiratory tract of the hamsters (Table 12). The hamsters which were infected with rF164S and rJS had a significant antibody response to HPIV3 and exhibited a high level of restriction of replication of PIV3 challenge virus (Table 13).

TABLE 12
The rF164s virus is attenuated in the upper and lower respiratory tracts of hamsters.

| | Mean virus titer (log₁₀TCID₅₀/g ± S.E.² on day post-infection | | | | | | Peak titer virus | |
|---|---|---|---|---|---|---|---|---|
| | Day3 | | Day 4 | | Day 5 | | (log₁₀TCID₅₀/g ± S.E.) | |
| Virus¹ | Nasal turb. | Lungs | Nasal turb. | Lungs | Nasal turb. | Lungs | Nasal turb. | Lungs |
| cp45 | 4.7 ± 0.2 | 2.7 ± 0.2 | 5.0 ± 0.5 | 2.8 ± 0.5 | 5.4 ± 0.3 | 1.6 ± 0.2 | 5.4 ± 0.3(C)³ | 2.8 ± 0.5(C)³ |
| rF164S | 4.0 ± 0.2 | 5.3 ± 0.3 | 4.7 ± 0.3 | 5.4 ± 0.2 | 4.4 ± 0.6 | 4.3 ± 0.1 | 4.7 ± 0.3(B) | 5.4 ± 0.2(B) |
| rJS | 6.7 ± 0.3 | 6.8 ± 0.5 | 7.4 ± 0.2 | 6.6 ± 0.4 | 6.6 ± 0.2 | 7.2 ± 0.2 | 7.4 ± 0.2(A) | 7.2 ± 0.2(A) |

¹Groups of 5 hamsters were inoculated intranasally with 10⁵ pfu of indicated virus. cp45 is a biologically-derived virus, and the others are recombinant viruses.
²Standard error.
³Means in each column with a different letter are significantly different (a = 0.05) by Duncan's Multiple Range test whereas those with the same letter are not significantly different

TABLE 13
The rF164S virus is immunogenic and protective against challenge
with PIV3 wt virus in hamsters.

| | | Response to Challenge | |
|---|---|---|---|
| | | Mean PIV3 titer² | |
| Immunizing | Serum HAI Antibody Titer | (log₁₀TCID₅₀/g) ± S.E. | |
| Virus¹ | (reciprocal mean log₂ ± S.E.) | Nasal turb. | Lungs |
| rF 164S | 10.8 ± 0.2 | <1.5 ± 0.0 | <1.2 ± 0.0 |
| cp45 | 11.8 ± 0.3 | <1.5 ± 0.0 | <1.2 ± 0.0 |
| rJS | 12.1 ± 0.2 | <1.5 ± 0.0 | <1.2 ± 0.0 |
| RSV | <2.0 ± 0.0 | 7.0 ± 0.2 | 4.7 ± 0.0 |

¹Indicates virus used to immunize groups of six hamsters on Day 0.
²On day 28, all hamsters were bled to determine the serum HAI antibody titer and were challenged with 10⁶ PFU biologically-derived JS wt HPIV3. Lungs and nasal turbinates were harvested on day 4 post-challenge.

Replication of rF164S in Primates

To further evaluate the attenuation phenotype and protective efficacy of rF164S, this recombinant mutant was administered to a group of four AGMs, and its replication in the upper and lower respiratory tracts was compared with that of cp45 and JSwt. Replication of rF164S was 100-fold or more reduced in the upper respiratory tract of the AGMs. The attenuation observed for this virus in the upper respiratory tract was comparable to that of cp45 (Table 14). rF164S was moderately (<10-fold) restricted in the lower respiratory tract of the AGMs. The recombinant virus induced an HAI antibody response to HPIV3 (Table 14). The immunized AGMs were challenged on day 28 with 106 PFU biologically-derived JS wt virus given IN and IT. The animals which had received rF164S or the cp45 vaccine candidate virus were completely protected against replication of challenge virus in both the upper and lower respiratory tracts of the AGMs.

TABLE 14
rF164S is attenuated and induces a serum HAI antibody response in African
Green monkeys

| | | | | Serum HAI | Mean peak titer | |
|---|---|---|---|---|---|---|
| | Mean peak titer | | | antibody titer on | (log₁₀TCID₅₀/ml) ± S.E. of | |
| | (log₁₀TCID₅₀/ml) ± S.E.² | | | day 28 | challenge virus⁴ | |
| | Nasopharyngeal | Tracheal | Number | (reciprocal mean | Nasopharyngeal | Tracheal |
| Virus¹ | swab | lavage | of animals | log₂ ± S.E.)³ | swab | lavage |
| rF164S | 4.3 ± 0.3 (C)⁵ | 5.8 ± 0.2 (B)⁵ | 4 | 12.5 ± 0.3 | <0.5 ± 0.0 | <0.5 ± 0.0 |
| cp45 | 5.1 ± 0.4 (B) | 5.3 ± 0.1 (C) | 4 | 12.0 ± 0.6 | <0.5 ± 0.0 | <0.5 ± 0.0 |
| JSwt | 6.3 ± 0.2 (A) | 6.6 ± 0.1 (A) | 6 | 13.2 ± 0.2 | | |

¹AGMs were inoculated intranasally and intratracheally with 10⁶ PFU of indicated virus on day 0.
²Standard error.
³The mean serum HAI antibody titer (reciprocal mean log2) on day 0 was ±2.0.
⁴Animals were challenged on day 28 with 106 PFU HPIV3 JS wt virus given intranasally and intratracheally.
⁵Means in each column with a different letter are significantly different α = 0.05) by Duncan's Multiple Range test whereas those with the same letter are not significantly different.
⁶Two animals in this group received the recombinant JS wt virus rJS and 4 animals received the biologically-derived JSwt virus. Peak mean titers of the two viruses was not different (6.4 vs 6.3 in the upper respiratory tract and 6.6 vs 6.7 in the lower respiratory tract). Because the peak titers were not different, the groups were combined for further analyses.

Summarizing the above example, the F170S mutation in SeV was generated biologically by adapting the virus to growth in LLC-MK2 simian cells (Garcin et al., Virology 238:424-431 (1997), and Itoh et al., J. Gen. Virol. 78:3207-15 (1997)). Although recombinant SeV with the F170S mutation demonstrates enhanced replication in vitro, this mutant is highly restricted in replication in mice, being as impaired in replication as the SeV C′/C mutant (Garcin et al., Virology 238:424-431 (1997) and Latorre et al., J Virol. 72:5984-93 (1998)). Since the C proteins of SeV and HPIV3 are only 38% homologous, it was surprising to find that importation of the F170S mutation of SeV into HPIV3 caused attenuation in vivo.

The HPIV3 recombinant rF164S, which bears the mutation at the amino acid residue of HPIV3 corresponding to position 170 of SeV, was not restricted in replication in vitro but was significantly attenuated in both the upper and lower respiratory tracts of hamsters and in the upper and lower respiratory tracts of AGMs. rF164S induced a serum antibody response comparable to that of wt PIV3 infection and completely protected both hamsters and AGMs against challenge with wt HPIV3. These results indicate that the F164S mutation will be useful in developing a live attenuated vaccine against HPIV3.

Deposit of Biological Material

The following materials have been deposited with the American Type Culture Collection, 10801 University Boulevard, Manassas, Va. 20110-2209, under the terms of the Budapest Treaty.

| Virus | Accession No. | Deposit Date |
|---|---|---|
| p3/7(131)2G | (ATCC 97989) | Apr. 18, 1997 |
| p3/7(131) | (ATCC 97990) | Apr. 18, 1997 |
| p218(131) | (ATCC 97991) | Apr. 18, 1997 |
| cpts RSV 248 | (ATCC VR 2450) | Mar. 22, 1994 |
| cpts RSV 530/1009 | (ATCC VR 2451) | Mar. 22, 1994 |
| RSV cpts530 | (ATCC VR 2452) | Mar. 22, 1994 |
| cpts RSV 248/955 | (ATCC VR 2453) | Mar. 22, 1994 |
| cpts RSV 248/404 | (ATCC VR 2454) | Mar. 22, 1994 |
| cpts RSV 530/1030 | (ATCC VR 2455) | Mar. 22, 1994 |
| RSV B-1 cp52/2B5 | (ATCC VR 2542) | Sep. 26, 1996 |
| A2 ts530-s cllcp, or | (ATCC VR 2545) | Oct. 17, 1996 |
| ts530-sites | | |
| RSV B-1 cp-23 | (ATCC VR 2579) | Jul. 15, 1997 |

Although the foregoing invention has been described in some detail by way of illustration and example for purposes of clarity of understanding, it will be obvious that certain changes and modifications may be practiced within the scope of the appended claims.

1-126. (canceled)

127. An infectious, attenuated, recombinant parainfluenza virus (PIV) comprising:
a recombinant PIV genome or antigenome and a nucleocapsid protein (N), nucleocapsid phosphoprotein (P) and a large polymerase protein (L), said recombinant PIV genome or antigenome encoding at least one amino acid substitution that confers attenuation on said recombinant virus,
wherein the location of said at least one amino acid substitution is in an amino acid sequence element that is conserved in PIV at a position of an attenuating amino acid substitution in a genome or antigenome of a negative stranded RNA virus belonging to a genus different from Respirovirus.

128. An isolated polynucleotide comprising a polynucleotide encoding the genome or antigenome of a recombinant PIV particle of claim 127.

129. An expression vector comprising:
i) a promoter operable in a mammalian cell or in vitro, operatively linked to ii) a polynucleotide according to claim 128, in turn operatively linked to a transcriptional terminator operable in a mammalian cell or in vitro.

130. A method for producing an infectious, attenuated human parainfluenza virus particle comprising:
co-expressing in a cell or in a cell-free system an expression vector according to claim 129 and human parainfluenza virus (HPIV) N, P and L proteins, wherein said HPIV N, P and L proteins are optionally expressed from additional expression vectors, thereby obtaining a an infectious, attenuated human parainfluenza virus particle comprising a recombinant PIV genome or antigenome and a nucleocapsid protein (N), nucleocapsid phosphoprotein (P) and a large polymerase protein (L), said recombinant PIV genome or antigenome encoding at least one amino acid substitution that confers attenuation on said recombinant virus,
wherein the location of said at least one amino acid substitution is in an amino acid sequence element that is conserved in PIV at a position of an attenuating amino acid substitution in a genome or antigenome of a negative stranded RNA virus belonging to a genus different from Respirovirus.

131. An infectious, attenuated human parainfluenza virus type 1 (HPIV1), human parainfluenza virus type 2 (HPIV2) or human parainfluenza virus type 3 (HPIV3) particle comprising a major nucleocapsid (N) protein, a nucleocapsid phosphoprotein (P), a large polymerase protein (L), and a partial or complete HPIV1, HPIV 2 or HPIV 3 genome or antigenome, respectively, encoding at least said N, P and L proteins, wherein said HPIV1, HPIV2 or HPIV3 genome or antigenome includes a mutation of the codon encoding phenylalanine at position 456 (F456), position 459 (F459) or at position 456 (F456) in the L protein, respectively, to a codon encoding another amino acid.

132. The HPIV1, HPIV2 or HPIV3 particle of claim 131, in which the genome or antigenome further includes a mutation of one or more of the following codons:
i) the codon encoding Y942, Y945 or Y942 of the L protein, respectively;
ii) the codon encoding L992, L995 or L992 of the L protein, respectively;
iii) the codon encoding T1558, T1661 or T1558 of the L protein, respectively;
iv) the codon encoding Q766, Q769 or Q766 of the L protein, respectively;
v) the codon encoding M1104, M1107 or M1104 of the L protein, respectively;
vi) the codon encoding Y1256, Y1259 or Y1256 of the L protein, respectively;
vii) the codon encoding 196 of the C protein of HPIV3 or the codon encoding the corresponding amino acid in HPIV1 or HPIV2;
viii) the codon encoding 1417, 1423 or 1420 of the F protein; and
ix) the codon encoding A447, A453 or A450 of the F protein.

133. The HPIV1, HPIV2 or HPIV3 particle of claim 132, in which the mutation
i) is to H;
ii) is to F;
iii) is to I;
iv) is to L;
v) is to V;
vi) is to N;
vii) is to T;
viii) is to V;
ix) is to T.

134. An infectious, attenuated human parainfluenza virus type 3 (HPIV3) particle comprising a major nucleocapsid (N) protein, a nucleocapsid phosphoprotein (P), a large polymerase protein (L), and a partial or complete HPIV 3 genome or antigenome, encoding at least said N, P and L proteins, wherein said HPIV3 genome or antigenome includes a mutation of the codon encoding phenylalanine at position 164 (F164) in the C protein to a codon encoding another amino acid.

135. The HPIV3 particle of claim 134, in which the genome or antigenome further includes a mutation of one or more of the following codons:
i) the codon encoding Y942 of the L protein, respectively;
ii) the codon encoding L992 of the L protein, respectively;
iii) the codon encoding T1558 of the L protein, respectively;
iv) the codon encoding Q766 of the L protein, respectively;
v) the codon encoding M104 of the L protein, respectively;
vi) the codon encoding Y1256 of the L protein, respectively;
vii) the codon encoding 196 of the C protein;
viii) the codon encoding 1420 of the F protein; and
ix) the codon encoding A450 of the F protein.

136. The HPIV3 particle of claim 135, in which the mutation
i) is to H;
ii) is to F;
iii) is to I;
iv) is to L;
v) is to V;
vi) is to N;
vii) is to T;
viii) is to V;
ix) is to T.

137. An isolated polynucleotide comprising a polynucleotide encoding the genome or antigenome of a HPIV1, HPIV2 or HPIV3 particle of any one of claims 131-133.

138. An isolated polynucleotide comprising a polynucleotide encoding the genome or antigenome of a HPIV3 particle of any one of claims 134-136.

139. An expression vector comprising:
i) a promoter operable in a mammalian cell or in vitro, operatively linked to ii) a polynucleotide according to claim 137, in turn operatively linked to a transcriptional terminator operable in a mammalian cell or in vitro.

140. An expression vector comprising:
i) a promoter operable in a mammalian cell or in vitro, operatively linked to ii) a polynucleotide according to claim 138, in turn operatively linked to a transcriptional terminator operable in a mammalian cell or in vitro.

141. A method for producing an infectious, attenuated human parainfluenza virus particle comprising:
co-expressing in a cell or in a cell-free system an expression vector according to claim 139 and human parainfluenza virus (HPIV) N, P and L proteins, wherein said HPIV N, P and L proteins are optionally expressed from additional expression vectors, thereby obtaining an HPIV1, HPIV2 or HPIV3 particle comprising a N protein, a P protein, a L protein, and a partial or complete HPIV1, HPIV 2 or HPIV 3 genome or antigenome, respectively, including at least a mutation of the codon encoding phenylalanine at position 456 (F456), position 459 (F495) or at position 456 (F456) in the L protein, respectively, to a codon encoding another amino acid.

142. A method for producing an infectious, attenuated human parainfluenza virus particle comprising:
co-expressing in a cell or in a cell-free system an expression vector according to claim 140 and human parainfluenza virus (HPIV) N, P and L proteins, wherein said HPIV N, P and L proteins are optionally expressed from additional expression vectors, thereby obtaining an HPIV3 particle comprising a N protein, a P protein, a L protein, and a partial or complete HPIV3 genome or antigenome including at least a mutation of the codon encoding phenylalanine at position 164 (F164) in the C protein to a codon encoding another amino acid.

143. The method of claim 141, in which at least one of said N, P and L proteins are expressed from at least one additional expression vector.

144. The method of claim 142, in which at least one of said N, P and L proteins are expressed from at least one additional expression vector.

145. An infectious, attenuated human parainfluenza virus type 1 (HPIV1) or human parainfluenza virus type 2 (HPIV2) particle comprising a major nucleocapsid (N) protein, a nucleocapsid phosphoprotein (P), a large polymerase protein (L), and a partial or complete HPIV1 or HPIV 2 genome or antigenome, respectively, encoding at least said N, P and L proteins, wherein said HPIV1 or HPIV2 genome or antigenome includes at least one mutation selected from the group consisting of:
i) the codon encoding Y942 or Y945 of the L protein, respectively;
ii) the codon encoding L992 or L995 of the L protein, respectively;
iii) the codon encoding T1558 or T1661 of the L protein, respectively;
iv) the codon encoding Q766 or Q769 of the L protein, respectively;
v) the codon encoding M104 or M1107 of the L protein, respectively;
vi) the codon encoding Y1256 or Y1259 of the L protein, respectively;
vii) the codon encoding the amino acid in HPIV1 or HPIV2 corresponding the codon encoding 196 of the C protein of HPIV3:
viii) the codon encoding 1417 or 1423 of the F protein, respectively; and
ix) the codon encoding A447 or A453 of the F protein, respectively; to a codon encoding another amino acid.

146. The HPIV1 or HPIV2 particle of claim 145, in which the mutation
i) is to H;
ii) is to F;
iii) is to I;
iv) is to L;
v) is to V;
vi) is to N;
vii) is to T;
viii) is to V;
ix) is to T.

147. An isolated polynucleotide comprising a polynucleotide encoding the genome or antigenome of a HPIV3 particle of any one of claims 145 or 146.

148. An expression vector comprising:
i) a promoter operable in a mammalian cell or in vitro, operatively linked to ii) a polynucleotide according to claim 147, in turn operatively linked to a transcriptional terminator operable in a mammalian cell or in vitro.

149. A method for producing an infectious, attenuated human parainfluenza virus particle comprising:
co-expressing in a cell or in a cell-free system an expression vector according to claim 148 and human parainfluenza virus (HPIV) N, P and L proteins, wherein said HPIV N, P and L proteins are optionally expressed from additional expression vectors, thereby obtaining an HPIV1 or HPIV2 particle comprising a N protein, a P protein, a L protein, and a partial or complete HPIV1 or HPIV 2 genome or antigenome, respectively, including at least one mutation selected from the group consisting of:
i) the codon encoding Y942 or Y945 of the L protein, respectively;
ii) the codon encoding L992 or L995 of the L protein, respectively;
iii) the codon encoding T1558 or T1661 of the L protein, respectively;
iv) the codon encoding Q766 or Q769 of the L protein, respectively;
v) the codon encoding M1104 or M1107 of the L protein, respectively;
vi) the codon encoding Y1256 or Y1259 of the L protein, respectively;
vii) the codon encoding the amino acid in HPIV1 or HPIV2 corresponding the codon encoding 196 of the C protein of HPIV3:
viii) the codon encoding 1417 or 1423 of the F protein, respectively; and
ix) the codon encoding A447 or A453 of the F protein, respectively; to a codon encoding another amino acid.


SEQUENCE LISTING (53 sequences)

SEQ ID NO: 1 - protein, 53 aa, Human parainfluenza virus 3
QGVKIITHKECSTIGINGMLFNTNKEGTLAFYTPNDITLNNSVALDPINISIE

SEQ ID NO: 2 - protein, 53 aa, Bovine parainfluenza virus 3
QGIKIITHKECQVIGINGMLFNTNREGTLATYTFDDIILNNSVALNPIDISME

SEQ ID NO: 3 - protein, 53 aa, Human parainfluenza virus 1
RGVTFLTYTNCGLIGINGIELYANKRGRDTTRGNQIIKVGPAVSIRPVDISLN

SEQ ID NO: 4 - protein, 53 aa, Human parainfluenza virus 2
QGISIIDIKRCSEMMLDTFSFRITSTFNATYVTDFSMINANIVHLSPLDLSNQ

SEQ ID NO: 5 - DNA, 23 bp, Human parainfluenza virus 3
gcaaatgttccaagcgagatatc

SEQ ID NO: 6 - DNA, 23 bp, Artificial Sequence
  Nucleotide sequence encoding F164S mutation
gcaaatgtcccaagcgagatatc

SEQ ID NO: 7 - protein, 53 aa, respiratory syncytial virus
NGCDYVSNKGVDTVSVGNTLYYVNKQEGKSLYVKGEPIINFYDPLVFPSDQFD

SEQ ID NO: 8 - protein, 53 aa, Measles virus
KILTYIAADHCPVVEVNGVTIQVGSRRYPDAVYLHRIDLGPPISLERLDVGTN

SEQ ID NO: 9 - protein, 41 aa, Human parainfluenza virus 3
NPNRMQYASLIPASVGGFNYMAMSRCFVRNIGDPSVAALAD

SEQ ID NO: 10 - protein, 41 aa, Bovine parainfluenza virus 3
NIHWMQYASLIPASVGGFNYMAMSRCFVRNIGDPTVAALAD

SEQ ID NO: 11 - protein, 41 aa, Sendai virus
GLNWLRCAVLIPANVGGFNYMSTSRCFVRNIGDPAVAALAD

SEQ ID NO: 12 - protein, 41 aa, Human parainfluenza virus 2
HPRLISRIVLLPSQLGGLNYLACSRLFNRNIGDPLGTAVAD

SEQ ID NO: 13 - protein, 41 aa, Measles virus
NNDLLIRMALLPAPIGGMNYLNMSRLFVRNIGDPVTSSIAD

SEQ ID NO: 14 - protein, 44 aa, respiratory syncytial virus
LDNIDTALTLYMNLPMLFGGGDPNLLYRSFYRRTPDFLTQAIVH

SEQ ID NO: 15 - protein, 46 aa, Human parainfluenza virus 3
LDRSVLYRIMNQEPGESSFLDWASDPYSCNLPQSQNITTMIKNITA

SEQ ID NO: 16 - protein, 46 aa, Bovine parainfluenza virus 3
LDRGVLYRIMNQEPGESSFLDWASDPYSCNLPQSQNITTMIKNITA

SEQ ID NO: 17 - protein, 46 aa, Sendai virus
LDKQVLYRVMNQEPGDSSFLDWASDPYSCNLPHSQSITTIIKNITA

SEQ ID NO: 18 - protein, 46 aa, Human parainfluenza virus 2
LGSWILYNLLARKPGKGSWATLAADPYSLNQEYLYPPTTILKRHTQ

SEQ ID NO: 19 - protein, 46 aa, Measles virus
MPEETLHQVMTQQPGDSSFLDWASDPYSANLVCVQSITRLLKNITA

SEQ ID NO: 20 - protein, 46 aa, respiratory syncytial virus
LNKFLTCIITFDKNPNAEFVTLMRDPQALGSERQAKITSGINRLAV

SEQ ID NO: 21 - protein, 47 aa, Human parainfluenza virus 3
HPKVFKRFWDCGVLNPIYGPNTASQDQIKLALSICEYSLDLFMREWL

SEQ ID NO: 22 - protein, 47 aa, Bovine parainfluenza virus 3
HPKVFKRFWDCGVLDPIYGPNTASQDQVKLALSICEYSLDLFMREWL

SEQ ID NO: 23 - protein, 47 aa, Sendai virus
HPKIFKRFWNAGVVEPVYGPNLSNQDKILLALSVCEYSVDLFMHDWQ

SEQ ID NO: 24 - protein, 47 aa, Human parainfluenza virus 2
HPKLLRRAMNLDIITPIHAPYLASLDYVKLSIDAIQWGVKQVLADLS

SEQ ID NO: 25 - protein, 47 aa, Measles virus
HPKIYKKFWHCGIIEPIHGPSLDAQNLHTTVCNMVYTCYMTYLDLLL

SEQ ID NO: 26 - protein, 47 aa, respiratory syncytial virus
EQKVIKYILSQDASLHRVEGCHSFKLWFLKRLNVAEFTVCPWVVNID

SEQ ID NO: 27 - protein, 41 aa, Human parainfluenza virus 3
NAYGSNSAISYENAVDYYQSFIGIKFNKFIEPQLDEDLTIY

SEQ ID NO: 28 - protein, 41 aa, respiratory syncytial virus
YYKLNTYPSLLELTERDLIVLSGLRFYREFRLPKKVDKEMI

SEQ ID NO: 29 - protein, 41 aa, Measles virus
NAQASGEGLTHEQCVDNWKSFAGVKFGCFMPLSLDSDLTMY

SEQ ID NO: 30 - protein, 41 aa, Sendai virus
NAQGSNTAISYECAVDNYTSFIGFKFRKFIEPQLDEDLTIY

SEQ ID NO: 31 - protein, 41 aa, Human parainfluenza virus 2
EFQHDNAEISYEYTLKHWKEISLIEFRKCFDFDPGEELSIF

SEQ ID NO: 32 - protein, 41 aa, canine distemper virus
NAHASGEGITYSQCIENWKSFAGIRFKCFMPLSLDSDLTMY

SEQ ID NO: 33 - protein, 41 aa, Simian virus 41
ELHHDNSEISYEYTLRHWKELSLIEFKKCFDFDPGEELSIF

SEQ ID NO: 34 - protein, 41 aa, Phocine distemper virus
NACVSGEGITYSQCVENWKSFAGIKFRCFMPLSLDSDLTMY

SEQ ID NO: 35 - protein, 41 aa, Hendra virus
RLKNSGESLTVDDCVKNWESFCGIQFDCFMELKLDSDLSMY

SEQ ID NO: 36 - protein, 41 aa, Simian virus 5
ELMNDNTEISYEFTLKHWKEVSLIKFKKCFDADAGEELSIF

SEQ ID NO: 37 - protein, 41 aa, Rinderpest virus
NAQASGEGLTYEQCVDNWKSFAGIRFGCFMPLSLDSDLTMY

SEQ ID NO: 38 - protein, 41 aa, Avian pneumovirus
YMNAKTYPSNLELCVEDFLELAGISFCQEFYVPSQTSLEMV

SEQ ID NO: 39 - protein, 41 aa, Newcastle disease virus
QLHADSAEISHDIMLREYKSLSALEFEPCIEYDPVTNLSMF

SEQ ID NO: 40 - protein, 56 aa, Human parainfluenza virus 3
MKLERWIRTLLRGKCDNLQMFQARYQEVMTYLQQNKVETVIMEEAWNLSVHLIQDQ

SEQ ID NO: 41 - protein, 58 aa, Bovine parainfluenza virus 3
MKLGRWIRTLLRGKCDNLKMFQSRYQGVMPFLQQNKMETVMMEEAWNLSVHLIQDIPA

SEQ ID NO: 42 - protein, 55 aa, Sendai virus
MKTERWLRTLIRGEKTKLKDFQKRYEEVHPYLMKEKVEQIIMEEAWSLAAHIVQE

SEQ ID NO: 43 - protein, 55 aa, Human parainfluenza virus 1
MKTERWLRTLIRGKKTKLRDFQKRYEEVHPYLMMERVEQIIMEEAWKLAAHIVQE

SEQ ID NO: 44 - protein, 41 aa, respiratory syncytial virus
YYKLNTYPSLLELTERDLIVLSGLRFYREFRLPKKVDLEMI

SEQ ID NO: 45 - protein, 41 aa, Human parainfluenza virus 3; identical to SEQ ID NO: 27

SEQ ID NO: 46 - protein, 41 aa, Measles virus; identical to SEQ ID NO: 29

SEQ ID NO: 47 - protein, 41 aa, Sendai virus; identical to SEQ ID NO: 30

SEQ ID NO: 48 - protein, 18 aa, Artificial Sequence
  Consensus sequence
NASEVDSFGKFFLDDLTY

SEQ ID NO: 49 - DNA, 12 bp, Human parainfluenza virus 3
ttcaataaattc

SEQ ID NO: 50 - DNA, 12 bp, Artificial Sequence
  Nucleotide sequence encoding F456L mutation
ctgaataaattc

SEQ ID NO: 51 - DNA, 11 bp, Human parainfluenza virus 3
aaaaaaggggg

SEQ ID NO: 52 - DNA, 20 bp, Human parainfluenza virus 3
gttgatggaaagcgatgcta

SEQ ID NO: 53 - DNA, 13 bp, Artificial Sequence
  Modified editing site of P mRNA
aaaaaaggggggg